(19) United States
(12) Patent Application Publication
(10) Pub. No.: US 2022/0354957 A1
(43) Pub. Date: Nov. 10, 2022

(54) PEPTIDES TARGETING MACROPHAGES, AND CONJUGATES, COMPOSITIONS, AND USES THEREOF

(71) Applicant: Twinpig Biolab, Inc., Seoul (KR)

(72) Inventors: Hyunsu BAE, Seoul (KR); Ik-Hwan HAN, Namyangju-si, Gyeonggi-do (KR); Moonkyu KANG, Namyangju-si, Gyeonggi-do (KR); Hongseo CHOI, Seoul (KR); Jeongyoon CHOI, Seoul (KR); Heekyung LEE, Yongin-si, Gyeonggi-do (KR)

(21) Appl. No.: 17/665,231

(22) Filed: Feb. 4, 2022

Related U.S. Application Data
(60) Provisional application No. 63/185,503, filed on May 7, 2021.

(51) Int. Cl. A61K 47/64; C07K 14/435; A61K 47/65; A61P 35/00; A61P 37/02

(52) U.S. Cl. CPC A61K 47/6415; C07K 14/43572; A61K 47/65; A61P 35/00; A61P 37/02; A61K 38/00

(57) ABSTRACT

The present disclosure relates to polypeptides that target macrophages, and conjugates, compositions, and uses thereof. The polypeptides are selective for M2-type, M1-type, and/or M0-type macrophages, such as tumor-associated macrophages.

### CROSS-REFERENCE TO RELATED APPLICATIONS

This application claims benefit of the filing date of U.S. Appl. No. 63/185,503, filed May 7, 2021, the disclosure of which is incorporated by reference herein in their entirety.

### REFERENCE TO SEQUENCE LISTING SUBMITTED ELECTRONICALLY

The content of the electronically submitted sequence listing in ASCII text file (Name: 3409-0001US01_Sequence_Listing_ST25.txt; Size: 20 KB; and Date of Creation: Feb. 4, 2022) filed with the application is incorporated herein by reference in its entirety.

### FIELD OF THE INVENTION

The present disclosure relates to polypeptides that target macrophages, and conjugates, compositions, and uses thereof. The polypeptides are selective for M2-type, M1-type, and/or M0-type macrophages.

### BACKGROUND OF THE INVENTION

Macrophages are important innate immune cells found inalmost all tissues and originate from the bone marrow and circulate in the blood and are differentiated in tissues via extravasation. These macrophages are classified into three phenotypes: M0 macrophages, tumor-suppressing M1 macrophages, and tumor-supporting M2 macrophages.

M0 macrophages are inactivated macrophages differentiated from human peripheral monocytes.

M1 macrophages have a strong ability to present antigens, and are generally activated by interferon-gamma, lipopolysaccharide (LPS), and tumor necrosis factor (TNF)-alpha, and have pro-inflammatory and bactericidal effects.

M2 macrophages are known to promote immunosuppression, tumorigenesis and angiogenesis by releasing various extracellular matrix components, angiogenesis and chemotactic factors. Generally, the M2 macrophages are induced by IL-4 and IL-13 and are distinguished from M1 macrophages in which the M2 macrophages express unique M2 markers such as arginase-1, mannose (MMR, CD206), andscavenger receptors (SR-A, CD204).

Melittin is a major component of bee venom of honeybee (Apis mellifera L.) and is an amphiphilic peptide with 26 amino acid residues. The melittin has membrane-perturbing effects such as pore formation, fusion and vesicle formation. The melittin has been used in tumor-bearing rat studies because of its cell toxicity against tumor cells and its ability to inhibit cell growth or induce cell death and necrosis (Russell, Cancer Immunol Immunother. 2004; 53:411-421).

In addition, conventional techniques using melittin are related to a composition for treating arteriosclerosis containing melittin (KR Appl. Pub. No. 10-2011-0117789), a composition that inhibits the activity of fibroblast-like-synovial cells containing melittin (KR Appl. Pub. No. 10-2011-0117788), and the like. Pharmaceutical compositions that selectively kill M2-type macrophages using melittin have been identified (KR Appl. Pub. No. 10-2019-0021765). Compositions containing melittin conjugated to anticancer drugs are described in KR Appl. Pub. No.10-2019-0053334.

### SUMMARY OF THE INVENTION

Disclosed herein are polypeptides comprising the amino acid sequence of X1-X2-Thr-X4-Gly-Leu-X7-Ala-Leu-Ile-X11-Trp-Ile-X14-Arg-Lys-Arg-X18-X19 (SEQ ID NO:3), wherein X1 is an amino acid other than valine, X2 is an amino acid other than leucine, X4 is an amino acid other than threonine, X7 is an amino acid other than proline, X11 is an amino acid other than serine, X14 is an amino acid other than lysine, X18 is an amino acid other than glutamine, and/or X19 is an amino acid other than glutamine. In some embodiments, the X1 is alanine (SEQ ID NO:4), the X2 is alanine (SEQ ID NO:5), the X4 is alanine (SEQ ID NO:6), the X7 is alanine (SEQ ID NO:7), the X11 is alanine (SEQ ID NO:8), the X14 is alanine (SEQ ID NO:9), the X18 is alanine (SEQ ID NO:10), the X19 is alanine (SEQ ID NO:11), or any combinations thereof.

Also disclosed herein is a polypeptide comprising the amino acid sequence of any one of SEQ ID NOS:12-35. Also disclosed herein is a polypeptide comprising the amino acid sequence of any one of SEQ ID NOS:49-55.

Also disclosed herein are conjugates comprising the polypeptides disclosed herein and a second therapeutic drug. In some embodiments, the second therapeutic drug is KLA, alpha-defensin-1, BMAP-28, brevenin-2R, buforin IIb, cecropin A-magainin 2 (CA-MA-2), cecropin A, cecropin B, chrysophsin-1, D-K6L9, gomesin, lactoferricin B, LL27, LTX-315, magainin 2, magainin IIbombesin conjugate (MG2B), pardaxin, doxorubicin, methotrexate, entinostat, cladribine, pralatrexate, lorlatinib, maytansine DM1, maytansine DM3, maytansine DM4, or combinations thereof.

The conjugates can further comprise a linker that links the polypeptides to the second therapeutic drug. In some embodiments, one or both ends of the linkers comprise a functional group selected from the group consisting of carbodiimide, N-hydroxysuccinimide ester (NHS ester), imidoester, pentafluoropheny ester, hydroxymethyl phosphine, maleimide, haloacetyl, pyridyldisulfide, thiosulfonate, vinylsulfone, EDC (1-ethyl-3 -(3 -dimethylaminopropyl) carbodiimide), DCC (N,N′ -dicyclohexylcarbodiimide), SATA (succinimidyl acetylthioacetate), sulfo-SMCC (sulfosuccinimidyl-4-(NDmaleimidomethyl) cyclohexane-1-carboxylate), DMA (dimethyl adipimidate 2HCl), DMP (dimethylpimelimidate 2HCl), DMS (dimethyl Suberimidate 2HCl), DTBP (dimethyl 3,3′-dithiobispropionimidate 2HCl), sulfo-SIAB (sulfosuccinimidyl (4-iodoacetyl)aminobenzoate), SIAB (succinimidyl(4-iodoacetyl) aminobenzoate), SBAP (succinimidyl 3-(bromoacetamido) propionate), SIA (succinimidyliodoacetate), SM(PEG)n (succinimidyl)-([Nmaleimidopropionamido]-ethyleneglycol ester, wherein n=2, 4, 6, 8, 12 or 24), SMCC(succinimidyl-4-(N-Dmaleimidomethyl)cyclohexane-1-carboxylate), LCSMCC (succinimidyl 4-(N-maleimidomethyl)cyclohexane-1-carboxy-(6-amidocaproate)), sulfo-EMCS (N-κester), EMCS (N-εsulfo-GMBS(N-γester), GMBS (N-γester), sulfo-KMUS (N-κester), sulfo-MBS (mmaleimidobenzoyl-Nhydroxysulfosuccinimide ester), MBS (m-maleimidobenzoyl-Nhydroxysuccinimide ester), sulfo-SMPB (sulfosuccinimidyl 4-(p-maleimidophenyl)butyrate), SMPB (succinimidyl 4-(pmaleimidophenyl)butyrate), AMAS (N-α-maleimido-acetoxysuccinimide ester), BMPS (N-β-maleimidopropyloxysuccinimide ester), SMPH (succinimidyl 6-[(β-maleimidopropionamido)hexanoate]), PEG12-SPDP (2-pyridyldithiol-tetraoxaoctatriacontane-N-hydroxysuccinimide), PEG4-SPDP, sulfo-LCSPDP (sulfosuccinimidyl 6- [3′-(2-pyridyldithio)propionamido]hexanoate), SPDP (succinimidyl 3-(2-pyridyldithio) propionate), LC-SPDP (succinimidyl 6-[3′-(2-pyridyldithio)propionamido]hexanoate), SMPT (4-succinimidyloxycarbonyl-alpha-methylalpha(2-pyridyldithio)toluene), DSS (disuccinimidyl suberate), BS (PEG)5 (bis(succinimidyl) penta(ethylene glycol)), BS(PEG)9 (bis(succinimidyl) nona(ethylene glycol)), BS3 (bis[sulfosuccinimidyl]suberate), BSOCOES (bis[2-(succinimidooxycarbonyloxy)ethyl]sulfone), PDPH (3-(2-pyridyldithio)propionyl hydrazide), DSG (disuccinimidyl glutarate), DSP (dithiobis[succinimidyl propionate]), BM(PEG)n (1,8-bismaleimido-ethyleneglycol, n=2 or 3), BMB (1,4-bismaleimidobutane), BMDB (1,4-bismaleimidyl-2,3 -dihydroxybutane), BMH (bismaleimidohexane), BMOE (bismaleimidoethane), DTME (dithiobismaleimidoethane), TMEA (tris(2-maleimidoethyl) amine), DSS (disuccinimidyl suberate), DST (disuccinimidyl tartarate), DTSSP (3,3′-dithiobis[sulfosuccinimidylpropionate]), EGS (ethylene glycol bis[succinimidylsuccinate]), sulfo-EGS (ethylene glycol bis[sulfosuccinimidylsuccinate]), TSAT (tris-succinimidyl aminotriacetate), DFDNB (1,5-difluoro-2,4-dinitrobenzene), and combinations thereof.

Also disclosed herein are pharmaceutical compositions comprising the polypeptides or conjugates disclosed herein and a pharmaceutically acceptable carrier. In some embodiments, the polypeptides are in a concentration of 0.05 μg/ml to 100 μg/ml. In some embodiments, the compositions are in a dosage form suitable for subcutaneous or intravenous administration. In some embodiments, the compositions are in a lyophilized or encapsulated form.

Disclosed herein are methods of decreasing M2-type macrophages or treating an M2-type macrophage-mediated disease in a subject in need thereof, comprising administering the polypeptides disclosed herein to the subject. In some embodiments, the polypeptides comprise an amino acid sequence of SEQ ID NO:3, 4, 5, 7, or 8. In some embodiments, the polypeptides decrease M2-type macrophages compared to a polypeptide having the amino acid sequence of SEQ ID NO:2. In some embodiments, the disease is a cancer. In some embodiments, the cancer is melanoma, prostate cancer, lung cancer, breast cancer, colon cancer, pancreatic cancer, or other solid tumors having M2-type tumor-associated macrophages in a cancer microenvironment. In some embodiments, the cancer is hepatocellular cancer. In some embodiments, the disease is a fibrosis-related disease, end-stage liver disease, kidney disease, idiopathic pulmonary fibrosis (IPF), heart failure, many chronic autoimmune diseases, including scleroderma, rheumatoid arthritis, Crohn' s disease, ulcerative colitis, myelofibrosis and systemic lupus erythematosus, tumor invasion and metastasis, chronic graft rejection and the pathogenesis of many progressive myopathies, liver cirrhosis and fibrosis, benign prostatic hyperplasia, or prostatitis. In some embodiments, the disease is lung fibrosis.

Also disclosed herein are methods of decreasing M1-type macrophages or treating an M1-type macrophage-mediated disease in a subject in need thereof, comprising administering the polypeptides disclosed herein to the subject. In some embodiments, the polypeptides comprise an amino acid sequence of SEQ ID NO:3, 4, 5, 7, 8, or 11. In some embodiments, the polypeptides decrease M1-type macrophages compared to a polypeptide having the amino acid sequence of SEQ ID NO:2. In some embodiments, the disease is a chronic inflammatory disease including septic shock, multiple organ dysfunction syndrome (MODS), atopic dermatitis, rheumatoid arthritis, or autoimmune disorders. In some embodiments, the disease is sepsis, which includes septic shock.

Also disclosed are methods of decreasing M0-type macrophages or treating an M0-type macrophage-mediated disease in a subject in need thereof, comprising administering the polypeptides disclosed herein to the subject. In some embodiments, the polypeptides comprise an amino acid sequence of SEQ ID NO:3, 4, 5, 6, 7, 8, 9, 10, or 11. In some embodiments, the polypeptides decrease M0-type macrophages compared to a polypeptide having the amino acid sequence of SEQ ID NO:2.

Also disclosed herein are uses of the peptides and/or conjugates disclosed herein for the treatment of M2-type macrophage-mediated diseases in subjects in need thereof. Also disclosed herein are the peptides and/or conjugates disclosed herein for use in the treatment of M2-type macrophage-mediated diseases in subjects in need thereof. Also disclosed herein are the use of the peptides and/or conjugates disclosed herein for the manufacture of medicaments for treatment of M2-type macrophage-mediated disease in subjects in need thereof.

Also disclosed herein are uses of the peptides and/or conjugates disclosed herein for the treatment of M1-type macrophage-mediated diseases in subjects in need thereof. Also disclosed herein are the peptides and/or conjugates disclosed herein for use in the treatment of M1-type macrophage-mediated diseases in subjects in need thereof. Also disclosed herein are the use of the peptides and/or conjugates disclosed herein for the manufacture of medicaments for treatment of M1-type macrophage-mediated disease in subjects in need thereof.

Also disclosed herein are uses of the peptides and/or conjugates disclosed herein for the treatment of M0-type macrophage-mediated diseases in subjects in need thereof. Also disclosed herein are the peptides and/or conjugates disclosed herein for use in the treatment of M0-type macrophage-mediated diseases in subjects in need thereof. Also disclosed herein are the use of the peptides and/or conjugates disclosed herein for the manufacture of medicaments for treatment of M0-type macrophage-mediated disease in subjects in need thereof.

### BRIEF DESCRIPTION OF THE FIGURES

FIGS. 1A-1F. Polarization of THP-1-derived macrophages. THP-1 cells were treated with PMA for M0 macrophages, and then incubated with LPS and IFN-γ for M1 macrophages and IL-4 and IL-13 for M2 macrophages. Polarization of macrophages was assessed by markers of M1, such as IL-12, CXCL10, and CD86, and M2, such as IL-10, TGF-β, arginase 1, and CD206. Macrophages treated with LPS and IFN-γ showed increased M1 markers (FIGS. 1D, 1E, and 1F) and macrophages treated with IL-4 and IL-13 showed increased M2 markers compared to M0 (FIGS. 1A, 1B, 1C, and 1F).

FIGS. 2A-2C. Affinity of TAMpep fragments in THP-1-derived M2 macrophages. To determine the major amino site of TAMpep binding to M2 macrophages, affinity test was conducted by using TAMpep and fragments of TAMpep (amino acid sequences provided in FIG. 2A) conjugated with FITC in THP-1-derived M2 macrophages (Scrambled—SEQ ID NO:48; TAMpep—SEQ ID NO:1; TAMpep114—SEQ ID NO:49; TAMpep120—SEQ ID NO:50; TAMpep820—SEQ ID NO:51; TAMpep822—SEQ ID NO:52; Mpep—SEQ ID NO:2; TAMpep1026—SEQ ID NO:53; TAMpep1226—SEQ ID NO:54; and TAMpep1526 SEQ ID NO:55). TAMpep (including 26 amino acids) showed high affinity of over 90% and Mpep (removed 7 amino acids from C terminus) showed the second highest affinity with over 45% in M2 macrophages. Fragments of TAMpep (removed over 10 amino acids from C terminus or over 4 amino acids from N terminus) showed low affinity compared with the peptide of 26 amino acids (FIGS. 2B and 2C).

FIGS. 3A-3C. Cytotoxicity of TAMpep fragments in THP-1-dervied M2 macrophages. TAMpep fragments were tested in a cytotoxicity assay in THP-1-derived M2 macrophages. TAMpep showed a high cytotoxic value of 0.815 μM at IC50 and while other peptide fragments did not show a cytotoxic effect in M2 macrophages.

FIGS. 4A-4D. Hemolysis of TAMpep and Mpep. To determine hemolysis of TAMpep and Mpep, peptides were treated with increasing concentrations (0.1-50 μM) in mouse RBC. TAMpep showed 6.669 μM at IC50 and Mpep showed >50 μM at IC50 (FIGS. 4A and 4B). In addition, TAMpep and Mpep conjugated to dKLA showed 1.122 μM and >50 μM at IC50, respectively (FIGS. 4C and 4D).

FIGS. 5A-5C. Affinity of TAMpep and Mpep in THP-1-derived macrophages. To compare whether TAMpep and Mpep adhere more specifically to M2 macrophages among subtypes of macrophages, the peptides conjugated with FITC were treated with M0, M1, and M2 macrophages polarized from THP-1 cells and analyzed by FACs. Both TAMpep and Mpep showed significantly more high affinity in M2 macrophages compared to M0 and M1 macrophages (FIGS. 5A and 5B). Additionally, TAMpep showed high affinity in M2 macrophages by immunofluorescence microscopy (FIG. 5C).

FIGS. 6A-6D. Cytotoxicity of TAMpepK and MpepK in THP-1-derived macrophages. To assess whether TAMpep and Mpep conjugated to dKLA induce selectively apoptosis, M2 macrophages were treated with increasing concentrations of TAMpepK or MpepK (0.01-10 μM). TAMpepK and MpepK induced apoptosis in M2 macrophages compared to M0 and M1 macrophages (FIGS. 6A and 6B). Furthermore, expression of caspase-3, which is related to apoptosis, was increased in M2 macrophages compared to other subtype macrophages (FIGS. 6C and 6D).

FIGS. 7A-7E. Affinity of Mpep by alanine substitution library in THP-1-derived macrophages. To find the key amino acid sequence in the adhesion ability of Mpep in M2 macrophages, the alanine-substituted library of Mpep was used. In M2 macrophages, the affinity of peptides was decreased when alanine was substituted in the third T (threonine), 6th L (leucine), ninth L (leucine), twelfth W (tryptophan), thirteenth I (isoleucine), sixteenth K (lysine) and 17th R (arginine). In addition, the affinity of the peptides was reduced in the peptides (A13-16 and A05) substituted for the sixth L (leucine) through the ninth L (leucine) and the third T (threonine), the fifteenth K (lysine), the sixteenth R (arginine), the seventeenth K (lysine), and the nineteenth Q (glutamine). The peptides (A9 and A18) substituted the second L (leucine) and eleventh S (serine) showed increased affinity in M2 macrophages (FIGS. 7A-7E). Mpep amino acid sequence in each of FIGS. 7B-7E is SEQ ID NO:2.

FIGS. 8A-8C. Cytotoxicity of TAMpepK in M2 macrophages and human melanoma cells. To determine whether TAMpepK induces more apoptosis and binding in M2 macrophages than melanoma cells, each of the TAMpep and TAMpepK was treated with THP-1-derived M2 macrophages or Sk-Mel-28 cells (FIGS. 8A and 8C). TAMpepK showed low IC50 value (1.055 μM) in M2 macrophages compared to melanoma cells (IC50: 3.583 μM) (FIG. 8B) and expression of caspase-3 was also increased in M2 macrophages compared to melanoma cells (FIG. 8C).

FIGS. 9A-9C. Proliferation and migration in melanoma cells by conditioned medium of M2 macrophages treated with TAMpepK. To test whether TAMpepK inhibit proliferation and migration of melanoma cells induced by M2 macrophages, conditioned medium of M0, M1 and M2 macrophages pretreated without or with TAMpepK (1 μM) and the conditioned medium treated in melanoma cells were prepared. Proliferation of melanoma cells was increased by conditioned medium of M2 macrophages while inhibited in conditioned medium of M2 macrophages pretreated with TAMpepK (FIG. 9A). Moreover, conditioned medium of M2 macrophage pretreated with TAMpepK inhibited migration of melanoma cells while the migration of melanoma cells were increased by conditioned medium of M2 macrophages (FIGS. 9B and 9C).

FIGS. 10A-10D. Anti-cancer effect of TAMpepK in mouse model of melanoma. To assess the anti-cancer effect of TAMpepK in vivo, murine melanoma cells (B 16F10 cell line) were injected subcutaneously in the right flank of C57BL6J mice and TAMpepK was injected intraperitoneally every 3 days after a week. Mice treated with TAMpepK showed significantly reduced tumor volume and weight compared with PBS group (FIGS. 10A, 10C, and 10D). On the other hand, the body weight of mice was not significantly changed between the PBS and TAMpepK group (FIG. 10B).

FIGS. 11A-11C. Effect of TAMpepK targeting M2-like TAMs in mouse model of melanoma. To determine whether TAMpepK reduces M2-like TAMs in a mouse model of melanoma, macrophages were isolated from tumor tissues and analyzed by FACs. M2-like TAMs (F4.80+ and CD206+ cells) were reduced significantly in TAMpepK group compare to PBS group (FIGS. 11A and 11B). However, M1-like TAMs (F4/80+ and CD86+ cells) did not show a change between PBS and TAMpepK groups (FIGS. 11A and 11B). Additionally, the change in tumor microenvironment through the M1/M2 ratio was analyzed. TAMpepK group showed increased rate of M1 macrophages by reducing M2 macrophages compared to the PBS group (FIG. 11C).

FIGS. 12A-12D. Anti-cancer effect of TAMpepK and MpepK in mouse model of melanoma. This study was done to determine the anti-cancer effect of MpepK in a melanoma model. As shown in FIG. 12, tumor volume and weight were reduced in both TAMpepK and MpepK groups (FIGS. 12A-12C), and survival rate was extended in the MpepK group compared to the PBS group (FIG. 12D).

FIGS. 13A-13E. Effect of TAMpepK and MpepK targeting M2-like TAMs in mouse model of melanoma. To determine whether MpepK induces a change in the tumor microenvironment in melanoma, M1/M2 ratio of macrophages and CD8 exhaustion were analyzed by FACS. M2-like TAMs (F4.80+ and CD206+ cells) were reduced in TAMpepK and MpepK groups compare to the PBS group. However, M1-like TAMs (F4/80+ and CD86+ cells) did not show a change in all group (FIGS. 13A and 13B). M1/M2 ratio was significantly increased in the TAMpepK and MpepK groups compared to the PBS group (FIG. 13C). In addition, exhaustion marker such as PD-1 and LAGS in CD8+ T cells was significantly reduced in the TAMpepK and MpepK groups compared to the PBS group (FIGS. 13D and 13E).

FIGS. 14A and 14B. Differentiation of THP-1-derived M2 macrophages by conditioned medium of prostate tumor cells (TCM). To determine polarization of M2 macrophages by conditioned medium of prostate cancer cells (TCM), THP-1-derived macrophages were incubated with TCM. TCM-treated macrophages showed increased mRNA expression of M2 markers, such as arginase 1, CD206 and CD163, and decreased mRNA expression of M1 markers, such as NOS2 and CCR7, compared with M0 macrophages (FIGS. 14A and 14B).

FIGS. 15A-15C. Proliferation and migration in prostate cancer cells by conditioned medium of M2 macrophages. As shown in FIGS. 15A-15C proliferation and migration of cancer cells were increased by THP-1-dervied M2 macrophages. This study tested whether M2 macrophages polarized by TCM induce proliferation and migration of prostate cancer cells. Conditioned medium of macrophages treated with TCM (M-TCM) showed increased proliferation and migration of prostate cancer cells, similar to conditioned medium of THP-1-derived M2 macrophages (FIGS. 15A-15C).

FIGS. 16A and 16B. Cell viability of macrophages by TAMpepK or MpepK. To assess whether TAMpepK and MpepK reduce cell viability of M2 macrophages differentiated by TCM, THP-1-derived macrophages were treated with TAMpepK and MpepK (1 μM) (FIG. 16A). TAMpepK and MpepK resulted in induction of apoptosis in macrophages treated with TCM, similarly to M2 macrophages (FIG. 16B).

FIGS. 17A-17C. Proliferation and migration in prostate cancer cells by conditioned medium of M2 macrophages treated with TAMpepK and MpepK. Conditioned medium of M2 macrophages and M2-like TAMs induced by TCM increased proliferation and migration of prostate cancer cells (PC3 cells) (FIGS. 17A-17C). However, conditioned medium of M2 macrophages and M2-like TAMs pretreated with TAMpepK and MpepK significantly reduced proliferation and migration of PC3 cells compared to the group of M2 macrophages or M2-like TAMs (FIGS. 17A-17C).

FIGS. 18A and 18B. Invasion in prostate cancer cells by conditioned medium of M2 macrophages treated with TAMpepK and MpepK. PC3 cells were treated with conditioned medium of macrophages. Conditioned medium of M2 macrophages and M2-like TAMs induced by TCM increased invasion of PC3 cells. However, conditioned medium of M2 macrophages and M2-like TAMs pretreated with TAMpepK and MpepK significantly reduced invasion of PC3 cells compared to the group of M2 macrophages or M2-like TAMs (FIGS. 18A and 18B).

FIGS. 19A-19F. Effect of TAMpepK and MpepK in mouse model of prostate cancer. To assess anti-cancer effect of TAMpepK and MpepK in a prostate cancer model, TRAMP-C2 cells were injected subcutaneously in the right flank of C57BL6J mice and TAMpep, dKLA, TAMpepK and MpepK were injected intraperitoneally every 3 days after a week. Mice treated with TAMpepK and MpepK showed significantly reduced tumor volume and weight compared with the PBS group (FIGS. 19B, 19C, 19E, and 19F)). On the other hand, the body weight of mice was not significantly changed between all groups (FIG. 19D).

FIGS. 20A-20D. Effect of TAMpepK and MpepK in proliferation and EMT of prostate cancer model. To determine the anti-cancer effect of TAMpepK and MpepK in tumor growth and EMT of prostate cancer model, expression of PCNA as a proliferative marker and E-cadherin, vimentin, fibronectin, TGF-β, and MMP9 as EMT (epithelial-mesenchymal transition) markers were measured in tumor tissues. Expression of PCNA was reduced in the TAMpepK and MpepK groups (FIGS. 20C and 20D). For the EMT markers, E-cadherin, known as a epithelial cell marker, was increased in TAMpepK and MpepK groups (FIGS. 20A and 20D), while vimentin and fibronectin, known as mesenchymal markers, were reduced in TAMpepK and MpepK groups (FIGS. 20B and 20D). Moreover, expression of TGF-β and MMP9 as related to EMT was also reduced in TAMpepK and MpepK groups (FIG. 20D). Thus, these findings suggest that TAMpepK and MpepK have anti-cancer effect by inhibiting tumor growth and metastasis targeting M2-like TAMs in prostate cancer.

FIGS. 21A-21E. Anti-cancer effect of TAMpepK and MpepK in a colon cancer model. To determine anti-cancer effect of TAMpepK and MpepK in tumor growth of colon cancer model, tumor tissues were measured for volume and weight. Mice treated with TAMpepK and MpepK showed significantly reduced tumor volume and weight compared to the PBS group, whereas the tumor weight was not significantly changed in MpepK (FIGS. 21A-21E).

FIGS. 22A-22C. Effect of MpepK in a mouse model of lung fibrosis. To determine whether MpepK has therapeutic effect for inhibition of lung fibrosis, mouse model of lung fibrosis was established by intratracheally administrating bleomycin. Lung fibrosis induced by bleomycin was decreased by MpepK (FIG. 22B). Additionally, gene expression related to fibrosis such as fos12, collagen type 1 and fibronectin 1 was significantly reduced in MpepK compared to PBS (FIG. 22C).

FIGS. 23A-23E. Effect of TAMpepK and MpepK in a mouse model of breast cancer. To determine the anti-cancer effect of TAMpepK and MpepK in breast cancer, the 4^{th} mammary orthotopic mouse model of breast cancer was established. TAMpepK and MpepK showed decreased tumor volume and weight compared to the PBS group (FIGS. 23B-23D). Moreover, gene expression of arginase 1 known as M2 macrophage marker was significantly reduced in MpepK compared to PBS (FIG. 23E).

FIGS. 24A-24C. Effect of TAMpepK and MpepK in lung metastasis of breast cancer. Lung metastasis was decreased in the MpepK group compared to the PBS group (FIGS. 24A-24C).

FIGS. 25A-25C. Cytotoxicity of polypeptides selective for M2-type, M1-type, and/or MO type macrophages. Polypeptides selective for M2-type, M1-type, and/or M0 type macrophages were tested in a cytotoxicity assay in THP-1-derived M2, M1 and M0 macrophages. Polarized cells were treated with MpepK, A12K, A14K, A17K, A18K, A22K, A25K or A26K peptides. MpepK showed a high cytotoxic value of 1.121 μM at IC₅₀ in M2 macrophages and while A26K showed a high cytotoxic value of 1.192 μM at IC₅₀ in M1 macrophages. Also, A17K, A22K and A25K showed similar cytotoxicity of MpepK at 1.5 μM, in M2 macrophages while A26K showed over 50% inhibition of viability at 1.5 μM, in M1 macrophages, compared to control.

FIGS. 26A-26B. Cytotoxicity and effects of A26K in in vitro sepsis model, LPS-stimulated M1 (LPS-M1) macrophages. A26K, the most selective polypeptide for M1 macrophages, was tested in in vitro sepsis model, LPS-stimulated M1 (LPS-M1) macrophages. Cell viability was analyzed using the CCK-8 assay. Expression levels of pro-inflammatory genes (IL-8, TNF-α, NF-kB, IL-1β and CXCL10) were quantified by real-time quantitative PCR. To examine the cytotoxicity of A26K in LPS-M1 macrophages, M0, M1, and LPS-M1 macrophages were treated with 1.5 μM of A26K. A26K showed significant cytotoxic effects in LPS-M1 macrophages and M1 macrophages. To further examine the expression levels of pro-inflammatory genes, M0, M1, and LPS-M1 macrophages were treated with 1.5 μM of A26K for 1h. LPS (1 m/ml) stimulation significantly increased the expression of IL8, TNF-α, IL-1β, NF-kB and CXCL10, compared to M0 macrophages. A26K treatment significantly inhibited the enhanced expression levels of IL8, TNF-α, IL-1β, NF-kB and CXCL10 by LPS stimulation.

FIGS. 27A-27B. Effects of A17K or A22K in in vitro lung fibrosis model, TGF-β1-induced A549 cells cocultured with IL-4 and IL-13 induced THP-1 macrophages. Using a cell coculture system, TGF-β1-induced A549 cells were cocultured with IL-4 and IL-13 induced THP-1 macrophages. It was clearly detected morphological alteration in A549 from oval epithelial cells to spindle shaped fibroblast-like cells. A17K or A22K intervention markedly blocked the spindle-like mesenchymal morphology phenotype of EMT in A549 cells stimulated by cocultured with IL-4 and IL-13 induced macrophages. A17K or A22K treatment significantly enhanced the expression of E-cadherin, EMT inhibition marker and reduced the expression of α-SMA, FMT enhancement marker in A549 cells compared with those of M2 macrophage alone.

FIGS. 28A-28D. Effects of MpepK in mouse model of hepatocellular carcinoma. To assess anti-cancer effect of MpepK in vivo, mouse hepa 1-6 cells were injected subcutaneously in right flank of C57BL/6J mice. 12 days after cell inoculation, MpepK was injected intraperitoneally every 3 days. As a result, there was no significant difference in body weight change between the groups. On the other hand, mice treated with MpepK of all doses (100, 200 and 400 nmol/kg) showed significantly reduced tumor volume compared with PBS group, and survival rate was significantly extended in MpepK groups (100, 200 and 400 nmol/kg) compared to PBS group.

### DETAILED DESCRIPTION OF THE INVENTION

The term “melittin” (MEL) in the present disclosure is a peptide that constitutes a main component of bee venom having a sequence such as Gly-Ile-Gly-Ala-Val-Leu-Lys-Val-Leu-Thr-Thr-Gly-Leu-Pro-Ala-Leu-Ile-Ser-Trp-Ile-Lys-Arg-Lys-Arg-Gln-Gln (SEQ ID NO:1). The term “bee venom (BV)” as used herein is a mixture of acidic and basic secretions produced in the abdomen of bees (Apismellifera) and has a colorless bitter liquid form. Main components thereof are melittin, and apamin as a peptide and mast cell degranulating (MCD) peptides, and phospholipase A2 (PLA2) as an enzyme and the like. In addition, the BV contains various trace amounts of components.

It has been determined that a peptide in which the first 7 amino acids of melittin have been removed, such as Val-Leu-Thr-Thr-Gly-Leu-Pro-Ala-Leu-Ile-Ser-Trp-Ile-Lys-Arg-Lys-Arg-Gln-Gln (SEQ ID NO:2; MEL826 or Mpep), can be mutated as SEQ ID NOS:3-11 (Mpeps, or each Mpep) to selectively target M0-type, M1-type, or M2-type macrophages.

Accordingly, disclosed herein are polypeptides comprising the amino acid sequence of X1-X2-Thr-X4-Gly-Leu-X7-Ala-Leu-Ile-X11-Trp-Ile-X14-Arg-Lys-Arg-X18-X19 (SEQ ID NO:3), wherein X1 is an amino acid other than valine, X2 is an amino acid other than leucine, X4 is an amino acid other than threonine, X7 is an amino acid other than proline, X11 is an amino acid other than serine, X14 is an amino acid other than lysine, X18 is an amino acid other than glutamine, and/or X19 is an amino acid other than glutamine. In some embodiments, the X1 is alanine (SEQ ID NO:4), the X2 is alanine (SEQ ID NO:5), the X4 is alanine (SEQ ID NO:6), the X7 is alanine (SEQ ID NO:7), the X11 is alanine (SEQ ID NO:8), the X14 is alanine (SEQ ID NO:9), the X18 is alanine (SEQ ID NO:10), the X19 is alanine (SEQ ID NO:11), or any combinations thereof (Table 1). Such polypeptides can be used alone as active ingredients or therapeutic drugs, or in combination with other active ingredients or therapeutic drugs.

TABLE 1

| SEQ | |
|---|---|
| ID NO: | Amino Acid Sequence |
| 3 | X1-X2-Thr-X4-Gly-Leu-X7-Ala-Leu-Ile-X11- |
| | Trp-Ile-X14-Arg-Lys-Arg-X18-X19 |
| 4 | Ala-X2-Thr-X4-Gly-Leu-X7-Ala-Leu-Ile-X11- |
| | Trp-Ile-X14-Arg-Lys-Arg-X18-X19 |
| 5 | X1-Ala-Thr-X4-Gly-Leu-X7-Ala-Leu-Ile-X11- |
| | Trp-Ile-X14-Arg-Lys-Arg-X18-X19 |
| 6 | X1-X2-Thr-Ala-Gly-Leu-X7-Ala-Leu-Ile-X11- |
| | Trp-Ile-X14-Arg-Lys-Arg-X18-X19 |
| 7 | X1-X2-Thr-X4-Gly-Leu-Ala-Ala-Leu-Ile-X11- |
| | Trp-Ile-X14-Arg-Lys-Arg-X18-X19 |
| 8 | X1-X2-Thr-X4-Gly-Leu-X7-Ala-Leu-Ile-Ala- |
| | Trp-Ile-X14-Arg-Lys-Arg-X18-X19 |
| 9 | X1-X2-Thr-X4-Gly-Leu-X7-Ala-Leu-Ile-X11- |
| | Trp-Ile-Ala-Arg-Lys-Arg-X18-X19 |
| 10 | X1-X2-Thr-X4-Gly-Leu-X7-Ala-Leu-Ile-X11- |
| | Trp-Ile-X14-Arg-Lys-Arg-Ala-X19 |
| 11 | X1-X2-Thr-X4-Gly-Leu-X7-Ala-Leu-Ile-X11- |
| | Trp-Ile-X14-Arg-Lys-Arg-X18-Ala |

Also disclosed herein is a polypeptide comprising the amino acid sequence of any one of SEQ ID NOS:12-35. Also disclosed herein is a polypeptide comprising the amino acid sequence of any one of SEQ ID NOS:49-55.

The terms “polypeptide,” “peptide,” and “protein,” used interchangeably herein, refer to a polymeric form of amino acids of any length conjugated via an amide bond (or peptide bond). NH₂ refers to the free amino group present at the amino terminus of a polypeptide. COOH refers to the free carboxyl group present at the carboxyl terminus of a polypeptide.

According to the present disclosure, the peptides can be obtained by various methods well known in the art. For example, the peptides can be prepared using gene recombination and protein expression systems, or by method of synthesizing the peptides in vitro via chemical synthesis such as peptide synthesis, by a cell-free protein synthesis method, and/or the like. Also disclosed herein are conjugates comprising the polypeptides disclosed herein and a second therapeutic drug. In some embodiments, the second therapeutic drug is dKLA (SEQ ID NO:47), alpha-defensin-1, BMAP-28, brevenin-2R, buforin IIb, cecropin A-magainin 2 (CA-MA-2), cecropin A, cecropin B, chrysophsin-1, D-K6L9, gomesin, lactoferricin B, LL27, LTX-315, magainin 2, magainin IIbombesin conjugate (MG2B), pardaxin, doxorubicin, methotrexate, entinostat, cladribine, pralatrexate, lorlatinib, maytansine DM1, maytansine DM3, maytansine DM4, or combinations thereof.

The term “conjugate” of the present disclosure refers to a conjugate in which an Mpep peptide and a second therapeutic drug are conjugated to each other and can target a macrophage. The conjugate can bind to, e.g., a M2-type macrophage targeted by the drug and damage the mitochondria of the macrophage to inhibit tumor growth and metastasis and can suppress the cancer by selectively suppressing angiogenesis around the tumor. That is, the conjugates of the present disclosure can have improved activity compared to second therapeutic drugs alone. However, the present disclosure is not limited thereto.

The conjugates can further comprise a linker that links the polypeptides to the second therapeutic drug. Linkers can be derived from naturally occurring multi-domain proteins or empirically designed. See, Chen, X. et al., Adv. Drug Deliv. Rev. 65:1357-1369 (2013). Linkers can include flexible linkers, rigid linkers, and in vivo cleavable linkers. In addition to the role in linking the functional domains together (as in flexible and rigid linkers) or releasing free functional domain in vivo (as in in vivo cleavable linkers), linkers can provide other advantages in the production of fusion proteins, such as improving biological activity, increasing expression yield, and achieving desirable pharmacokinetic profiles. The linkers can be small, medium, and large linkers with average lengths of 4.5±0.7, 9.1±2.4, and 21.0±7.6 residues, respectively. In some embodiments, the amino acids can be polar uncharged or charged residues, which constitute approximately 50% of the naturally encoded amino acids.

Flexible linkers are usually applied when the joined domains require a certain degree of movement or interaction. They are generally composed of small, non-polar (e.g., Gly) or polar (e.g., Ser or Thr) amino acids. The small size of these amino acids provides flexibility and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. The most commonly used flexible linkers have sequences comprising primarily of stretches of Gly and Ser residues (“GS” linker). An example of the most widely used flexible linker has the sequence of (Gly-Gly-Gly-Gly-Ser)n (SEQ ID NO:36). By adjusting the copy number “n”, the length of this GS linker can be optimized to achieve appropriate separation of the functional domains, or to maintain necessary inter-domain interactions.

Rigid linkers have been successfully applied to keep a fixed distance between the domains and to maintain their independent functions. Alpha helix-forming linkers with the sequence of (EAAAK)n (SEQ ID NO:37) have been applied to the construction of many recombinant fusion proteins. Another type of rigid linkers has a Pro-rich sequence, (XP)n, with X designating any amino acid, such as Ala, Lys, or Glu. Rigid linkers exhibit relatively stiff structures by adopting a-helical structures or by containing multiple Pro residues. In many circumstances, they separate the functional domains more efficiently than the flexible linkers. The length of the linkers can be easily adjusted by changing the copy number to achieve an optimal distance between domains. As a result, rigid linkers are chosen when the spatial separation of the domains is critical to preserve the stability or bioactivity of the fusion proteins.

In some embodiments, cleavable linkers are introduced to release free functional domains in vivo. For example, a disulfide linker (LEAGCKNFFPR↓SFTSCGSLE) (SEQ ID NO:38) based on a dithiocyclopeptide containing an intramolecular disulfide bond formed between two cysteine (Cys) residues on the linker, as well as a thrombin-sensitive sequence (PRS) between the two Cys residues can be used. The dithiocyclopeptide sequence (CRRRRRREAEAC) (SEQ ID NO:39) contains an intramolecular disulfide bond between 2 Cys residues, as well as a peptide sequence sensitive to the secretion signal processing proteases resident in the yeast secretory pathway.

The linkers can also comprise cell-penetrating peptides, which can enhance the cellular uptake of the peptides disclosed herein. Cell-penetrating linkers can comprise, e.g., 5-30 amino aicds, and can be cationic, amphipathic, or hydrophobic. Examples of cell-penetrating linkers include RLRWR (SEQ ID NO:40), GRPRESGKKRKRKRLKP (SEQ ID NO:41), GRKKRRQRRRPPQ (SEQ ID NO:42), RYIRS (SEQ ID NO:43), RRMKWKK (SEQ ID NO:44), R8-12 (SEQ ID NO:45), and RRRRRRRRRFFC (SEQ ID NO:46). See, Bohmova, E. et al., Physiol. Res. 67 (Supp. 2):5267-5279 (2018), esp. Tables 1-3, incorporated herein by reference.

For example, the conjugates can be obtained by conjugating a peptide dKLA (SEQ ID NO:47; d(KLAKLAKKLAKLAK)) to an Mpep (SEQ ID NO:3, 4, 5, 6, 7, 8, 9, 10, or 11) via a GGGGS linker (SEQ ID NO:36).

Alternatively, the conjugates can be obtained by conjugating anticancer drugs such as doxorubicin, methotrexate, entinostat, cladribine, pralatrexate, and lorlatinib to the Mpep via an SPDP linker. Alternatively, the conjugates can be obtained by conjugating maytansine DM1, maytansine DM3 and maytansine DM4 to the Mpep without a linker. However, the present disclosure is not limited thereto. That is, the conjugates of the present disclosure can be in a form in which an Mpep is directly conjugated to an anticancer drug or is conjugated thereto via a linker. However, the present disclosure is not limited thereto.

According to the present disclosure, the linker can bind to the drug and the Mpep via an amine, carboxyl or sulfhydryl group on a n Mpep and anticancer drug. However, the present disclosure is not limited thereto. See KR Appl. Pub. No. 10-2019-0053334 for compositions containing melittin conjugated to anticancer drugs.

In some embodiments, one or both ends of the linkers comprise a functional group of carbodiimide, N-hydroxysuccinimide ester (NHS ester), imidoester, pentafluoropheny ester, hydroxymethyl phosphine, maleimide, haloacetyl, pyridyldisulfide, thiosulfonate, vinylsulfone, EDC (1-ethyl-3-(3 -dimethylaminopropyl)carbodiimide), DCC (N,N′-dicyclohexylcarbodiimide), SATA (succinimidyl acetylthioacetate), sulfo-SMCC (sulfosuccinimidyl-4-(NDmaleimidomethyl) cyclohexane-1-carboxylate), DMA (dimethyl adipimidate2HCl), DMP (dimethylpimelimidate-2HCl), DMS (dimethyl Suberimidate-2HCl), DTBP (dimethyl 3,3′-dithiobispropionimidate 2HCl), sulfo-SIAB (sulfosuccinimidyl (4-iodoacetyl) aminobenzoate), SIAB (succinimidyl(4-iodoacetyl)aminobenzoate), SBAP (succinimidyl 3-(bromoacetamido)propionate), SIA (succinimidyl iodoacetate), SM(PEG)n (succinimidyl-([Nmaleimidopropionamido]-ethyleneglycol ester, wherein n=2, 4, 6, 8, 12 or 24), SMCC(succinimidyl-4-(N-Dmaleimidomethyl)cyclohexane-1-carboxylate), LCSMCC (succinimidyl 4-(N-maleimidomethyl)cyclohexane-1-carboxy-(6-amidocaproate)), sulfo-EMCS (N-nester), EMCS (N-εsulfo-GMBS(N-γester), GMBS (N-γester), sulfo-KMUS (N-κester), sulfo-MBS (mmaleimidobenzoyl-Nhydroxysulfosuccinimide ester), MBS (m-maleimidobenzoyl-Nhydroxysuccinimide ester), sulfo-SMPB (sulfosuccinimidyl 4-(p-maleimidophenyl)butyrate), SMPB (succinimidyl 4-(pmaleimidophenyl)butyrate), AMAS (N-α-maleimido-acetoxysuccinimide ester), BMPS (N-β-maleimidopropyloxysuccinimide ester), SMPH (succinimidyl 6-[(β-maleimidopropionamido)hexanoate]), PEG12-SPDP (2-pyridyldithiol-tetraoxaoctatriacontane-N-hydroxysuccinimide), PEG4-SPDP, sulfo-LCSPDP (sulfosuccinimidyl 6-[3′-(2-pyridyldithio)propionamido]hexanoate), SPDP (succinimidyl 3-(2-pyridyldithio) propionate), LC-SPDP (succinimidyl 6-[3′-(2-pyridyldithio) propionamido]hexanoate), SMPT (4-succinimidyloxycarbonyl-alpha-methylalpha(2-pyridyldithio)toluene), DSS (disuccinimidyl suberate), BS (PEG)5 (bis(succinimidyl) penta(ethylene glycol)), BS(PEG)9 (bis(succinimidyl) nona(ethylene glycol)), BS3 (bis[sulfosuccinimidyl]suberate), BSOCOES (bis[2-(succinimidooxycarbonyloxy) ethyl]sulfone), PDPH (3-(2-pyridyldithio)propionyl hydrazide), DSG (disuccinimidyl glutarate), DSP (dithiobis[succinimidyl propionate]), BM(PEG)n (1,8-bismaleimido-ethyleneglycol, n=2 or 3), BMB (1,4-bismaleimidobutane), BMDB (1,4-bismaleimidyl-2,3-dihydroxybutane), BMH (bismaleimidohexane), BMOE (bismaleimidoethane), DTME (dithiobismaleimidoethane), TMEA (tris(2-maleimidoethyl) amine), DSS (disuccinimidyl suberate), DST (disuccinimidyl tartarate), DTSSP (3,3′-dithiobis[sulfosuccinimidylpropionate]), EGS (ethylene glycol bis[succinimidylsuccinate]), sulfo-EGS (ethylene glycol bis[sulfosuccinimidylsuccinate]), TSAT (tris-succinimidyl aminotriacetate), DFDNB (1,5-difluoro-2,4-dinitrobenzene), or combinations thereof.

According to the present disclosure, the peptides can contain a targeting sequence, tag, labeled residue, and/or additional amino acid sequence designed for a specific purpose to increase the half-life or stability of the peptides. Further, the peptides of the present disclosure can be conjugated to coupling partners such as effectors, drugs, prodrugs, toxins, peptides, and/or delivery molecules. In some embodiments, the peptides of the present disclosure can be conjugated to coupling partner such as RNA, DNA or antibodies. See Shoari et al., Pharmaceutics 13:1391, pp. 1-32 (2021).

In some embodiments, to prolong the in vivo half-life, increase the stability, and/or reduce the clearance of the peptides disclosed herein, the peptides can be modified by, but are not limited to, conjugation to a carrier protein, conjugation to a ligand, conjugation to an antibody, PEGylation, polysialylation HESylation, recombinant PEG mimetics, nanoparticle attachment, nanoparticulate encapsulation, cholesterol fusion, iron fusion, acylation, amidation, glycosylation, side chain oxidation, phosphorylation, biotinylation, microsphere or microsphere polymer drug delivery system, or the addition of a surface active material, amino acid mimetics, or unnatural amino acids.

According to the present disclosure, the peptides can be prepared in the form of a pharmaceutically acceptable salt. Specifically, the salt can be formed by adding an acid thereto. For example, the salt can be formed by adding the following substances to the peptide: inorganic acids (e.g. hydrochloric acid, hydrobromic acid, phosphoric acid, nitric acid, sulfuric acid, etc.), organic carboxylic acids (e.g., acetic acid, halo acetic acid such as trifluoroacetic acid, propionic acid, maleic acid, succinic acid, malic acid, citric acid, tartaric acid, salicylic acid), acidic sugars (glucuronic acid, galacturonic acid, gluconic acid, ascorbic acid), acidic polysaccharides (e.g., hyaluronic acid, chondroitin sulfate, arginic acid), organic sulfonic acids (e.g., methanesulfonic acid, p-toluene sulfonic acid) including sulfonicacid sugar esters such as chondroitin sulfate, or the like.

Also disclosed herein are compositions, e.g., pharmaceutical compositions, comprising the polypeptides or conjugates disclosed herein and a pharmaceutically acceptable carrier.

According to the present disclosure, the peptides or conjugates can be used for humans. However, the peptides or conjugates can be administered to livestock such as cattle, horses, sheep, pigs, goats, camel, antelope, or pets such as dogs or cats, in which, e.g., an inflammatory disease or cancer occurs.

The route and mode of administration for administering the composition for preventing or treating cancer according to the present disclosure are not particularly limited. As long as the composition can reach a target site, any route and mode of administration can be used. Specifically, the composition can be administered via various routes, that is, orally or parenterally. Non-limiting examples of the route of administration can include ocular, oral, rectal, topical, intravenous, intraperitoneal, intramuscular, intraarterial, transdermal, nasal, or inhalation route. Further, the composition can be administered using any device capable of moving the activesubstance to the target cell. In some embodiments, the compositions are in dosage forms suitable for subcutaneous or intravenous administration. In some embodiments, the compositions are in lyophilized or encapsulated form.

According to the present disclosure, the pharmaceutical compositions can further comprise a pharmaceutically acceptable carrier, excipient or diluent commonly used in the preparation of the pharmaceutical composition. The carrier can include a non-naturally occurring carrier.

According to the present disclosure, the term “pharmaceutically acceptable” means to represent a characteristic that is not toxic to cells or humans exposed to the composition.

The pharmaceutical composition can be formulated in a form of oral dosage forms such as powders, granules, tablets, capsules, suspensions, emulsions, syrups, aerosols, etc., external preparations, suppositories, and sterile injectable solutions according to a conventional method. Any formulation can beused as long as it is used for the prevention or treatment of the intended disease or cancer. Thus, the present disclosure is not limited thereto.

The carriers, excipients and diluents that can be contained in the pharmaceutical composition can include, for example, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, polycaprolactone (PCL), polylactic acid (PLA), poly-L-lactic acid (PLLA), mineral oil, and//or the like.

The formulations can be prepared using diluents or excipients such as fillers,extenders, conjugation agents, wetting agents, disintegrants, and surfactants which are commonly used.

Solid preparations for oral administration include tablets, pills, powders, granules, capsules, etc. Such solid preparations can be prepared by mixing the composition with at least one excipient such as starch, calcium carbonate, sucrose or lactose, and gelatin. Further, in addition to simple excipients, lubricants such as magnesium stearate and talc can be used.

Liquid preparations for oral administration include suspensions, liquid solutions, emulsions, syrups, etc. In addition to water and liquid paraffin, which are commonly used simple diluents, various excipients, for example, wetting agents, sweeteners, fragrances, preservatives, and the like can be contained in the liquid preparation. Preparations for parenteral administration can include sterilized aqueous solutions, non-aqueous solvent, suspending agent, emulsions, lyophilized preparations, suppositories, and the like. The non-aqueous solvent and suspending agent can include propylene glycol, polyethylene glycol, vegetable oil such as olive oil, and injectable ester such as ethyl oleate. As a base for suppositories, witepsol, macrogol, tween 61, cacao butter, laurin, glycerogelatin, and the like can be used.

The compositions of the present disclosure can further include a lubricant, a wetting agent, a sweetening agent, a flavoring agent, an emulsifying agent, a suspending agent, a preservative, and the like in addition to the above ingredients. Suitable pharmaceutically acceptable carriers and formulations are described in detail in Remington's Pharmaceutical Sciences (19th ed., 1995). The composition of the present disclosure is formulated by using a pharmacologically acceptable carrier and/or excipient according to a method that can be easily performed by those skilled in the art to be prepared in a unit dose form or prepared by introduction into a multi-dose container. In this case, the formulation can also be a form of solutions, suspensions, or emulsions in oils or aqueous media or a form of excipients, powders, granules, tablets or capsules, and can additionally include a dispersant or a stabilizer. The term “administration” used herein means providing a predeterminedcomposition of the present disclosure to a subject by any suitable method.

The composition of the present disclosure can be administered parenterally, by subcutaneous infusion, or topical administration (transdermal administration) via the skin but is not limited thereto.

A suitable dose of the pharmaceutical composition can be variously prescribed by factors such as a formulation method, an administration type, age, weight, and gender of a patient, a pathological condition, food, an administration time, an administration route, an excretion rate, and response susceptibility. The oral dose of the composition of the present disclosure can be 0.1 mg/kg to 10 mg/kg (body weight) per day, 0.5 mg/kg to 1 mg/kg (body weight), or any doses or ranges derived therefrom but is not limited thereto. In addition, when the composition of the present disclosure is administered to a subject in need thereof to remove tumor-associatedmacrophages, the dose thereof can be 0.01 ug/ml to 100 ug/ml, 0.05 ug/ml to 100 ug/ml, 0.1 ug/ml to 100 ug/ml, 0.1 ug/ml to 70 ug/ml, 0.1 ug/ml to 50 ug/ml, 0.1 ug/ml to 40 ug/ml, 0.1 ug/ml to 30 ug/ml, 0.1 ug/ml to 25 ug/ml or any doses or ranges derived therefrom, but is not limited thereto.

The term “subject” used herein refers to humans and nonhumans, including all animals, such as monkeys, dogs, goats, pigs, or mice. Such subjects can be in need of treatment of diseases in which symptoms of various cancers or inflammatory diseases can be improved by administering the peptides or compositions thereof of the present disclosure.

The term “phospholipase A2 (PLA2)” used herein is an enzyme functioning to generating fatty acids by hydrolyzing glycerol at the second carbon position, which catalyzes the hydrolytic activity by specifically recognizing an sn-2 acyl bond of phospholipid to release arachidonic acid and lysophospholipid. The PLA2 is commonly found even in mammalian tissues as well as bacteria, insects, andsnake venom.

In some aspects of the present disclosure for achieving the above purpose provides a method of preparing an Mpep-anticancer drug conjugate, the method including conjugating an Mpep and an anticancer drug to each other.

Disclosed herein are methods of decreasing M2-type macrophages or treating an M2-type macrophage-mediated disease in a subject in need thereof, comprising administering polypeptides or compositions thereof as disclosed herein to the subject. In some embodiments, the polypeptides comprise an amino acid sequence of SEQ ID NO:3, 4, 5, 7, or 8. In some embodiments, the polypeptides decrease M2-type macrophages compared to a polypeptide having the amino acid sequence of SEQ ID NO:2. In some embodiments, the disease is a cancer. In some embodiments, the cancer is melanoma, prostate cancer, lung cancer, breast cancer, colon cancer, pancreatic cancer, or other solid tumors having M2-type tumor-associated macrophages in a cancer microenvironment. In some embodiments, the cancer is a hepatocellular cancer. In some embodiments, the disease is a fibrosis-related disease, end-stage liver disease, kidney disease, idiopathic pulmonary fibrosis (IPF), heart failure, many chronic autoimmune diseases, including scleroderma, rheumatoid arthritis, Crohn's disease, ulcerative colitis, myelofibrosis and systemic lupus erythematosus, tumor invasion and metastasis, chronic graft rejection and the pathogenesis of many progressive myopathies, liver cirrhosis and fibrosis, benign prostatic hyperplasia, or prostatitis. In some embodiments, the disease is lung fibrosis.

Also disclosed herein are methods of decreasing M1-type macrophages or treating an M1-type macrophage-mediated disease in a subject in need thereof, comprising administering the polypeptides or compositions thereof as disclosed herein to the subject. In some embodiments, the polypeptides comprise an amino acid sequence of SEQ ID NO:3, 4, 5, 7, 8, or 11. In some embodiments, the polypeptides decrease M1-type macrophages compared to a polypeptide having the amino acid sequence of SEQ ID NO:2. In some embodiments, the disease is a chronic inflammatory disease including septic shock, multiple organ dysfunction syndrome (MODS), atopic dermatitis, rheumatoid arthritis, or autoimmune disorders. In some embodiments, the disease is sepsis, which includes septic shock.

Also disclosed are methods of decreasing M0-type macrophages or treating an M0-type macrophage-mediated disease in a subject in need thereof, comprising administering the polypeptides or compositions thereof as disclosed herein to the subject. In some embodiments, the polypeptides comprise an amino acid sequence of SEQ ID NO:3, 4, 5, 6, 7, 8, 9, 10, or 11. In some embodiments, the polypeptides decrease M0-type macrophages compared to a polypeptide having the amino acid sequence of SEQ ID NO:2.

The Mpep polypeptides disclosed herein can selectively target M2, M1, and/or MO macrophages. As used herein, “selective” means a preference for or greater binding or affinity to one or more types of macrophages over another type, such as by but not limited to at least ¼-fold, at least ⅓-fold, at least ½-fold, at least 1-fold, at least 2-fold, at least 3-fold, at least 5-fold, etc., or any folds or ranges derived therefrom.

In some aspects of the present disclosure for achieving the above purpose provides pharmaceutical compositions for the prevention or treatment of tumor-associated macrophage-mediated diseases.

According to the present disclosure, the composition can be a pharmaceutical composition for the prevention or treatment of cancer growth and metastasis via removal of M2-type tumor-associated macrophage. However, the present disclosure is not limited thereto.

The term “prevention” according to the present disclosure refers to any actionthat inhibits or delays tumor growth and metastasis using the conjugate of the present disclosure.

The term “treatment” according to the present disclosure refers to any actionin which the symptoms of the disease, such as an inflammatory disease or cancer, tumor growth, and/or metastasis, are reduced, inhibited, or beneficially altered using the peptides disclosed herein.

According to the present disclosure, the term “anticancer drug” is a generic term for drugs used for treating cancer, such as chemotherapy drugs. The anticancer drug can be a compound or pro-apoptotic peptide. However, the present disclosure is not limited thereto.

According to the present disclosure, the term “cancer” refers to a tumor abnormally grown due to the autonomous overgrowth of body tissues, or a disease related to the tumor. In some embodiments, the cancer is melanoma, prostate cancer, lung cancer, breast cancer, colon cancer, pancreatic cancer, or other solid tumors having M2-type tumor-associated macrophages in a cancer microenvironment. According to the present disclosure, the anticancer drugs can be doxorubicin, methotrexate, entinostat, cladribine, pralatrexate, lorlatinib, maytansine DM1, maytansine DM3, and maytansine DM4. However, the present disclosure is not limited thereto.

According to the present disclosure, the term “pro-apoptosis” refers to the process in which the cell leads to death while the cell actively consumes ATP, whichis bioenergy. The typical apoptosis process proceeds via cell shrinkage, regular cleavage of DNA, and fragmentation of cell membranes. Apoptosis can be induced when cells fail to maintain their normal function due to abnormal cell division, radiation, ultraviolet radiation, bacterial infection or viral infection.

According to the present disclosure, the pro-apoptotic peptide can be dKLA, alpha-defensin-1, BMAP-28, brevenin-2R, buforin IIb, cecropin A-magainin 2 (CA-MA-2), cecropin A, cecropin B, chrysophsin-1, D-K6L9, gomesin, lactoferricin B, LLL27, LTX-315, magainin 2, magainin II-bombesin conjugate (MG2B), pardaxin, or combinations thereof. However, the present disclosure is not limited thereto.

The term “tumor-associated macrophage (TAM)” of the present disclosure refers to a macrophage that plays an important role in the overall tumor microenvironment such as cancer growth and metastasis. The tumor-associated macrophages present around the tumor are closely related to the growth and metastasis of tumor cells. Tumor-associated macrophages are classified into two phenotypes: tumor-suppressing M1 macrophage or tumor-supporting M2 macrophage. M2-type tumor-associated macrophages produce cytokines such as IL-10, TGFbeta, and CCL18, which promote cancer growth, and suppress anti-tumor activity of T cells and NK cells via surface receptors. These tumor-associated macrophages (TAM) can be differentiated from monocytes and macrophages originating from bone marrow, yolk sac or extramedullary hematopoiesis. In some embodiments, TAM can be isolated from the bone marrow. However, the present disclosure is not limited thereto.

In other aspects of the present disclosure for achieving the above purpose provides a method of preventing or treating tumor-associated macrophage mediated diseases, the method including administering the conjugate or a pharmaceuticalcomposition containing the same to a subject in need thereof.

In other aspects of the present disclosure for achieving the above purpose provides use of the Mpep-anticancer drug conjugate for prevention or treatment of the tumor-associated macrophage-mediated diseases.

The term “therapeutically effective amount” used herein refers to an amountof an Mpep effective for treating the intended disease, such as an inflammatory disease, cancer, or tumor-associated macrophage-mediated diseases.

The Mpep-anticancer drug conjugate of the present disclosure is an anticancer substance targeting the M2-type tumor-associated macrophage (TAM), and has an excellent effect of selectively selecting the M2-type tumor-associated macrophage (TAM). Thus, the conjugation method between an Mpep and the anticancer drug can be used for delivery of the drug targeting the M2-type tumor-associatedmacrophage.

The method for preventing or treating the tumor-associated macrophage mediated diseases of the present disclosure, particularly the method for preventing or treating Lewis lung cancer or inflammatory disease includes not only treating the disease itself before the development of symptoms, but also inhibiting or avoiding the symptoms thereof by administering the Mpep. In the management of a disease, a preventive or therapeutic dose of a specific active ingredient will vary depending on the nature and severity of the disease or condition, and a route by which the active ingredient is administered. The dose thereof can be 0.1 mg/kg to 10 mg/kg (body weight) per day, 0.2 mg/kg to 8 mg/kg (body weight) per day, 0.3 mg/kg to 5 mg/kg (body weight) per day, 0.4 mg/kg to 3 mg/kg (body weight) per day, 0.5 mg/kg to 1 mg/kg (body weight) per day, or any doses or ranges derived therefrom, but is not limited thereto. The oral dose of the composition of the present disclosure can be 0.1 mg/kg to 10 mg/kg (body weight) per day, 0.1 mg/kg to 10 mg/kg (body weight) per day, 0.2 mg/kg to 8 mg/kg (body weight) per day, 0.3 mg/kg to 5 mg/kg (body weight) per day, 0.4 mg/kg to 3 mg/kg (body weight) per day, 0.5 mg/kg to 1 mg/kg (body weight) per day, or any doses or ranges derived therefrom but is not limited thereto. In addition, when the composition of the present disclosure is administered to a subject in need thereof to remove tumor-associatedmacrophages, the dose thereof can be 0.01 ug/ml to 100 u g/ml, 0.05 ug/ml to 100 ug/ml, 0.1 ug/ml to 100 ug/ml, 0.2 ug/ml to 70 ug/ml, 0.3 ug/ml to 50 ug/ml, 0.4 ug/ml to 40 ug/ml, 0.5 ug/ml to 30 ug/ml, 0.6 ug/ml to 25 ug/ml, or any doses or ranges derived therefrom, but is not limited thereto.

The administration can be administered once or several times a day. However, its dose and dose frequency will vary depending on the age, weight and response of an individual patient, and a suitable dosage can be easily selected by those skilled in the art that naturally consider such factors.

### EXAMPLES

Hereinafter, exemplary embodiments are provided for better understanding of the present disclosure. However, the following exemplary embodiments are provided only for understanding the present disclosure more easily, but the content of the present disclosure is limited to the following exemplary embodiments.

### Example 1

### Materials and Methods

1-1. Peptide Synthesis.

Protected amino acids and 2-(6-chloro-1Hbenzotriazole-1-yl)-1,1,3,3 -tetramethylaminium hexafluorophosphate (HCTU) were purchased from AAPPTec (Louisville, K.Y.) and AnaSpec (Fremont, Calif.). Peptide synthesis was performed on an automated PS3 peptide synthesizer (Protein Technologies, Phoenix, Ariz.) following standard Fmoc solid phase peptide synthesis chemistry. When needed, amino acids were manually coupled by incubation in a solution of amino acids and HCTU dissolved in 0.4 M N-methylmorpholine in DMF for 3 h. The coupling reaction was checked for completion by Kaiser Test. Fmoc protecting groups were removed by two 30 min incubations in 20% (v/v) piperidine in DMF. Peptides were acetylated at the N-terminus in acetic anhydride/triethylamine/DCM (1:1:5 v/v/v) for 2 h. Peptides were cleaved in TFA (trifluoroacetic acid) / TIPS (triisopropylsilane)/EDT (1,2-ethanedithiol) / DMB (1,3-dimethoxybenzene (90:2.5:2.5:5 v/v/v/v) for 2.5 h. EDT was included in the cleavage solution only for the cysteine-containing peptides. The cleaved peptides were precipitated in cold ether twice and purified by RP-HPLC (Agilent 1200, Santa Clara, Calif.) using Phenomenex Fusion-RP C18 semi-preparative column (Torrance, Calif.) in H₂O (0.1% TFA) as a mobile phase A and ACN (0.1% TFA) as a mobile phase B. The peptides were then desalted using the HyperSep™ C18 cartridge and confirmed for purity with RP-HPLC. Molecular weights of the purified peptides were confirmed by matrix-assisted laser desorption/ionization time-of-flight mass spectrometry (MALDI-TOF MS, Bruker Daltonics, Billerica, MA).

The following peptides were synthesized according to the method described above.
TAMpep: full length melittin (SEQ ID NO:1);
Mpep: full length melittin with the first 7 amino acids removed (SEQ ID NO:2);
TAMpepK: a full-length melittin peptide (SEQ ID NO:1) attached to a linker (GGGGS)
(SEQ ID NO:36), which is attached to d-Lys-d-Leu-d-Ala-d-Lys-d-Leu-d-Ala-d-Lys-d-Lys-d-Leu-d-Ala-d-Lys-d-Leu-d-Ala-d-Lys (dKLA) (SEQ ID NO:47); and
MpepK: Mpep (SEQ ID NO:2) attached to a linker (GGGS) (SEQ ID NO:36), which is attached to d-Lys-d-Leu-d-Ala-d-Lys-d-Leu-d-Ala-d-Lys-d-Lys-d-Leu-d-Ala-d-Lys-d-Leu-d-Ala-d-Lys (dKLA) (SEQ ID NO:47).

1-2. Cells.

THP-1 cells were purchased from American Type Culture Collection (ATCC) and cultured according to their specific indications, using an RPMI 1640 medium supplemented with non-heat-treated 10% fetal bovine serum (FBS; WelGENE), 2 mM L-glutamine, 0.05 mM β-mercaptoethanol, 10 mM HEPES, 4500 mg/L glucose, 100 U/ml penicillin and 100 μg/ml streptomycin (Gibco). B 16F10 mouse melanoma cells were purchased from ATCC, and were grown in Dulbecco's Modified Eagle's Medium (DMEM; WelGENE) supplemented with 10% FBS (WelGENE) and penicillin/streptomycin (100 U/ml; Gibco). Sk-Mel-28 human melanoma cells (from ATCC) were grown and maintained in RPMI-1640 medium, containing 10% FBS (WelGENE), and 100 U/ml penicillin and 100 μg/ml streptomycin (Gibco). The mouse prostate cancer cells (TRAMP-C2) were obtained from the American Type Culture Collection (ATCC) and cultured in Dulbecco's Modified Eagle's Medium (DMEM; WelGENE) containing penicillin and streptomycin (Gibco) and supplemented with 10% FBS (WelGENE). The human prostate cancer cell line (PC3), obtained from the American Type Culture Collection (ATCC), were cultured in RPMI 1640 medium containing 2.05 mM L-glutamine, 2 g/liter sodium bicarbonate and 2 g/liter glucose (WelGENE) together with 10% FBS (WelGENE), 100 U/ml penicillin and 100 μg/ml streptomycin (Gibco) at 37° C. in a humidified 5% CO2 atmosphere.

1-3. Animal Study.

BALB/c and C57BL/6 (B6) wild-type mice were purchased from DBL. For the subcutaneous tumor model of melanoma and prostate cancer, CT26 (3×10⁵ cells/mouse), B16F10 (1×10⁶ cells/mouse) and TRAMP-C2 cells (1×10⁶ cells/mouse) were mixed with Matrigel matrix (Corning) and inoculated subcutaneously into the right flank of the mice, and 4T1 (1×10⁵ cells/mouse) cells mixed with Matrigel matrix and inoculated into a 4^{th} mammary fat pad of the mice. TAMpepK, and MpepK peptides (200 nmol/kg) were injected intraperitoneally every 3 days, beginning at day 7 after tumor inoculation, and tumor volume was measured by electronic caliper. All tumor tissues were harvested after the end of the study and tumor weight was measured by an electronic balance.

For lung fibrosis mouse model, C57BL/6 (B6) wild-type mice were lightly anesthetized with 2.5% isoflurane and administered bleomycin (BLM, 2 mg/kg) via oropharyngeal aspiration (OA) using a micropipette. After 14 days, the mice were intraperitoneally injected with MpepK (200 nmol/kg) every other day. The animal studies were approved by the Institutional Animal Care and Use Committee of Kyung Hee University (KHUASP(SE)-20-530 for melanoma and 20-382 for prostate cancer). All animals were maintained in a specific pathogen-free environment on a 12-h light/dark cycle with free access to food and water. Nesting sheets were used for enrichment. After the experiments were terminated, all mice were euthanized using isoflurane and cervical dislocation.

1-4. Macrophage Differentiation.

THP-1 monocytes were differentiated into macrophages by 24 h incubation with 100 nM phorbol 12-myristate 13-acetate (PMA, Sigma) followed by 24 h incubation in RPMI medium (Invitrogen). Macrophages were polarized in M1 macrophages (M1) by incubation with 20 ng/ml of IFN-γ (Prospec) and 100 ng/ml of LPS (Sigma). Macrophage M2 polarization(M2) was obtained by incubation with 20 ng/ml of interleukin (IL) 4 (Prospec) and 20 ng/ml of interleukin 13 (Prospec). M2-like tumor-associated macrophages were polarized by incubation with 20% conditioned medium of PC3 cells.

1-5. Preparation of Conditioned Medium.

To obtain conditioned media of tumor (TCM), PC3 cells were seeded at 2×10⁵ cells/well in culture medium in 24-well plates (Corning Inc). After 24 hours, the medium was changed to serum-free RPMI1640 medium and the cells were incubated for 24 hours. For conditioned media of macrophages, THP-1 cells were seeded at 2×10⁵ cells/well in culture medium in 24-well plates (Corning Inc) and incubated with 100 nM PMA for 24 h. Cells were polarized into M0, M1, and M2 macrophages or TAM macrophages by TCM and changed to serum-free RPMI1640 medium. After 24 hours, the medium was changed to serum-free RPMI1640 medium and the cells were incubated for 24 hours. Supernatants were harvested and clarified with syringe filters (0.2 μm, Milipore). Supernatants of PC3 cells were named tumor-conditioned medium (TCM).

1-6. Flow Cytometry Analysis.

THP-1 cells were differentiated into macrophages by a 24 h incubation with 100 nM PMA and polarized in M2 macrophages by incubation with 20 ng/ml of IL-4 and 20 ng/ml of IL-13 for 72 h. Polarized cells were treated with 50 nM TAMpep and fragments of TAMpep or Mpep and alanine library of Mpep conjugated with FITC for 1 h. To test change of macrophage population in melanoma tissue, the single cells were isolated from tumor tissue through a 40 μm nylon mesh strainer after dissociation by DNase I (1 U/mL) and collagenase D (1 mg/ml). Cells were detected on BD FACSCalibur and BD FACSCantoII instruments and analyzed by FlowJo software.

1-7. Cell Viability Tests.

THP-1 cells were differentiated into macrophages by 24 h incubation with 100 nM PMA and polarized in M2 macrophages by incubation with 20 ng/ml of IL-4 and 20 ng/ml of IL-13 for 72 h. Polarized cells were treated with increasing concentrations of TAMpep and fragments of TAMpep (0.05-20 μM) for 24 h. Cell viability was analyzed using the CCK-8 assay: CCK-8 reagent (Enzo Life Sciences) was added to each well; incubation was continued for 2 hours, and absorbance was measured at 450 nm with a microplate reader (Molecular Devices).

1-8. Hemolytic Activity Assay.

Mouse blood samples were collected in tubes containing heparin as an anticoagulant and stored at 4 ° C. before use. Whole blood sample were centrifuged at 1,500×g for 5 min and the resulting plasma fraction was removed from the samples. The pellets were washed with an equal volume of saline, mixing by inversion. The centrifuging and washing steps were repeated 5 times. Red blood cells were counted by a hemocytometer and adjusted to -5×10⁷ cells/mL. Red blood cells were then incubated at 37° C. for 1 h in 1% Triton X-100 (positive control), in PBS (blank), or with increasing concentrations of TAMpep and Mpep (0.1-50 μM) were evaluated. The samples were then centrifuged at 10,000 g for 5 min, the supernatant was separated from the pellet, and its absorbance measured at 570 nm. The relative optical density compared to that of the suspension treated with 1% Triton X-100 was defined as the percentage of hemolysis.

1-9. ELISA Assays.

To test for polarization of human macrophages, THP-1 cells were seeded at 2×10⁵ cells/well in culture medium in 24-well plates (Corning Inc) and incubated with 100 nM PMA for 24 h. Macrophages were polarized in M1 macrophages by incubation with 20 ng/ml of IFN-γ and 100 ng/ml of LPS and M2 macrophages by incubation with 20 ng/ml of IL-4 and 20 ng/ml of IL-13. After differentiation, the supernatant of macrophages was collected. Markers of M2 macrophages such as IL-10 and TGF-β, and M1 macrophages such as IL-12 and CXCL10 were measured by ELISA kits according to the manufacturer's instructions (BD Biosciences Inc.).

1-10. Immunofluorescence Assay.

THP-1 cells were seeded on cover glasses in 24-well plates and differentiated into M0, M1 and M2 macrophages. Cells were treated with 1 μM TAMpepK and MpepK for 1 h and incubated for 24 h after remove of peptides. Cells were washed, fixed with 4% paraformaldehyde for 10 minutes at −20° C. and blocked with 0.1% normal goat serum for 1 hour. The cover glasses were then incubated with anti-caspase-3 antibody (1:50, rabbit polyclonal, Abcam) overnight at 4° C., and then washed and stained with Alexa 594-labelled goat anti-rabbit lgG (1:500, Invitrogen) at 37° C. for 1 hr. The cover glasses were mounted in Vectashield mounting medium (Vector Laboratories) with DAPI to visualize nuclei. Images photographed by fluorescence microscope (Leica).

1-11. Real-Time Quantitative PCR.

Total RNA was extracted using Easy-Blue reagent. Concentrations of RNA were determined and quantified by measuring absorbance at 260 and 280 nm with a spectrophotometer. Complementary DNA (cDNA) was synthesized from total RNA using a Maxime RT PreMix kit (iNtRON). Real-time PCR analysis was performed with SYBR Green Master Mix. PCR conditions were forty-five cycles at 95° C. for 5 min, followed by 95° C. for 10 sec, 60° C. for 10 sec and 72° C. for 10 sec. mRNA expression were quantified in triplicate. Data were measured with CFX Software (Bio-Rad). GAPDH and β-actin were used as internal controls.

1-12. Western Blot Analysis.

Cells were harvested and lysed in PRO-PREP protein extraction solution (iNtRON, Bio Inc, Sungnam, Korea). Protein concentrations were measured with a Bradford Protein Assay Reagent kit (Bio-Rad, Richmond, CA, USA). Proteins were fractionated by 10% SDS-polyacrylamide gels electrophoresis (PAGE), and transferred onto polyvinylidene difluoride (PVDF) membranes. These were incubated with anti-arginase 1, anti-CD206, anti-caspase 3, anti-E-cadherin, anti-fibronectin, anti-PCNA, anti-TGF-β, anti-MMP9, and anti-β-actin Ab as primary antibodies. Goat anti-rabbit horseradish peroxidase-conjugated IgG or goat anti-mouse horseradish peroxidase-conjugated IgG (Abcam, Cambridge, Mass., USA) served as secondary antibodies. Protein bands were detected with a chemiluminescence reagent kit (SurModics).

1-13. Wound Healing Assay.

Migration of prostate cancer and melanoma cells was assessed with wound healing assays. PC3 and Sk-Mel-28 cells were seeded at 2×10⁵ cells/well in 24-well plates and cultured in RPMI1640 with 10% FBS. When the cells reached confluence, they were wounded by scraping across the surface of the well with a sterile micropipette tip. The cells were immediately washed and the wells were filled with serum-free medium or 20% conditioned media of M0, M1, M2, and M-TCM without or with TAMpepK or MpepK and incubated or 24 hr. Before and after incubation, at least five different fields of the wounded area of each sample were photographed using an inverted microscope (Olympus). Wound areas were measured with ImageJ software (NCI, Bethesda, Md., USA). The percent of each wounded area filled by cell migration was calculated as: (mean wounded breadth—mean remaining breadth)/mean wounded breadth×100.

1-14. Invasion Assay.

The invasiveness of prostate cancer cells treated with conditioned media of macrophages was tested according to the manufacturer's instructions for the invasion assay (Corning Inc.) with slight modifications. Briefly, invasiveness was assessed using 24-well plates fitted with polycarbonate 8-μm pore membrane inserts (Corning Inc.) pre-coated with Matrigel (200-300 μg/mL) for 2 hours at 37° C. The lower wells were filled with 350 μL of serum-free RPMI1640 medium or 20% conditioned medium (conditioned media of M0, M1, M2, and M-TCM without or with TAMpepK or MpepK). The upper wells were filled with 200 μL PC3 cells (5×10⁴ cells/well) in serum-free medium. The plates were incubated for 24 hours. The cells were then fixed in methanol and stained with Giemsa. Five randomly selected fields per membrane were counted under a light microscope (Olympus). The invasion index was calculated from the number of cells that migrated in response to conditioned medium compared with the control without conditioned medium.

1-15. H&E Staining.

The lung tissues of lung fibrosis mouse model were fixed in 10% neutral buffered formalin and embedded in paraffin. The paraffin-embedded tissue samples were sectioned into 5 μm slices, then deparaffinized, and stained with H&E to investigate the degree of lung tissue fibrosis. The sections were examined and evaluated randomly using standard light microscopy (Olympus).

### Example 2. Results

2-1. Polarization of THP-1-Derived Macrophages.

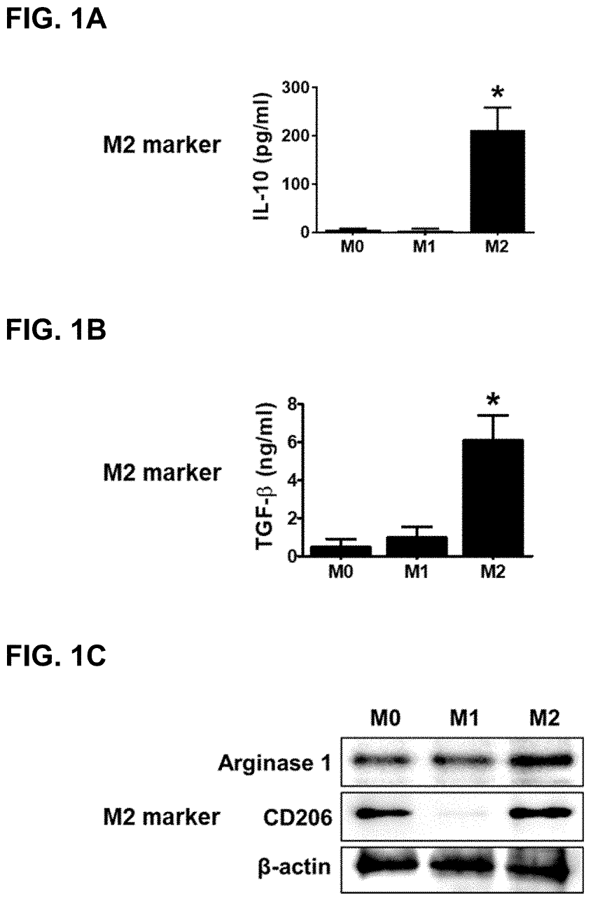

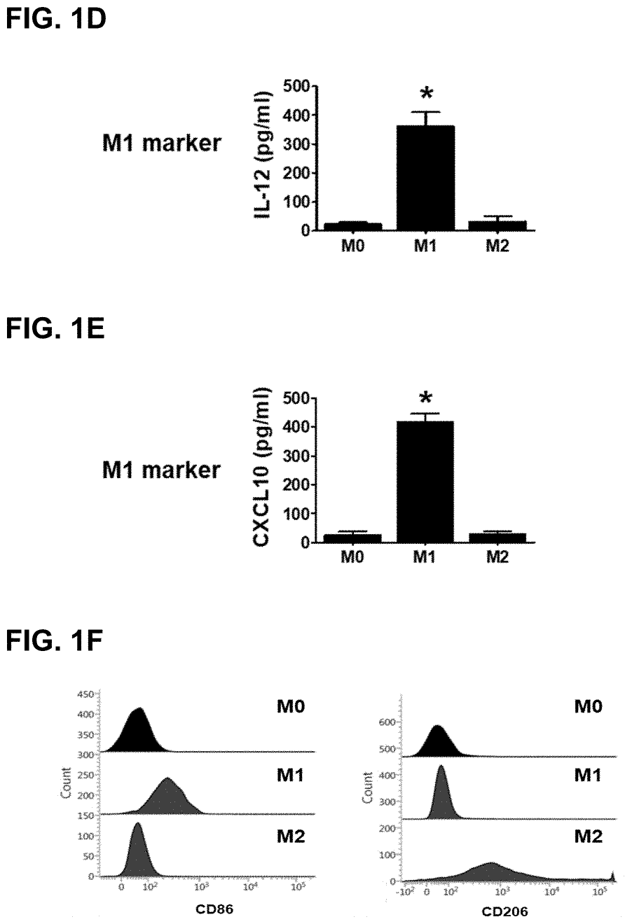

To polarize into M1 or M2 macrophages, THP-1 cells were treated with PMA for M0 macrophages, and then incubated with LPS and IFN-γ for M1 macrophages and IL-4 and IL-13 for M2 macrophages. Polarization of macrophages was assessed by markers of M1, such as IL-12, CXCL10, and CD86, and M2, such as IL-10, TGF-β, arginase 1, and CD206. Macrophages treated with LPS and IFN-γ showed increased M1 markers (FIGS. 1D, 1E, and 1F) and macrophages treated with IL-4 and IL-13 showed increased M2 markers compared to M0 (FIGS. 1A, 1B, 1C, and 1F).

Thus, the polarized macrophages could be used for further study assessing efficacy of TAMpepK or MpepK targeting M2 macrophages.

2-2. Affinity of TAMpep Fragments in THP-1-Derived M2 Macrophages.

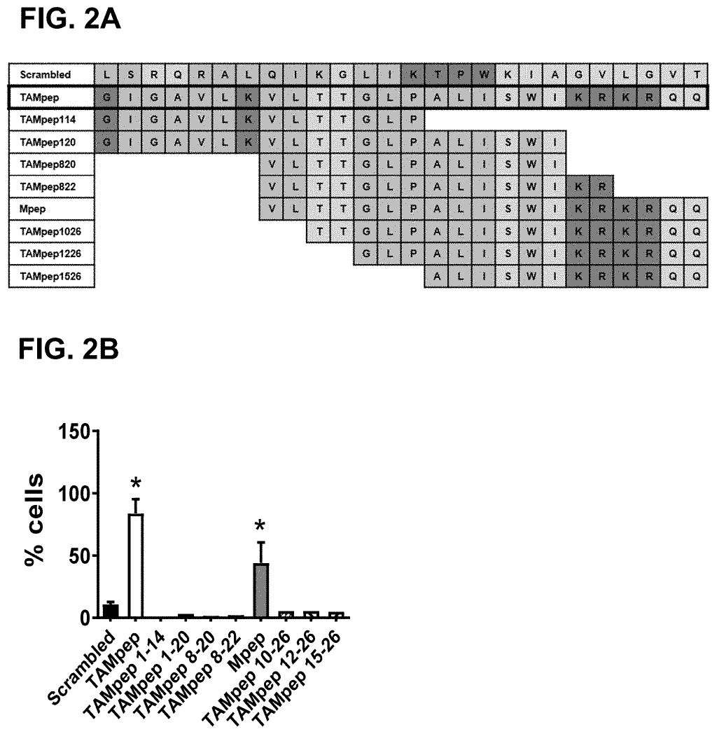

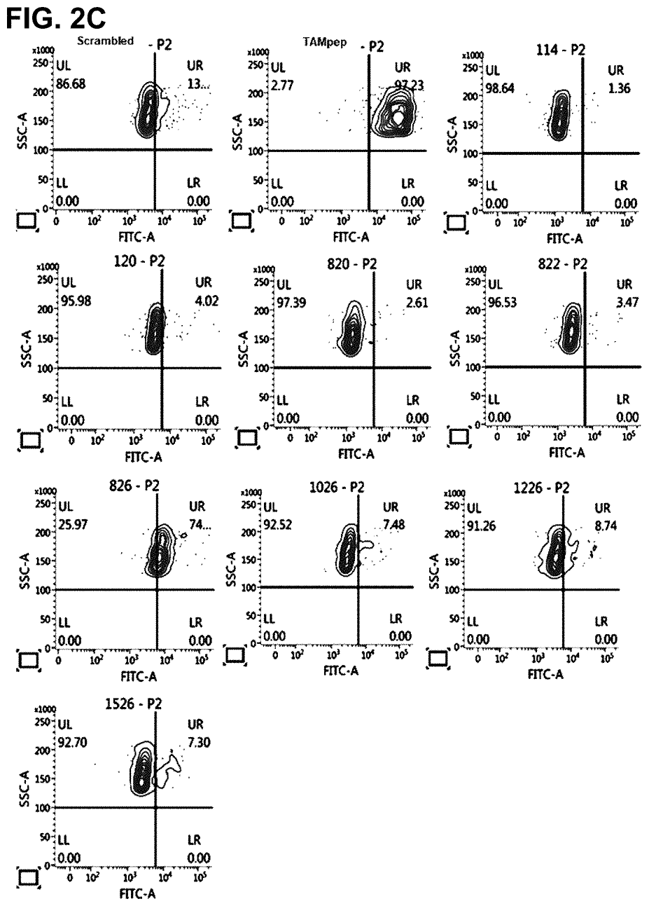

To determine major amino site of TAMpep binding to M2 macrophages, the affinity test was conducted by using TAMpep and fragments of TAMpep (amino acid sequence, FIG. 2A) conjugated with FITC in THP-1-derived M2 macrophages. TAMpep (including 26 amino acids) was showed high affinity of over 90% and Mpep (removed 7 amino acids from C terminus) was showed second highest affinity as over 45% in M2 macrophages. On the other hand, fragments of TAMpep (removed over 10 amino acids from C terminus or over 4 amino acids from N terminus) were indicated low affinity compared with scrampled peptide of 26 amino acids (FIGS. 2B and 2C). Thus, these results suggested that 4-6 amino acids of N terminus are amino site to play a key role in affinity of TAMpep to M2 macrophages.

2-3. Cytotoxicity of TAMpep Fragments in THP-1-Dervied M2 Macrophages.

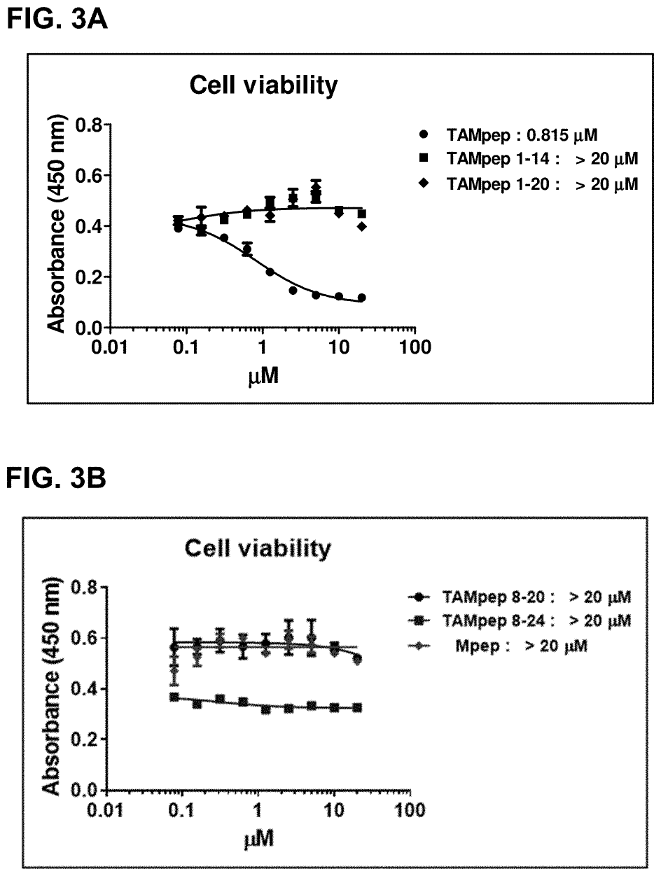

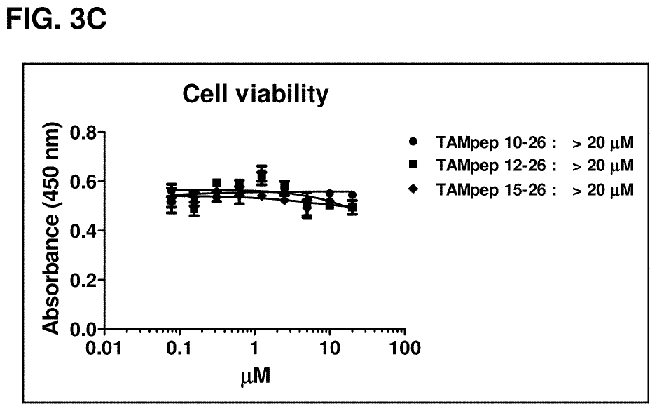

The TAMpep of 26 amino acids has cytotoxicity and can cause side effects to normal cells or tissues when used as a drug carrier. Therefore, a new sequence peptide having features of high affinity and low cytotoxicity to M2 macrophages was needed. Various TAMpep fragments were tested in a cytotoxicity assay in THP-1-derived M2 macrophages. TAMpep showed a high cytotoxic value of 0.815 μM IC50 while other peptide fragments did not show cytotoxic effect in M2 macrophages (FIGS. 3A-3C). In particular, Mpep showed high affinity and low cytotoxicity in M2 macrophages and thus it was expected to be an optimal drug carrier.

2-4. Hemolysis of TAMpep and Mpep.

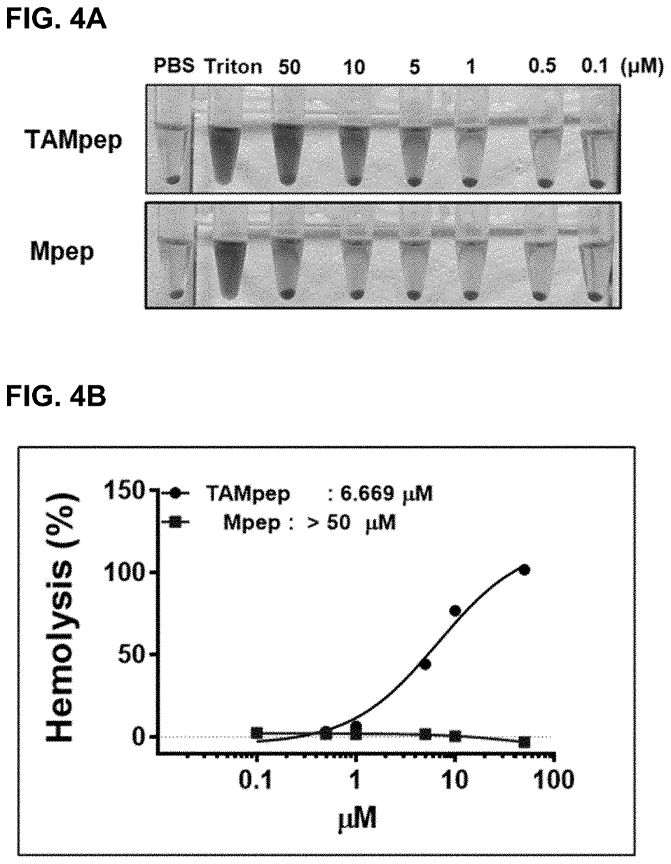

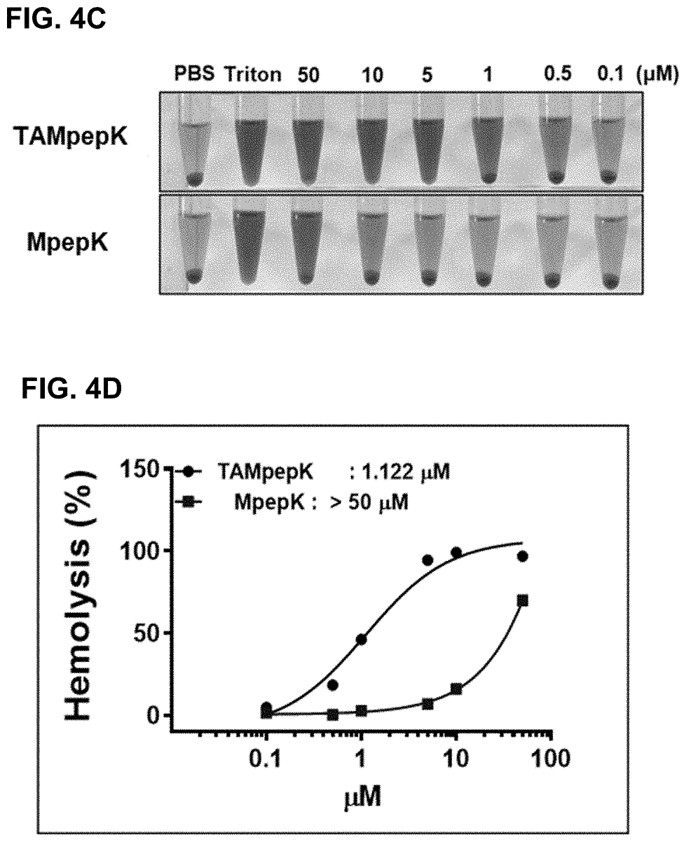

The hemolytic effect can cause serious side effects and is one of the factors limiting the dosage of a drug. To determine hemolysis of TAMpep and Mpep, peptides were treated with increasing concentrations (0.1-50 μM) in mouse RBC. TAMpep showed 6.669 μM at IC50 and while Mpep showed >50 μM at IC50 (FIGS. 4A and 4B). In addition, TAMpep and Mpep conjugated dKLA showed 1.122 μM and >50 μM at IC50, respectively (FIGS. 4C and 4D). Thus, Mpep can developed as a safe drug with fewer side effects.

2-5. Affinity of TAMpep and Mpep in THP-1-Derived Macrophages.

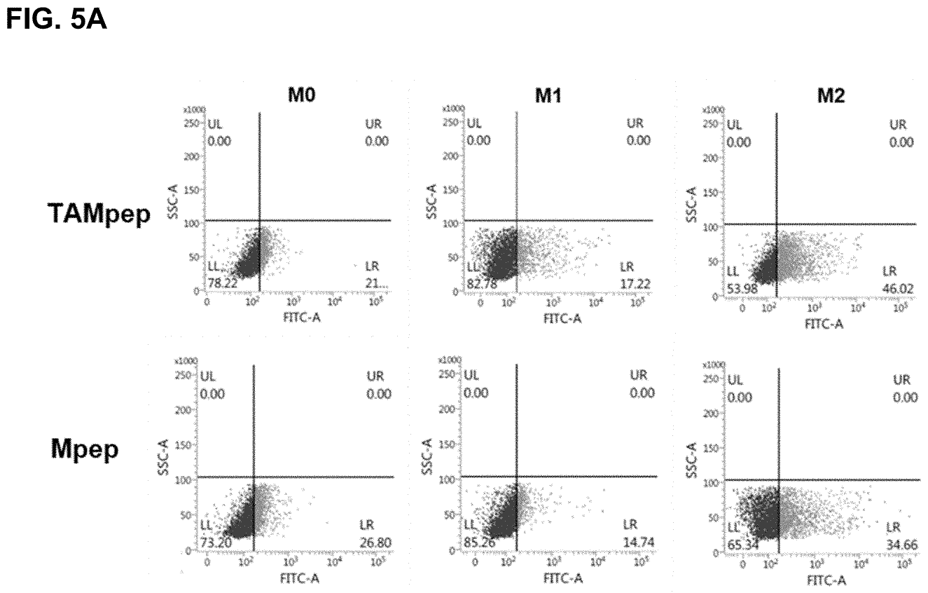

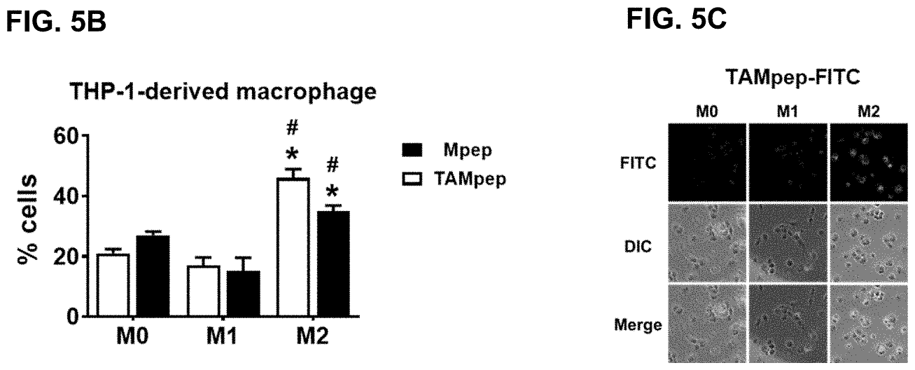

To compare whether TAMpep and Mpep adhere more specifically to M2 macrophages among subtypes of macrophages, the peptides conjugated with FITC were treated with M0, M1, and M2 macrophages polarized from THP-1 cells and analyzed by FACs. Both TAMpep and Mpep showed significantly more high affinity in M2 macrophages compared to M0 and M1 macrophages (FIGS. 5A and 5B). Additionally, TAMpep showed high affinity in M2 macrophages by immunofluorescence microscopy (FIG. 5C).

2-6. Cytotoxicity of TAMpepK and MpepK in THP-1-Derived Macrophages.

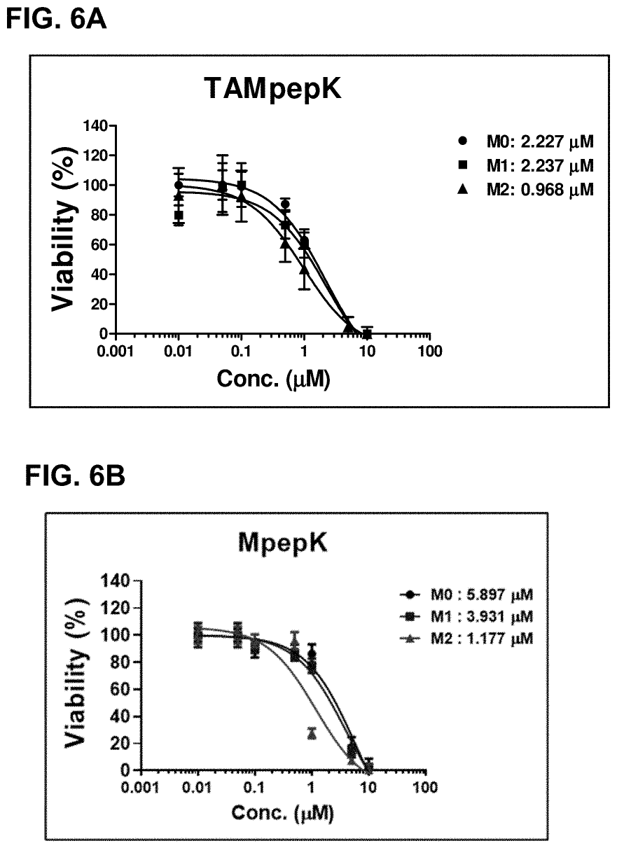

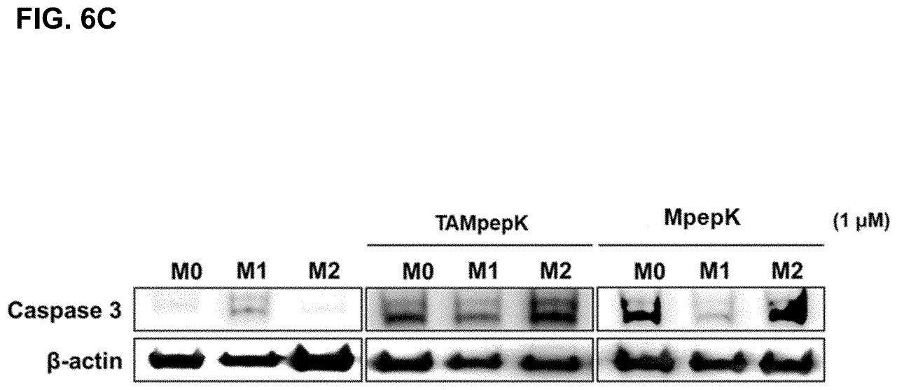

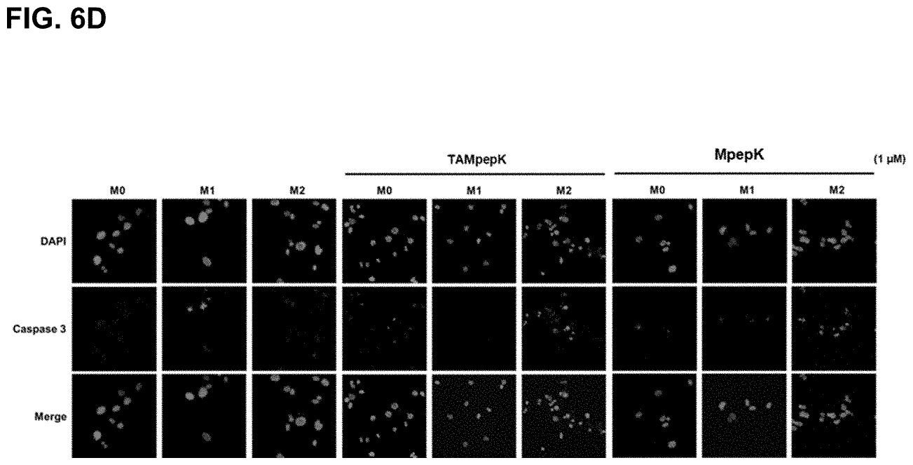

To assess whether TAMpep and Mpep conjugated dKLA induce selective apoptosis, M2 macrophages were treated with increasing concentration of TAMpepK or MpepK (0.01 -10 μM). As a result, TAMpepK and MpepK induced apoptosis in M2 macrophages compared to M0 and M1 macrophages (FIGS. 6A and 6B). Furthermore, expression of caspase-3, which is related with apoptosis, was increased in M2 macrophages compared to other subtype macrophages (FIGS. 6C and 6D).

2-7. Affinity of Mpep by Alanine Library in THP-1-Derived Macrophages.

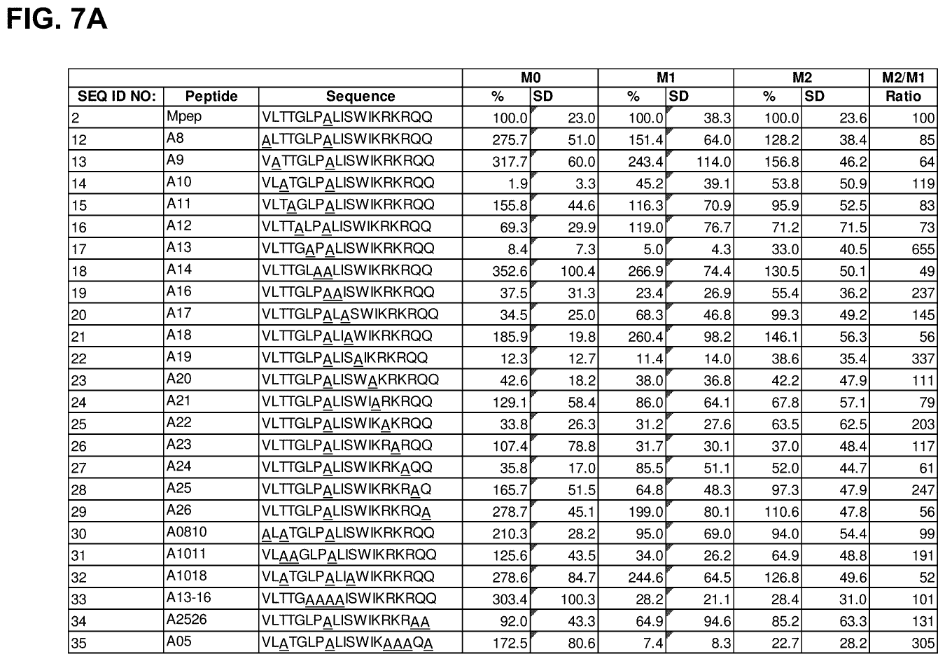

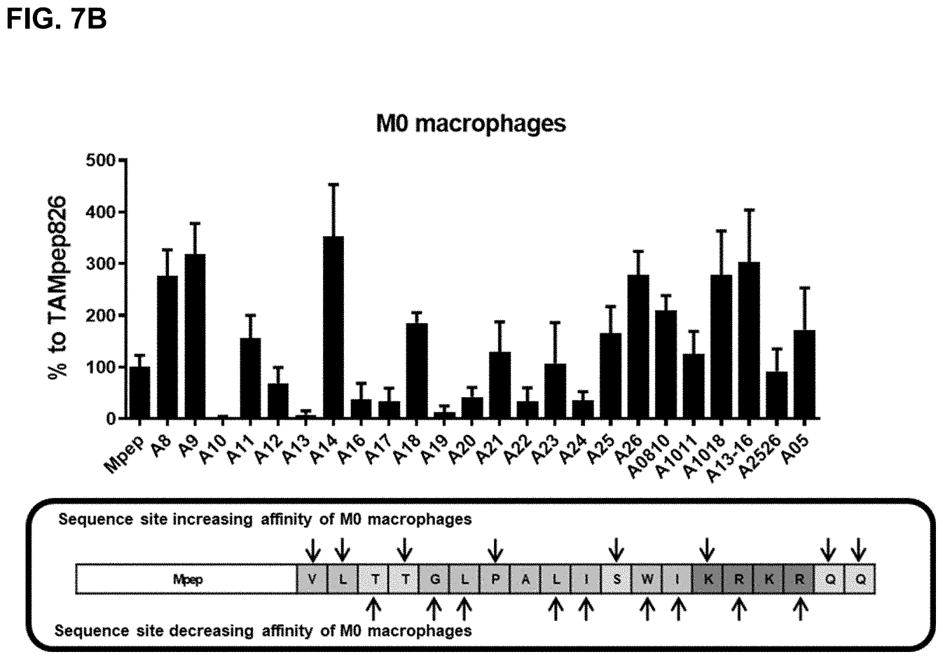

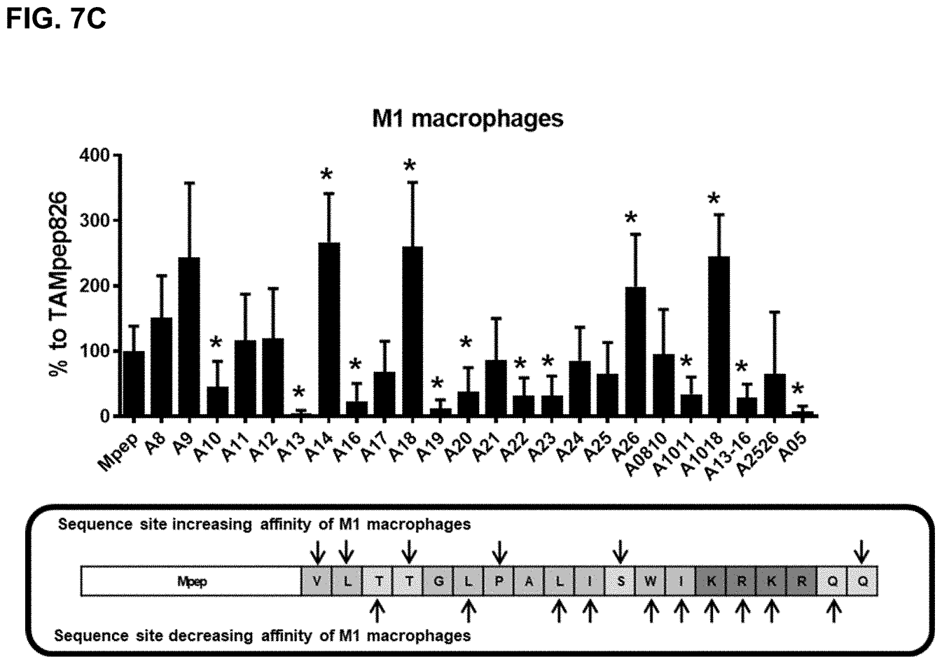

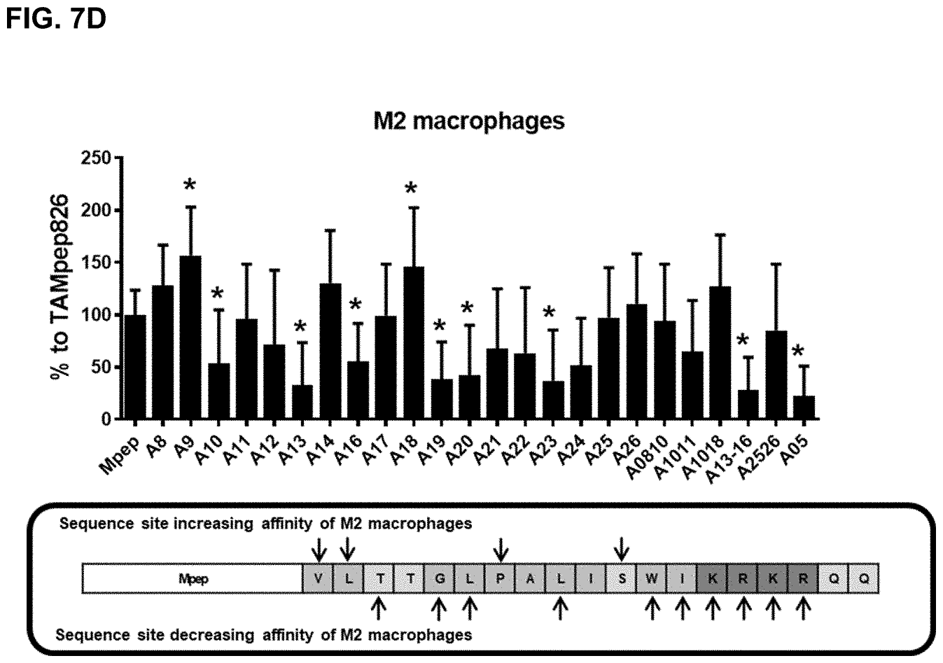

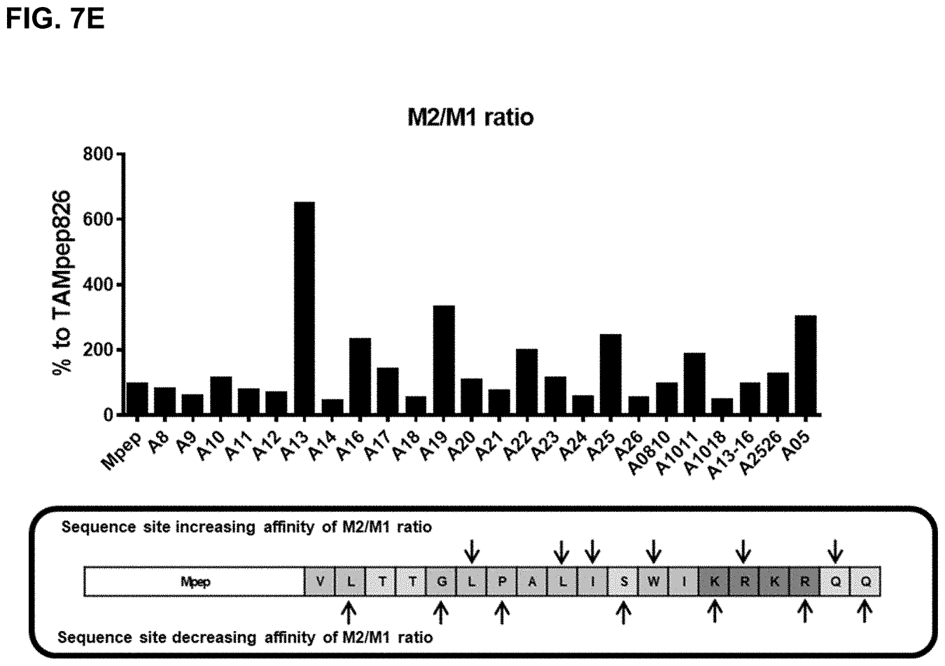

To find the key amino acid sequence important in the adhesion ability of Mpep in M2 macrophages, the alanine-substituted library of Mpep was used. In M2 macrophages, affinity of peptides was decreased when alanine was substituted in the third T (threonine), 6th L (leucine), ninth L (leucine), twelfth W (tryptophan), thirteenth I (isoleucine), sixteenth K (lysine) and 17th R (arginine). In addition, affinity of peptides was reduced in the peptides (A13-16 and A05) substituted for the sixth L (leucine) through the ninth L (leucine) and the third T (threonine), the fifteenth K (lysine), the sixteenth R (arginine), the seventeenth K (lysine), and the nineteenth Q (glutamine). On the other hand, the peptides (A9 and A18) substituted the second L (leucine) and eleventh S (serine) showed increased affinity in M2 macrophages FIGS. 7A-7E.

2-8. Cytotoxicity of TAMpepK in M2 Macrophages and Human Melanoma Cells.

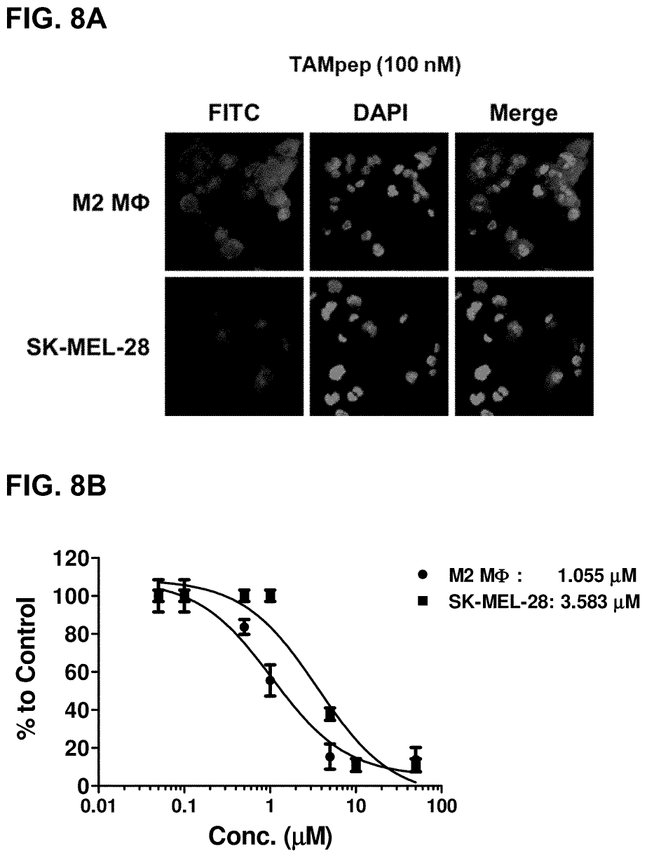

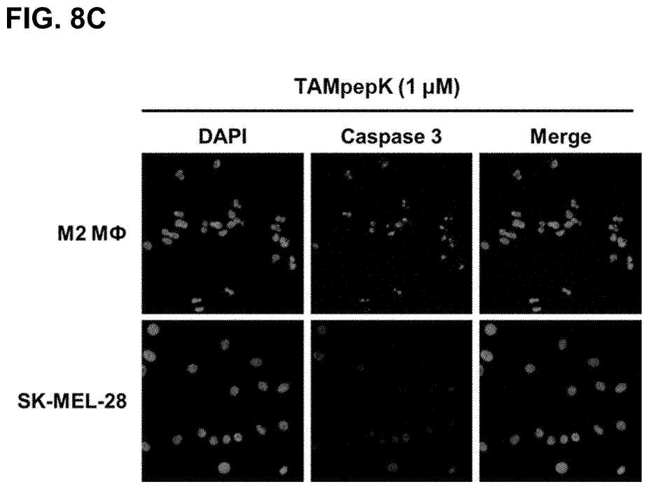

To determine whether TAMpepK induces more apoptosis and binding to M2 macrophages than melanoma cells, THP-1-derived M2 macrophages and Sk-Me1-28 cells were treated with TAMpep (FIG. 8A) or TAMpepK (FIG. 8C). TAMpepK showed low IC50 value (1.055 μM) in M2 macrophages compared to melanoma cells (IC50: 3.583 μM) and expression of caspase-3 was also increased in M2 macrophages compared to melanoma cells (FIG. 8C). Thus, these findings suggest that TAMpep binds selectively to M2 macrophages and induces apoptosis.

2-9. Proliferation and Migration in Melanoma Cells by Conditioned Medium of M2 Macrophages Treated with TAMpepK.

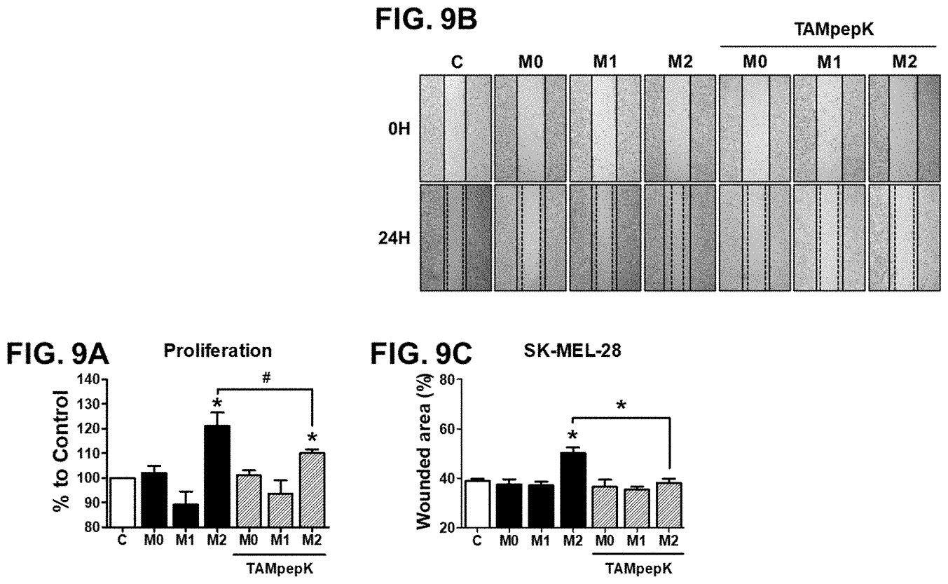

To test whether TAMpepK inhibit the proliferation and migration of melanoma cells induced by M2 macrophages, conditioned medium of M0, M1 and M2 macrophages pretreated without or with TAMpepK (1 μM) and the conditioned medium treated in melanoma cells were prepared. Proliferation of melanoma cells was increased by conditioned medium of M2 macrophages while inhibited in conditioned medium of M2 macrophages pretreated with TAMpepK (FIG. 9A). Moreover, conditioned medium of M2 macrophage pretreated with TAMpepK inhibited migration of melanoma cells but migration was increased by conditioned medium of M2 macrophages (FIGS. 9B and 9C). Thus, TAMpepK inhibits proliferation and migration of melanoma cells by inducing apoptosis of M2 macrophages.

2-10. Anti-Cancer Effect of TAMpepK in Mouse Model of Melanoma.

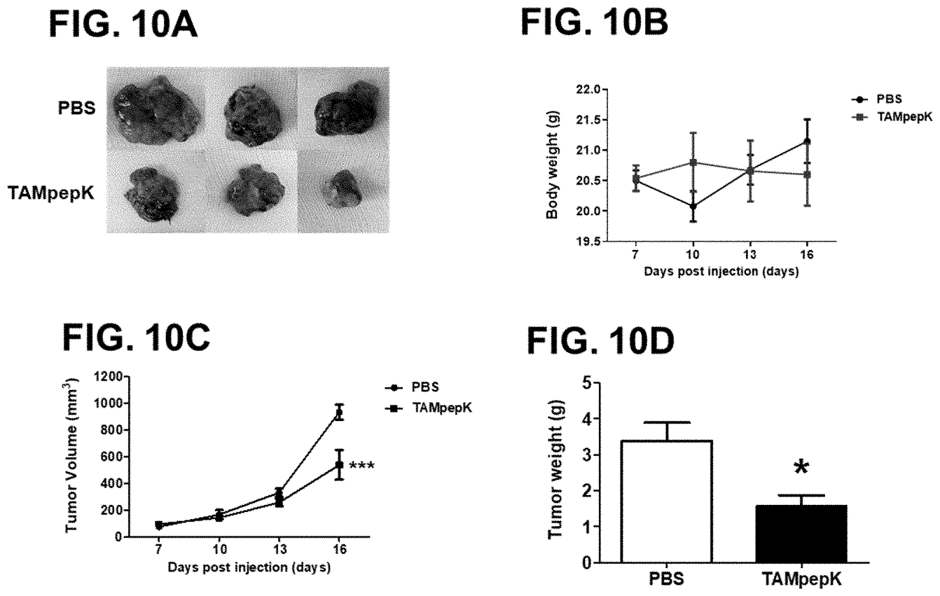

To assess the anti-cancer effect of TAMpepK in vivo, murine melanoma cells (B16F10 cell line) were injected subcutaneously in the right flank of C57BL6J mice and TAMpepK was injected intraperitoneally every 3 days after a week. Mice treated with TAMpepK showed significantly reduced tumor volume and weight compared with the PBS group (FIGS. 10A, 10C, and 10D). On the other hand, the body weight of mice was not significantly changed between the PBS and TAMpepK groups (FIG. 10B).

2-11. Effect of TAMpepK Targeting M2-like TAMs in Mouse Model of Melanoma.

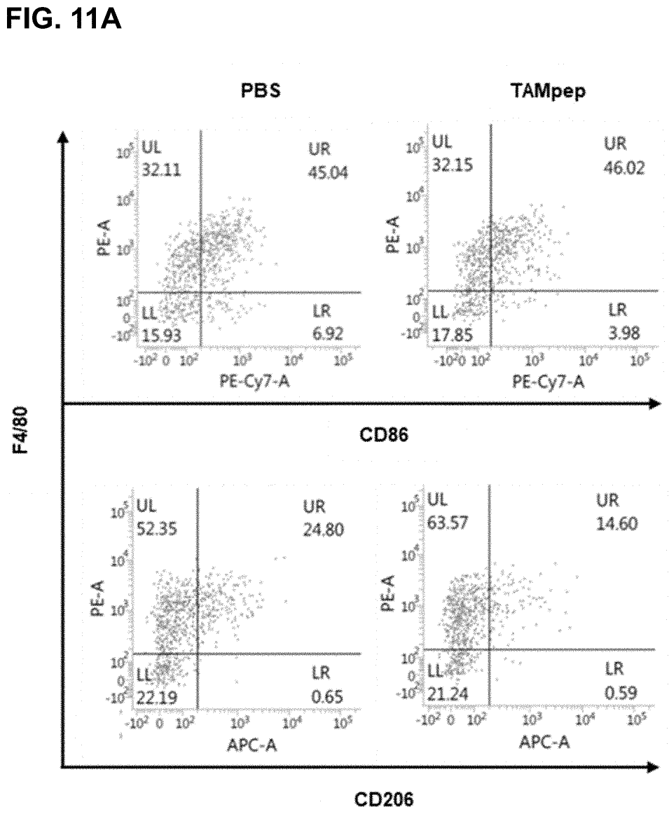

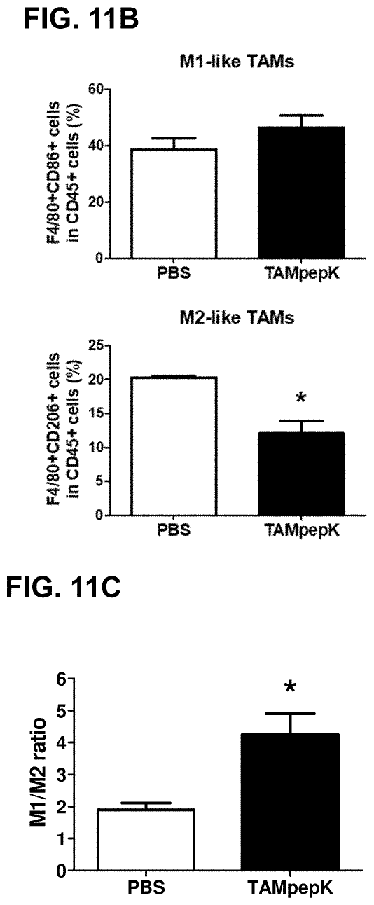

To determine whether TAMpepK reduces M2-like TAMs in mouse model of melanoma, macrophages were isolated from tumor tissues and analyzed by FACS. M2-like TAMs (F4.80+ and CD206+ cells) were reduced significantly in the TAMpepK group compared to the PBS group. However, M1-like TAMs (F4/80+ and CD86+ cells) did not a change between PBS and TAMpepK groups (FIGS. 11A and 11B). Additionally, the change in tumor microenvironment through the M1/M2 ratio was analyzed. TAMpepK group increased the rate of M1 macrophages by reducing M2 macrophages compared to the PBS group (FIG. 11C). Thus, these findings suggest that TAMpepK has an anti-cancer effect by targeting M2-like TAMs in melanoma model.

2-12. Anti-Cancer Effect of TAMpepK and MpepK in Mouse Model of Melanoma.

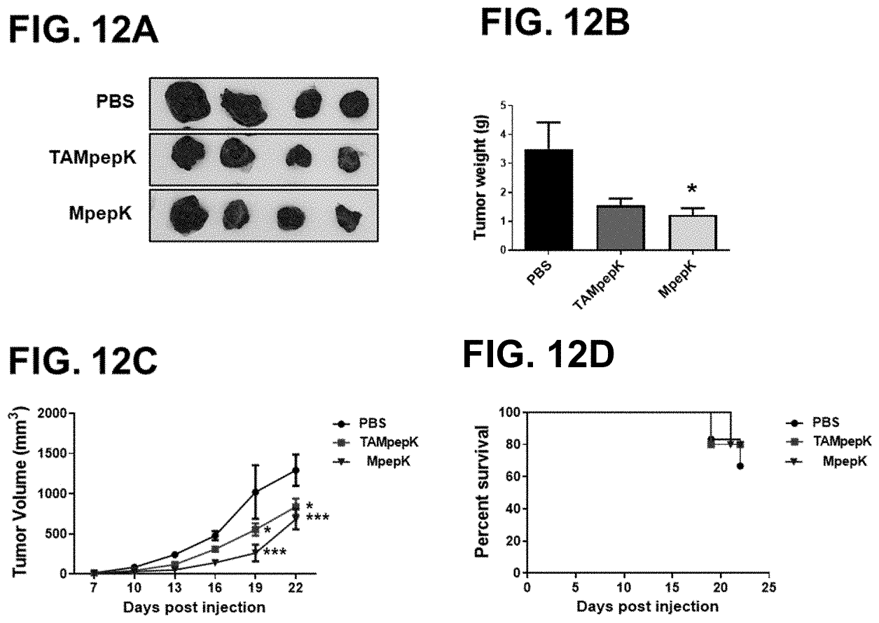

Anti-cancer effect of TAMpepK were shown in the above results. This study was done to determine the anti-cancer effect of MpepK in melanoma model. The photos of the tumors are shown in FIG. 12A. Tumor volume (FIG. 12C) and weight (FIG. 12B) were reduced in both TAMpepK and MpepK groups, and survival rate (FIG. 12D) was extended in the MpepK group compared to the PBS group.

2-13. Effect of TAMpepK and MpepK Targeting M2-like TAMs in Mouse Model of Melanoma.

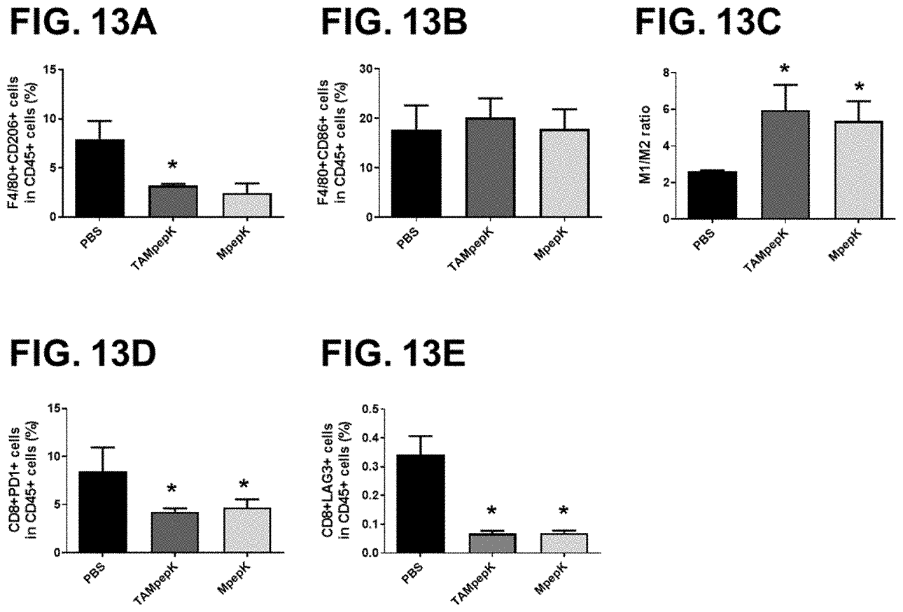

To determine whether MpepK induces a change of tumor microenvironment in melanoma, M1/M2 ratio of macrophages and CD8 exhaustion were analyzed by FACs. M2-like TAMs (F4.80+ and CD206+ cells) were reduced in TAMpepK and MpepK groups compare to the PBS group. However, M1-like TAMs (F4/80+ and CD86+ cells) showed no change in all group (FIGS. 13A and 13B). M1/M2 ratio was significantly increased in TAMpepK and MpepK groups compared to the PBS group (FIG. 13C). In addition, the exhaustion markers, such as PD-1 and LAGS, in CD8+ T cells was significantly reduced in the TAMpepK and MpepK groups compared to the PBS group (FIGS. 13D and 13E). Thus, these findings suggest that MpepK has an anti-cancer effect by targeting M2-like TAMs in a melanoma model.

2-14. Differentiation of THP-1-derived M2 macrophages by Conditioned Medium of Prostate Tumor Cells (TCM).

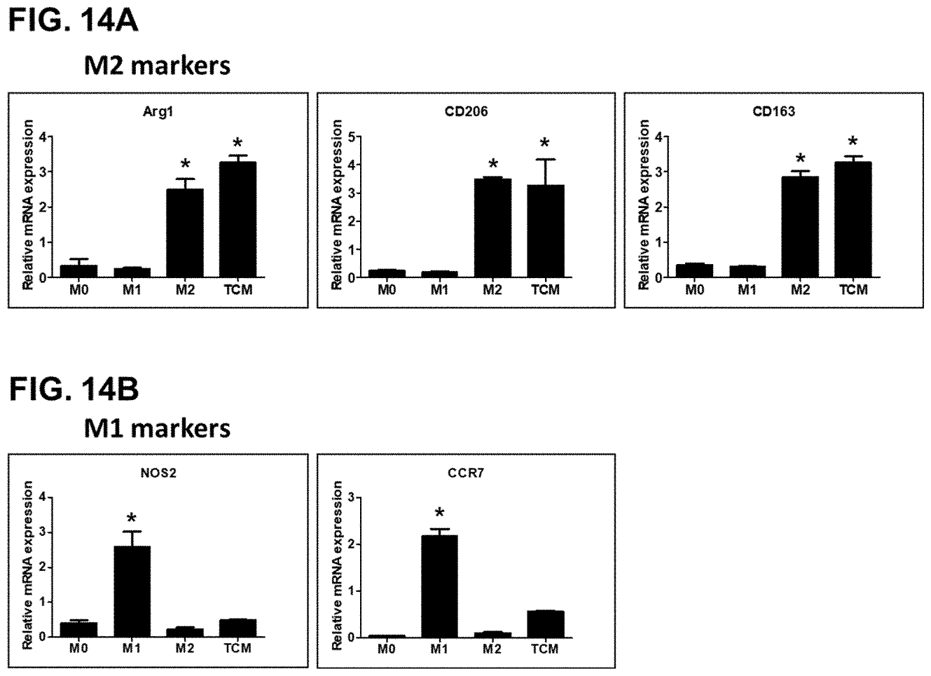

To determine polarization of M2 macrophages by conditioned medium of prostate cancer cells (TCM), THP-1-derived macrophages were incubated with TCM. TCM-treat macrophages showed increased mRNA expression of M2 markers such as arginase 1, CD206 and CD163 and showed decreased mRNA expression of M1 markers such as NOS2 and CCR7, compared with M0 macrophages (FIGS. 14A and 14B). Thus, this study showed induced polarization into M2-like TAMs in tumor microenvironment of prostate cancer.

2-15. Proliferation and Migration in Prostate Cancer Cells by Conditioned Medium of M2 macrophages.

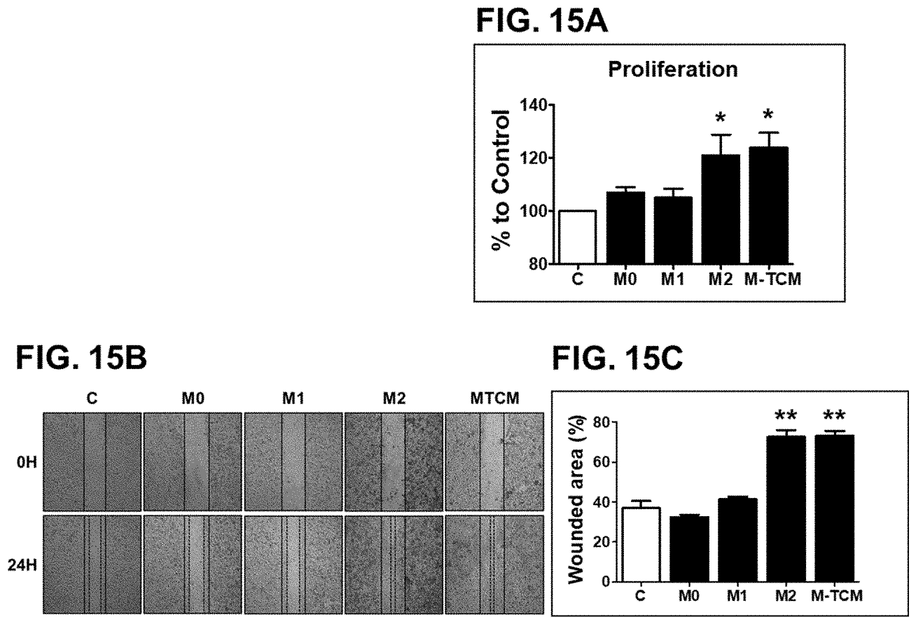

As shown in FIGS. 15A-15C, proliferation and migration of cancer cells were increased by THP-1-dervied M2 macrophages. M2 macrophages polarized by TCM induce proliferation and migration of prostate cancer cells were tested. Conditioned medium of macrophages treated with TCM (M-TCM) increased proliferation (FIG. 15A) and migration (FIGS. 15B and 15C) of prostate cancer cells, similar to conditioned medium of THP-1-derived M2 macrophages (FIGS. 15A-15C).

2-16. Cell viability of macrophages by TAMpepK or MpepK.

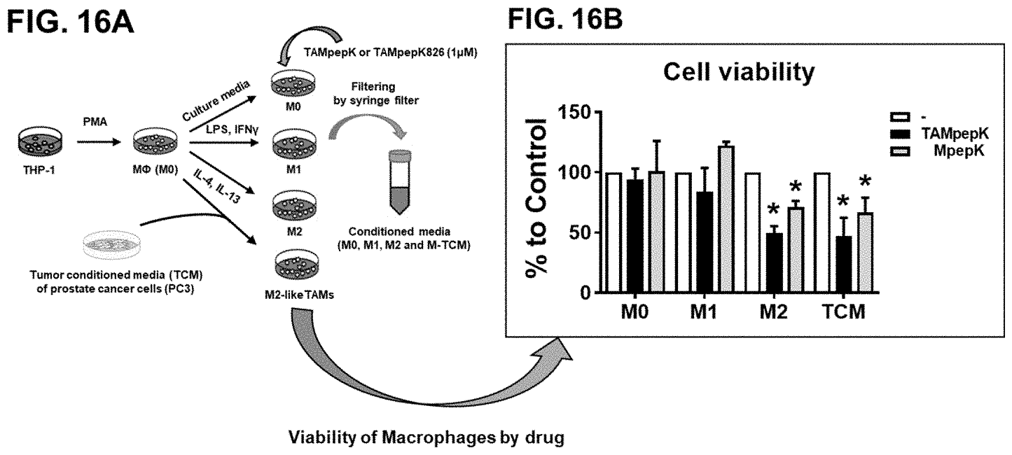

TAMpepK and MpepK induced apoptosis of M2 macrophages as shown in the above results. To assess whether TAMpepK and MpepK reduce cell viability of M2 macrophages differentiated by TCM, THP-1-derived macrophages were treated with TAMpepK and MpepK (1 μM). TAMpepK and MpepK induced apoptosis in macrophages treated with TCM, similar to M2 macrophages (FIG. 16B). Thus, this result suggests that TAMpepK and MpepK target M2 macrophages as well as macrophages induced by TCM.

2-17. Proliferation and migration in prostate cancer cells by conditioned medium of M2 macrophages treated with TAMpepK and MpepK.

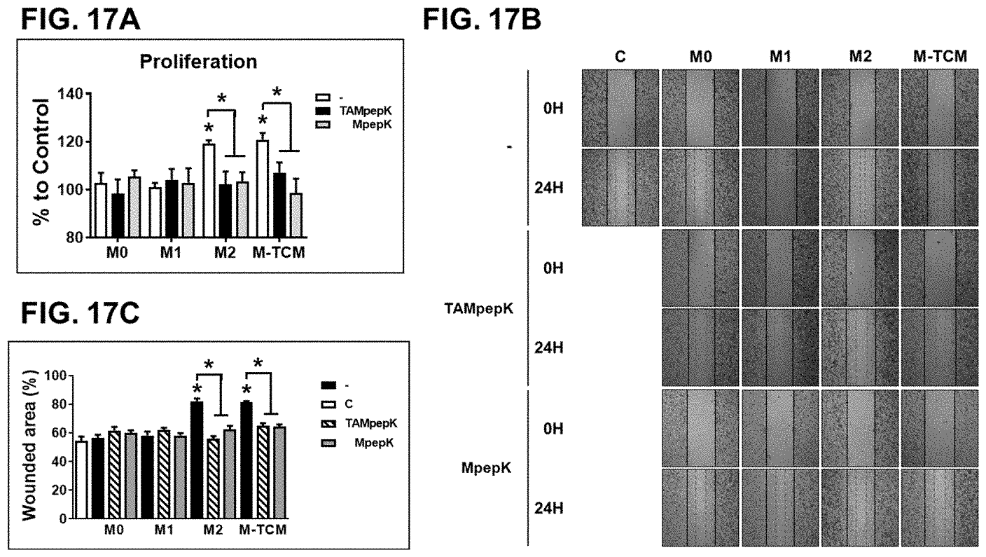

Conditioned media of M2 macrophages and M2-like TAMs induced by TCM increased proliferation and migration of prostate cancer cells (PC3 cells). However, conditioned media of M2 macrophages and M2-like TAMs pretreated with TAMpepK and MpepK were significantly reduced proliferation (FIG. 17A) and migration (FIGS. 17B and 17C) of PC3 cells compared to group of M2 macrophages or M2-like TAMs.

2-18. Invasion in Prostate Cancer Cells by Conditioned Medium of M2 Macrophages Ttreated with TAMpepK and MpepK.

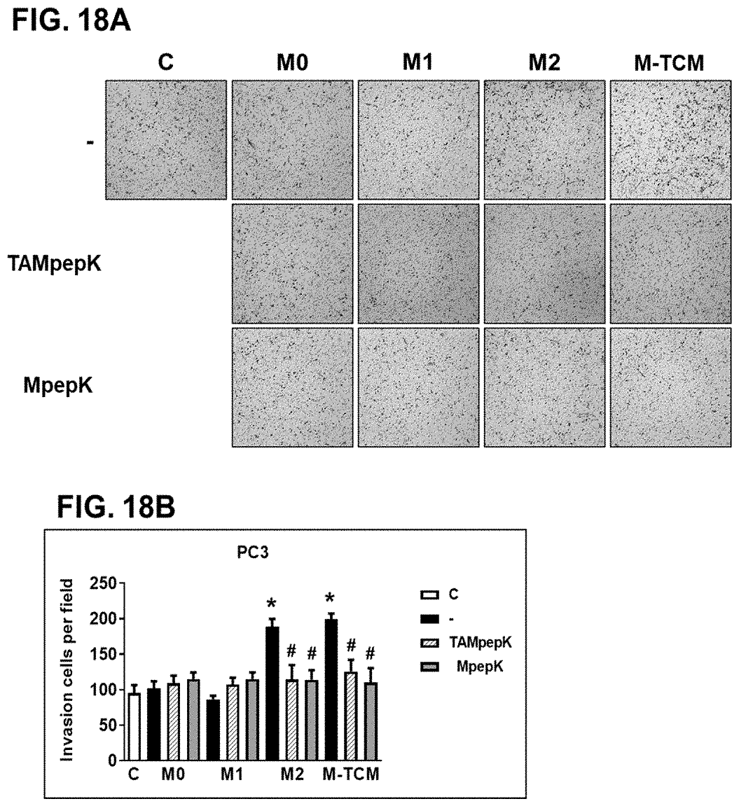

To determine to inhibit invasion of prostate cancer cells by TAMpepK and MpepK, PC3 cells were treated with conditioned medium of macrophages. Conditioned medium of M2 macrophages and M2-like TAMs induced by TCM were increased invasion of PC3 cells. However, conditioned medium of M2 macrophages and M2-like TAMs pretreated with TAMpepK and MpepK were significantly reduced invasion of PC3 cells compared to group of M2 macrophages or M2-like TAMs (FIGS. 18A and 18B).

2-19. Effect of TAMpepK and MpepK in Mouse Model of Prostate Cancer.

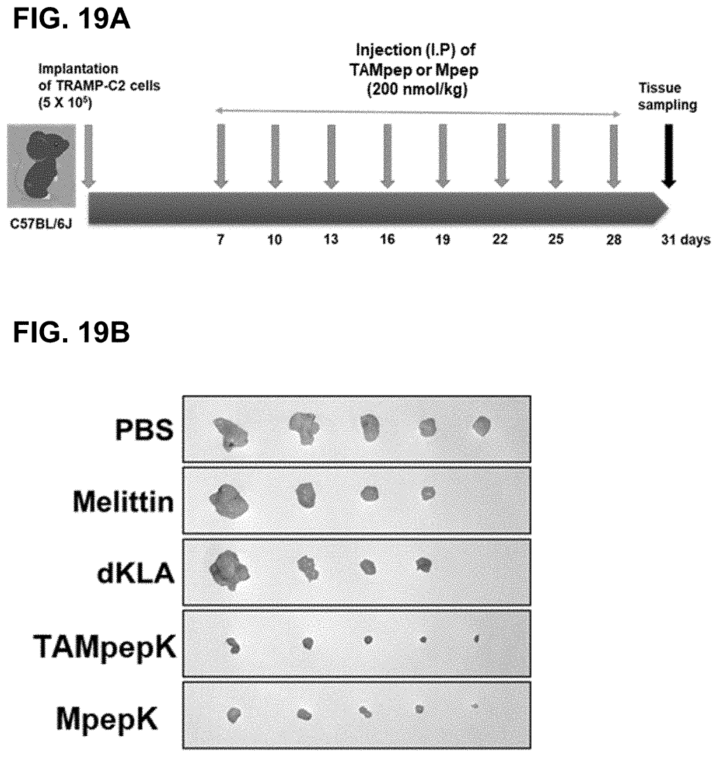

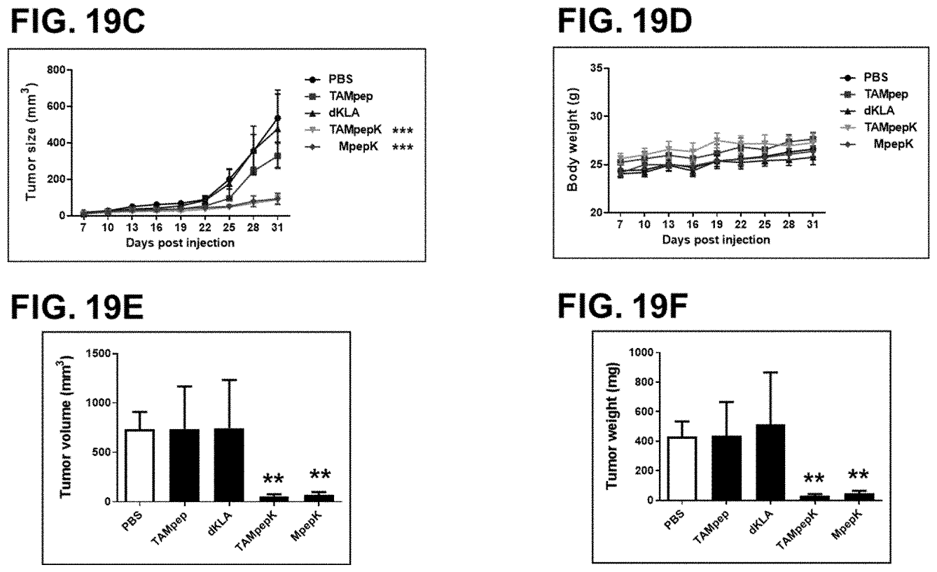

To assess anti-cancer effect of TAMpepK and MpepK in prostate cancer model, TRAMP-C2 cells were injected subcutaneously in right flank of C57BL6J mice and TAMpep, dKLA, TAMpepK and MpepK were injected intraperitoneally every 3 days after a week. Mice treated with TAMpepK and MpepK showed significantly reduced tumor volume and weight compared with PBS group (FIGS. 19B, 19C, 19E, and 19F). On the other hand, the body weight of mice did not significantly change between all groups (FIG. 19D).

2-20. Effect of TAMpepK and MpepK in Proliferation and EMT of Prostate Cancer Model.

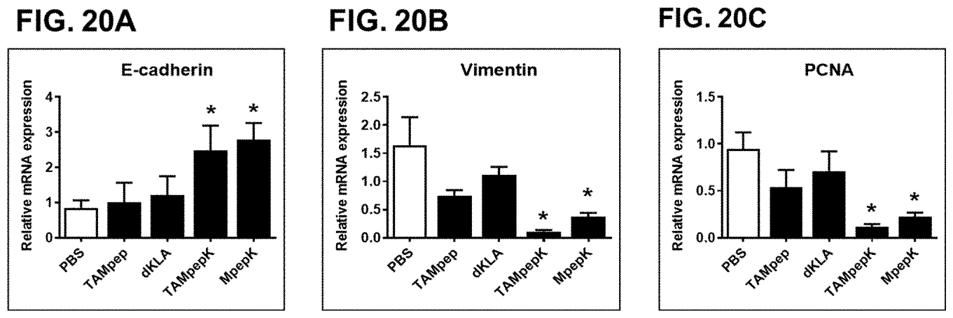

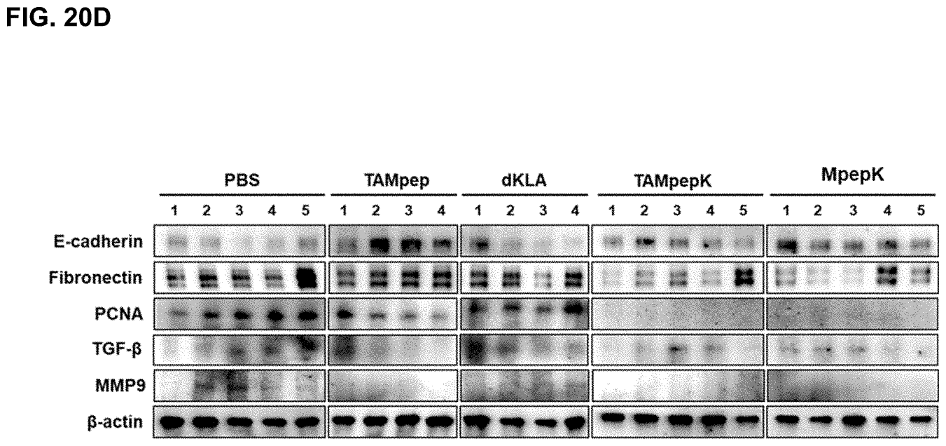

To determine anti-cancer effect of TAMpepK and MpepK in tumor growth and EMT of prostate cancer model, tumor tissues were measured expression of PCNA as proliferative marker and E-cadherin, vimentin, fibronectin, TGF-β and MMP9 as EMT (epithelial-mesenchymal transition) markers. Expression of PCNA was reduced in TAMpepK and MpepK groups (FIGS. 20C and 20D). In EMT marker, E-cadherin known as epithelial cell marker was increased in TAMpepK and MpepK groups (FIGS. 20A and 20D), while vimentin and fibronectin known as mesenchymal marker were reduced in TAMpepK and MpepK groups (FIGS. 20B and 20D). Moreover, expression of TGF-β and MMP9 related with EMT was also reduced in TAMpepK and MpepK groups (FIG. 20D). Thus, these findings suggest that TAMpepK and MpepK have anti-cancer effect by inhibiting tumor growth and metastasis targeting M2-like TAMs in prostate cancer.

2-21. Anti-Cancer Effect of TAMpepK and MpepK in Colon Cancer Model

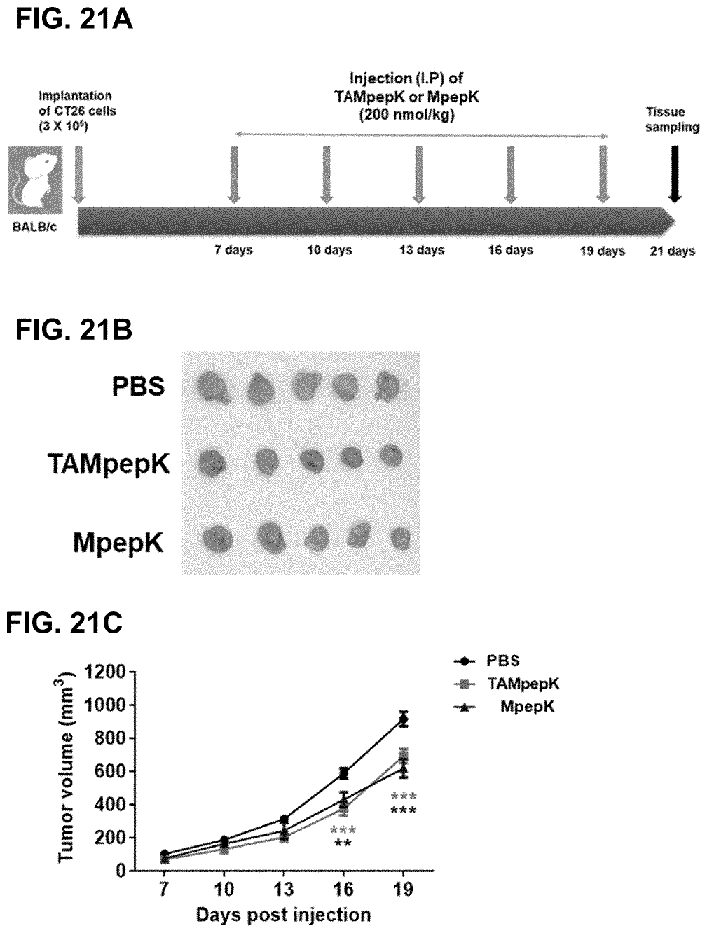

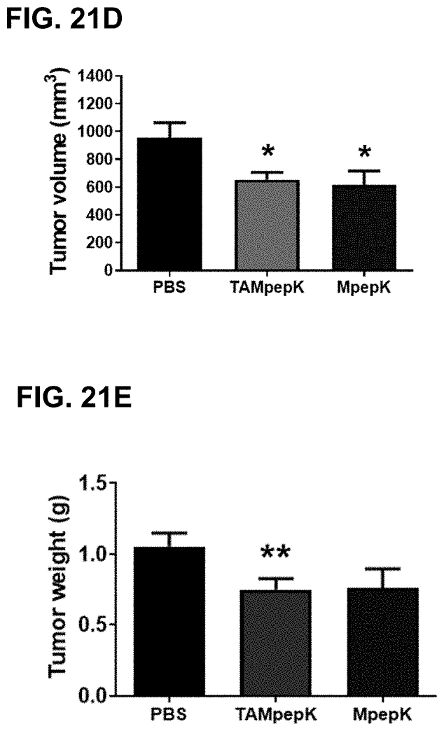

To determine anti-cancer effect of TAMpepK and MpepK in tumor growth of colon cancer model, tumor tissues were measured for volume and weight. Mice treated with TAMpepK and MpepK showed significantly reduced tumor volume and weight compared to the PBS group, whereas the tumor weight was not significantly changed in MpepK (FIGS. 21A-21E).

2-22. Effect of MpepK in Mouse Model for Lung Fibrosis

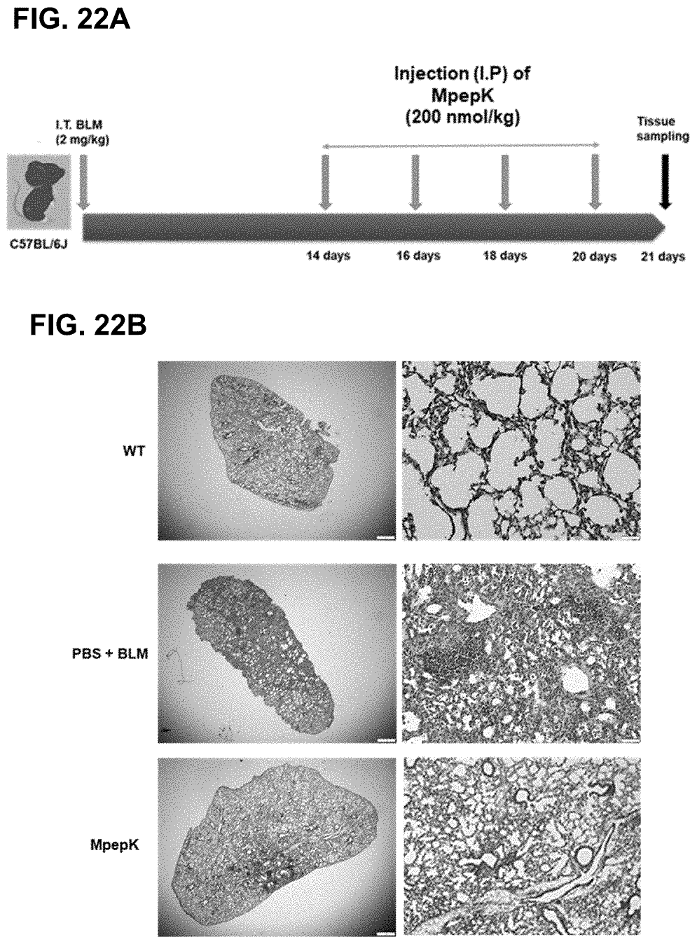

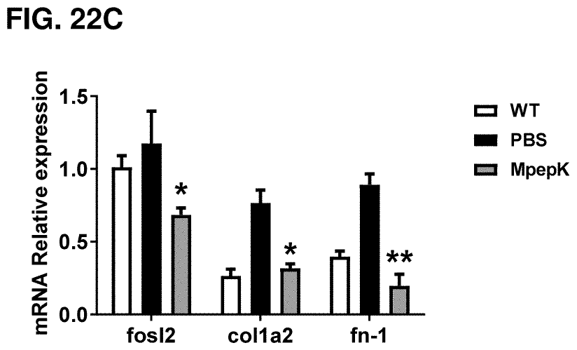

To determine whether MpepK has therapeutic effect for inhibition of lung fibrosis, mouse model of lung fibrosis was established by intratracheally administrating bleomycin. Lung fibrosis induced by bleomycin was decreased by MpepK (FIG. 22B). Additionally, gene expression related to fibrosis such as fos12, collagen type 1 and fibronectin 1 was significantly reduced in MpepK compared to PBS (FIG. 22C).

2-23. Effect of TAMpepK and MpepK in Mouse Model for Breast Cancer

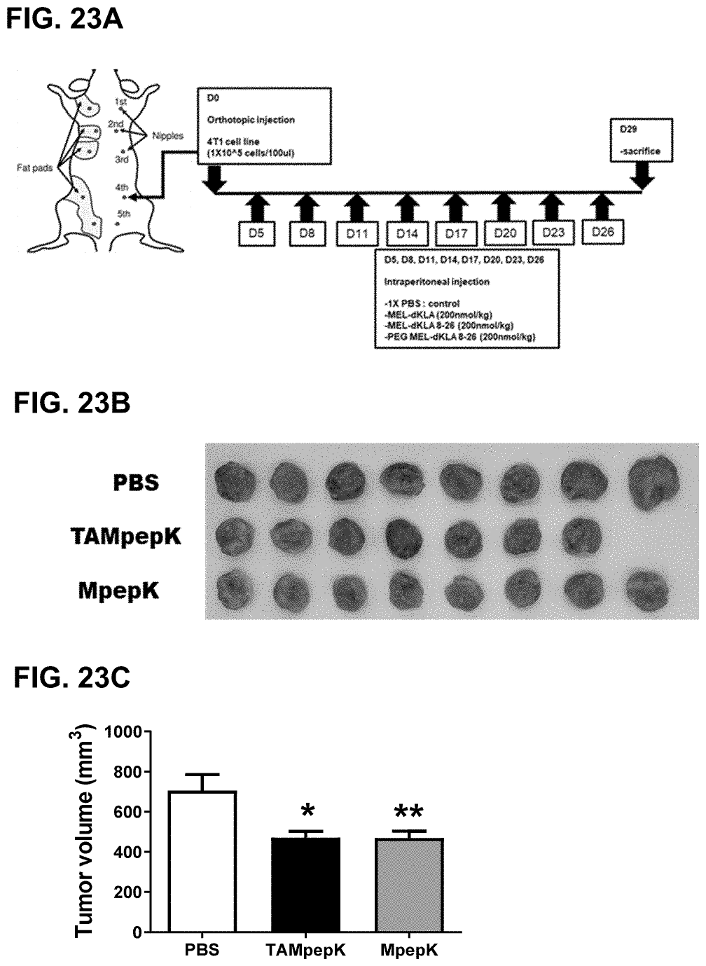

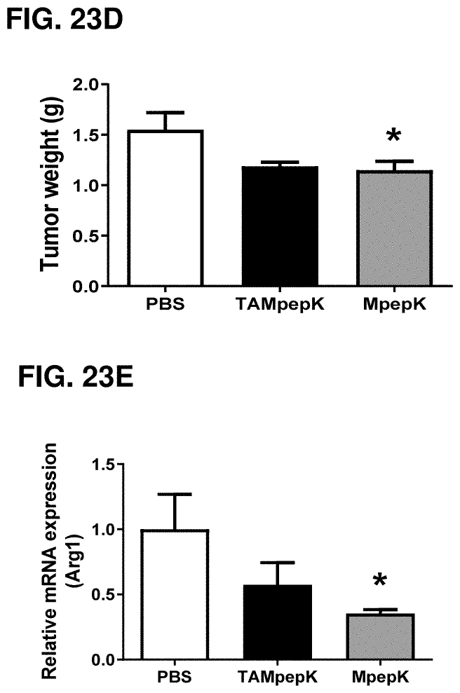

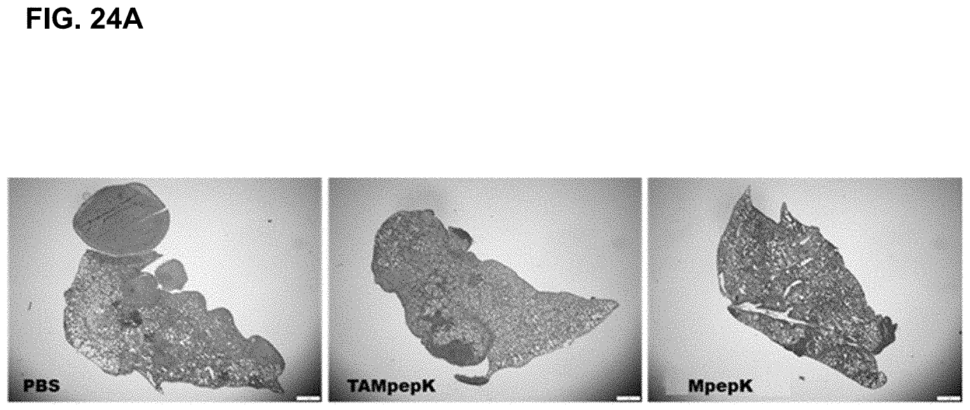

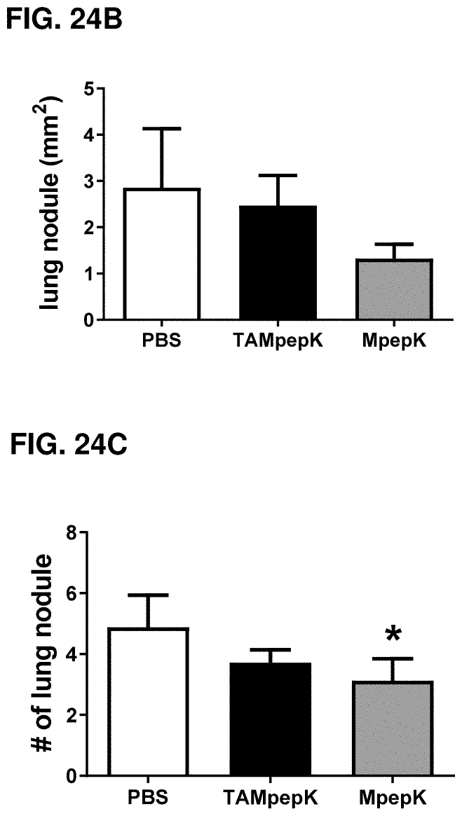

To determine the anti-cancer effect of TAMpepK and MpepK in breast cancer, the 4^{th} mammary orthotopic mouse model of breast cancer was established. TAMpepK and MpepK showed decreased tumor volume and weight compared to the PBS group (FIGS. 23B-23D). Moreover, gene expression of arginase 1 known as M2 macrophage marker was significantly reduced in MpepK compared to PBS (FIG. 23E). In addition, lung metastasis was decreased in the MpepK group compared to the PBS group (FIGS. 24A-24C).

### Example 3

### Materials and Methods

3-1. Peptide synthesis.

The following peptides were synthesized according to the method described above in Example 1:
TAMpep: full length melittin (SEQ ID NO:1);
Mpep: full length melittin with the first 7 amino acids removed (SEQ ID NO:2);
A12: the fifth G (glycine) of Mpep is substituted with alanine (SEQ ID NO:16);
A14: the seventh P (proline) of Mpep is substituted with alanine (SEQ ID NO:18);
A17: the tenth I (isoleucine) of Mpep is substituted with alanine (SEQ ID NO:20);
A18: the eleventh S (serine) of Mpep is substituted with alanine (SEQ ID NO:21);
A22: the fifteenth R (arginine) of Mpep is substituted with alanine (SEQ ID NO:25);
A25: the eighteenth Q (glutamine) of Mpep is substituted with alanine (SEQ ID NO:28);
A26: the nineteenth Q (glutamine) of Mpep is substituted with alanine (SEQ ID NO:29);
TAMpepK: a full-length melittin peptide (SEQ ID NO:1) attached to a linker (GGGGS);
(SEQ ID NO:36), which is attached to d-Lys-d-Leu-d-Ala-d-Lys-d-Leu-d-Ala-d-Lys-d-Lys-d-Leu-d-Ala-d-Lys-d-Leu-d-Ala-d-Lys (dKLA) (SEQ ID NO:47);
MpepK: Mpep (SEQ ID NO:2) attached to a linker (GGGS) (SEQ ID NO:36), which is attached to d-Lys-d-Leu-d-Ala-d-Lys-d-Leu-d-Ala-d-Lys-d-Lys-d-Leu-d-Ala-d-Lys-d-Leu-d-Ala-d-Lys (dKLA) (SEQ ID NO:47);
A12K: A12 (SEQ ID NO:16) attached to a linker (GGGS) (SEQ ID NO:36), which is attached to d-Lys-d-Leu-d-Ala-d-Lys-d-Leu-d-Ala-d-Lys-d-Lys-d-Leu-d-Ala-d-Lys-d-Leu-d-Ala-d-Lys (dKLA) (SEQ ID NO:47);
A14K: A13 (SEQ ID NO:18) attached to a linker (GGGS) (SEQ ID NO:36), which is attached to d-Lys-d-Leu-d-Ala-d-Lys-d-Leu-d-Ala-d-Lys-d-Lys-d-Leu-d-Ala-d-Lys-d-Leu-d-Ala-d-Lys (dKLA) (SEQ ID NO:47);
A17K: A17 (SEQ ID NO:20) attached to a linker (GGGS) (SEQ ID NO:36), which is attached to d-Lys-d-Leu-d-Ala-d-Lys-d-Leu-d-Ala-d-Lys-d-Lys-d-Leu-d-Ala-d-Lys-d-Leu-d-Ala-d-Lys (dKLA) (SEQ ID NO:47);
A18K: A18 (SEQ ID NO:21) attached to a linker (GGGS) (SEQ ID NO:36), which is attached to d-Lys-d-Leu-d-Ala-d-Lys-d-Leu-d-Ala-d-Lys-d-Lys-d-Leu-d-Ala-d-Lys-d-Leu-d-Ala-d-Lys (dKLA) (SEQ ID NO:47);
A22K: A22 (SEQ ID NO:25) attached to a linker (GGGS) (SEQ ID NO:36), which is attached to d-Lys-d-Leu-d-Ala-d-Lys-d-Leu-d-Ala-d-Lys-d-Lys-d-Leu-d-Ala-d-Lys-d-Leu-d-Ala-d-Lys (dKLA) (SEQ ID NO:47);
A25K: A25 (SEQ ID NO:28) attached to a linker (GGGS) (SEQ ID NO:36), which is attached to d-Lys-d-Leu-d-Ala-d-Lys-d-Leu-d-Ala-d-Lys-d-Lys-d-Leu-d-Ala-d-Lys-d-Leu-d-Ala-d-Lys (dKLA) (SEQ ID NO:47); and
A26K: A26 (SEQ ID NO:29) attached to a linker (GGGS) (SEQ ID NO:36), which is attached to d-Lys-d-Leu-d-Ala-d-Lys-d-Leu-d-Ala-d-Lys-d-Lys-d-Leu-d-Ala-d-Lys-d-Leu-d-Ala-d-Lys (dKLA) (SEQ ID NO:47).

3-2. Macrophage Differentiation.

THP-1 monocytes were differentiated into macrophages (M0) by 24 h incubation with 100 nM phorbol 12-myristate 13-acetate (PMA, Sigma) followed by 24 h incubation in RPMI medium (Invitrogen). Macrophages were polarized in M1 macrophages (M1) by incubation with 20 ng/ml of IFN-γ (Prospec) and 100ng/m1 of LPS (Sigma). Macrophage M2 polarization(M2) was obtained by incubation with 20 ng/ml of interleukin (IL) 4 (Prospec) and 20 ng/ml of interleukin 13 (Pro spec).

3-3. Cell Viability Tests.

Polarized cells were treated with 1.5 μM MpepK, A12K, A14K, A17K, A18K, A22K, A25K or A26K peptides for 1 hour and further incubated in RPMI1640 growth medium for 24 hours. Cell viability was analyzed using the CCK-8 assay: CCK-8 reagent (Enzo Life Sciences) was added to each well; incubation was continued for 2 hours, and absorbance was measured at 450 nm with a microplate reader (Molecular Devices).

3-4. Cytotoxicity of A26K in In Vitro Sepsis Model, LPS-Stimulated M1 (LPS-M1) Macrophages

THP-1 cells (1×10⁴ cells/well) were differentiated into macrophages with 100 nM PMA (MO) for 24 h and polarized into classical M1 macrophages by treatment of IFN-γ (20 ng/ml) and LPS (100 ng/ml) and LPS-stimulated macrophages (LPS-M1) were induced by LPS (1 m/ml) treatment for 24h. Cells were treated with 1.5 μM of A26K for 1 hour and further incubated in RPMI1640 growth medium for 24 hours. Cell viability was analyzed using the CCK-8 assay. CCK-8 reagent was added to each well and incubated for 1.5-2 hours. Absorbance was measured at 450 nm with a microplate reader.

3-5. Effects of A26K Treatment in LPS-M1 Macrophages

THP-1 cells (2×10⁵ cells/well) were differentiated into macrophages with 100 nM PMA (MO) for 24h and polarized into classical M1 macrophages by treatment of IFN-γ (20 ng/ml) and LPS (100 ng/ml) and LPS-M1 macrophages were induced by LPS (1 m/ml) treatment for 2 h. Polarized cells were treated with 1.5 μM of A26K for 1 hour and further incubated in RPMI1640 growth medium for 24 hours. Expression levels of pro-inflammatory genes (IL-8, TNF-α, NF-kB, IL-1(3 and CXCL10) were quantified by real-time quantitative PCR.

3-6. Lung Fibrosis In Vitro Model-Cells

THP-1 cells were purchased from the American Type Culture Collection (ATCC) and cultured according to their specific indications, using an RPMI 1640 medium supplemented with non-heat-treated 10% fetal bovine serum (FBS; WelGENE), 2 mM L-glutamine, 0.05 mM β-mercaptoethanol, 10 mM HEPES, 4500 mg/L glucose, 100 U/ml penicillin and 100 μg/ml streptomycin (Gibco). Human alveolar cell, A549 cells, obtained from the American Type Culture Collection (ATCC), were cultured in RPMI 1640 medium containing 2.05 mM L-glutamine, 2 g/liter sodium bicarbonate and 2 g/liter glucose (WelGENE) together with 10% FBS (WelGENE), 100 U/ml penicillin and 100 μg/m1 streptomycin (Gibco). Cells were cultured at 37° C. in a 5% CO2 humidified incubator to reach 80% of confluence.

3-7. Lung Fibrosis In Vitro Model—Macrophage Differentiation

THP-1 cells are differentiated into macrophages by 24 h incubation with 100 nM phorbol 12-myristate 13-acetate (PMA, Sigma) followed by 24 h incubation in RPMI medium (Invtrogen). Macrophage M2 polarization (M2) was obtained by incubation with 20 ng/ml of interleukin (IL)-4 (Prospec) and 20 ng/ml of interleukin 13 (Prospec).

3-8. Lung Fibrosis In Vitro Model—Treatment of the Cultured Cells and Coculture

A non-contact co-culture system of THP-1 and A549 cells was established using a

Transwell suspension culture chamber with polyethylene terephthalate film combined with a 6-pore plate (Corning 3450; Corning, Inc., Corning, NY, USA). A549 cells with a seeding density of 1×10⁵ cells/ml in six-well plates were cultured in medium containing TGF-β (5 ng/ml) for 48h to induce EMT or FMT in vitro. MpepK , A17K or A22K were synchronously used to observe the intervention effect on the treated cells. THP-1 cells seeded at a density of 1×10⁵ cells/ml were exposed to 20 ng/ml of IL-4 and 20 ng/ml of IL-13 for 48 h to induce M2-. Some of the cells were also treated with 1.5 μM MpepK, A17K and A22K. To establish the coculture with M2-like macrophages, we transferred the cell culture inserts containing IL-4 and IL-13 pretreated macrophages to the plates that had been seeded with A549 cells (5×10⁴ cells/ml) for culturing 24 h. After 48 h of coculture, the cells at the bottom of the plates were harvested for further experiments.

3-9. Lung Fibrosis In Vitro Model—Real-Time Quantitative PCR

Total RNA was extracted using Easy-Blue reagent. Concentrations of RNA were determined and quantified by measuring absorbance at 260 and 280 nm with a spectrophotometer. Complementary DNA (cDNA) was synthesized from total RNA using a Maxime RT PreMix kit (iNtRON). Real-time PCR analysis was performed with SYBR Green Master Mix. PCR conditions were forty-five cycles at 95° C. for 5 min, followed by 95° C. for 10 sec, 60° C. for 10 sec and 72° C. for 10 sec. mRNA expression were quantified in triplicate. Data were measured with CFX Software (Bio-Rad). GAPDH were used as internal controls.

3-10. Anti-Cancer Effect of MpepK in Mouse Model of Hepatocellular Carcinoma

C57BL/6 (B6) wild-type mice purchased from DBL. For the subcutaneous tumor model of hepatocellular carcinoma, Hepa1-6 cells were mixed with Matrigel matrix (Corning) and inoculated subcutaneously into the right flank (4×10⁵ cells/mouse) of the mice. MpepK peptide (100, 200 and 400 nmol/kg) were injected intraperitoneally every 3 days, beginning at day 12 after tumor inoculation and tumor volume was measured by electronic caliper. All animals were maintained in a specific pathogen-free environment on a 12-h light/dark cycle with free access to food and water. After the experiments were terminated, all mice were euthanized using isoflurane and cervical dislocation.

### Example 4

### Results

4-1. Cytotoxicity of Polypeptides Selective for M2-type, M1-type, and/or M0 type Macrophages.

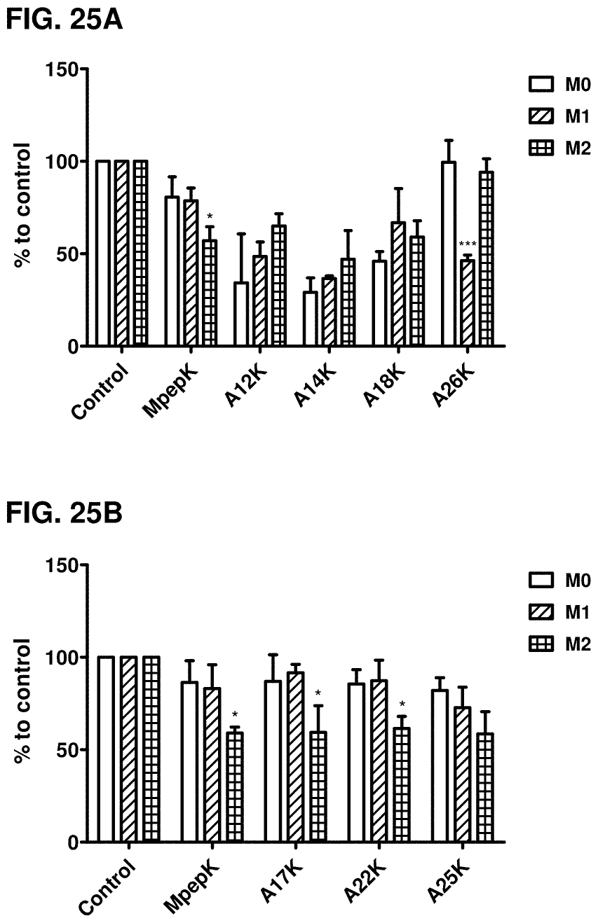

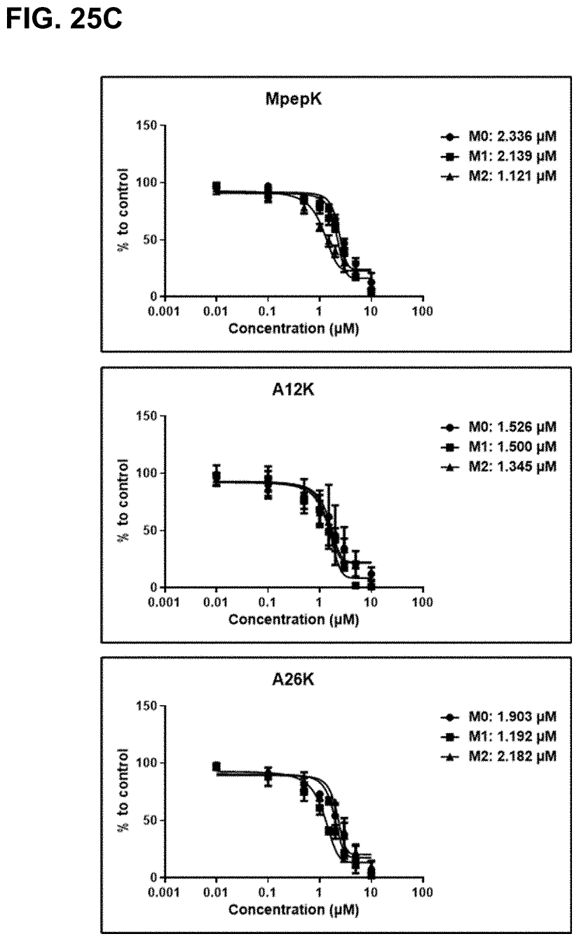

Among the alanine substituted Mpep, some peptides showed relatively increased affinity in M1 macrophages compared to M2 macrophages or relatively increased affinity in M0 macrophages compared to M1 or M2 macrophages (FIG. 7A). To assess whether the increased affinity affect to selective cytotoxicity in M0 or M1 or M2 macrophages, 1.5 μM of MpepK, A12K, A14K, A17K, A18K, A22K, A25K or A26K peptides were treated in M0, M1 and M2 macrophages and measured cell viability using the CCK-8 assay. As a result, A26K peptide showed great significant selective cytotoxic effect in only M1 macrophages compared to control M1 macrophage (***p<0.001, compared to control M1 macrophage) while A12K, A14K and A18K peptides did not show selective cytotoxicity in M0, M1 and M2 macrophages (FIG. 25A). A17K, A22K and A25K showed significant cytotoxic effects similar with MpepK in M2 macrophages (*p<0.05, compared to control M2 macrophage), but not in M0 and M1 macrophages (FIG. 25B). When M0, M1 and M2 macrophages were treated with increasing concentration of A26K (0.01-10 μM), A26K peptide showed 1.192 μM at IC50 in M1 macrophages (FIG. 25C).

4-2. Cytotoxicity and Effects of A26K in In Vitro Sepsis Model, LPS-Stimulated M1 (LPS-M1) Macrophages.

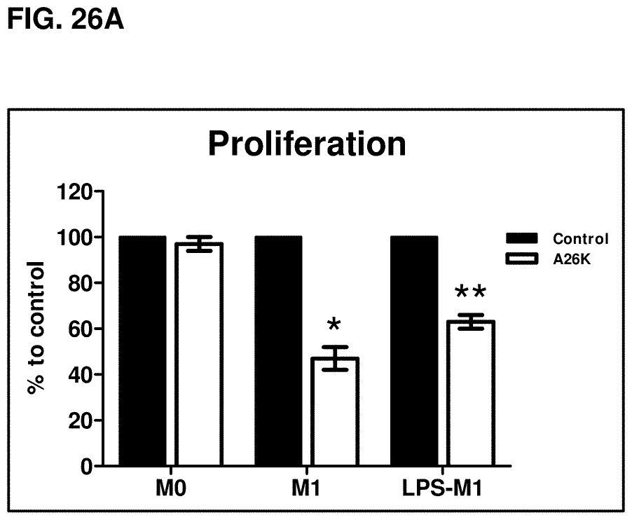

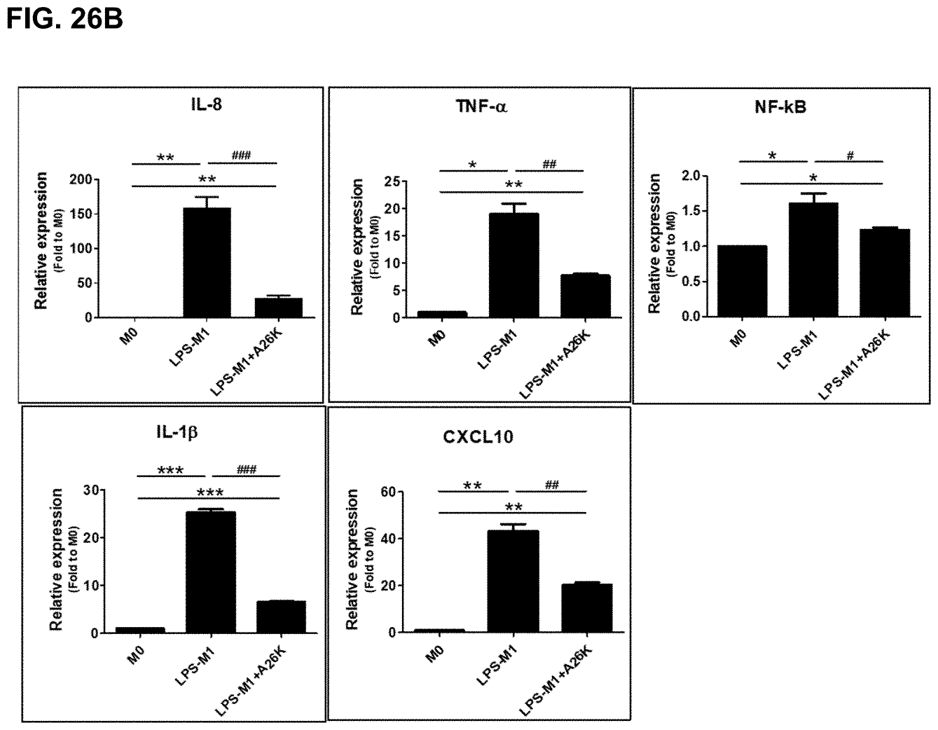

Sepsis is the systemic inflammatory response to an infection of microbial pathogens. LPS is the part of the outer membranes of gram-negative bacteria and induces multiple inflammatory responses in monocytes and macrophages in vivo and in vitro. Therefore, LPS-mediated inflammatory response is a major inflammation source from exposure to gram-negative bacterial infection and is closely related to sepsis. To examine the cytotoxicity of A26K in LPS-M1 macrophages, M0, M1, and LPS-M1 macrophages were treated with 1.5 μM of A26K. As a results, A26K showed significant cytotoxic effects in LPS-M1 macrophages (37% inhibition, *p<0.05, compared to control) and M1 macrophages (53% inhibition, *p<0.05, compared to control) (FIG. 26A). To further examine the expression levels of pro-inflammatory genes, M0, M1, and LPS-M1 macrophages were treated with 1.5 μM of A26K for 1 h. LPS (1 m/ml) stimulation significantly increased the expression of IL8, TNF-α, IL-1β, NF-kB and CXCL10, compared to M0 macrophages (*p<0.05 or **p<0.01 or ***p<0.001, compared to M0 macrophages, FIG. 26B). However, A26K treatment significantly inhibited the enhanced expression levels of IL8, TNF-α, IL-1β, NF-kB and CXCL10 by LPS stimulation (#p<0.05 or ##p<0.01 or ###p<0.001, compared to LPS-M1 macrophages, FIG. 26B). These results indicated that A26K treatment significantly suppressed the activation of in vitro sepsis model, M1 macrophages induced by LPS. Therefore, A26K treatment can be controlling the early excessive inflammatory response by inhibition of M1 macrophages and would be an important and effective treatment for sepsis.

4-3. Effects of A17K or A22K in In Vitro Lung Fibrosis Model, TGF-β1-Induced A549 Cells Cocultured with IL-4 and IL-13 Induced THP-1 Macrophages.

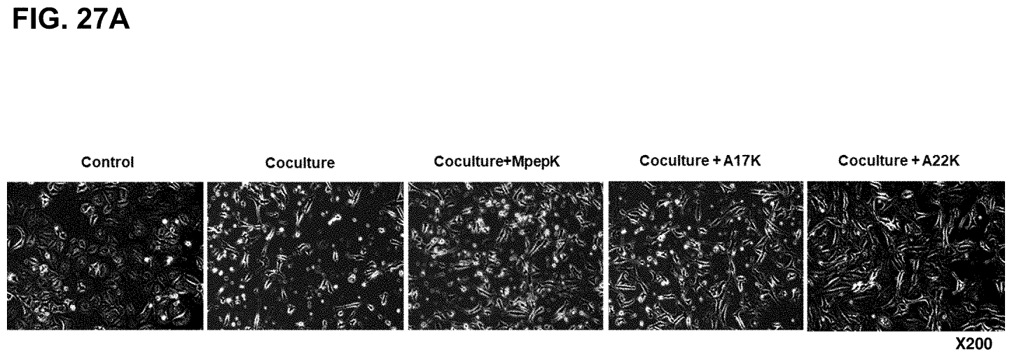

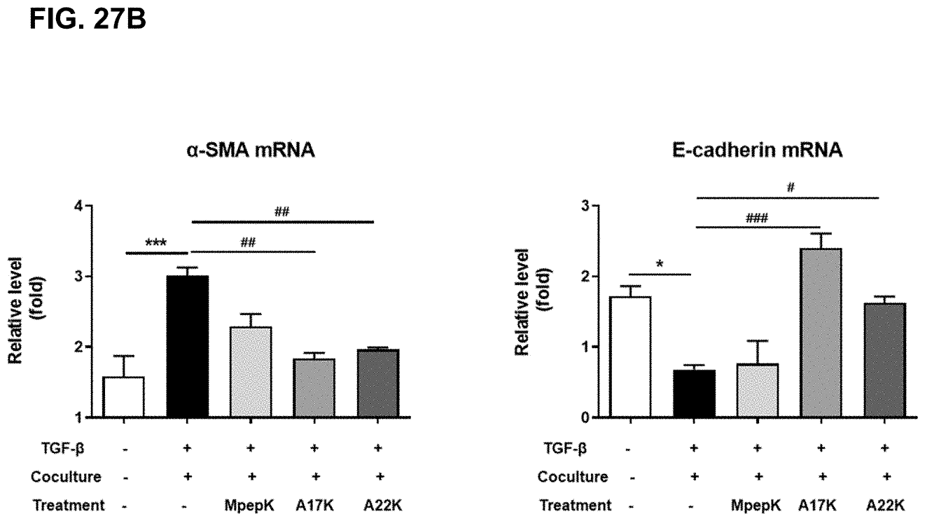

To investigate the effect of A17K or A22K treatment on epithelial-mesenchymal transition (EMT) and fibroblast to myofibroblast transition (FMT) responses, we cultured A549 (commonly used as a model of human alveolar type II pulmonary epithelium) in the presence of TGF-β1-induced acquisition of mesenchymal characteristics or fibrotic markers in A549 cells. The morphological changes were imaged using phase contrast microscopy (shown at 200× magnification). We induced EMT in A549 cells, the most popular cell lines of human alveolar epithelial type II cells, with treatment of TGF-β1 for 48 h. Using a cell coculture system, TGF-β1-induced A549 cells were cocultured with IL-4 and IL-13 induced THP-1 macrophages. It was clearly detected morphological alteration in A549 from oval epithelial cells to spindle shaped fibroblast-like cells. A17K or A22K intervention markedly blocked the spindle-like mesenchymal morphology phenotype of EMT in A549 cells stimulated by cocultured with IL-4 and IL-13 induced macrophages (FIG. 27A). A17K or A22K treatment significantly enhanced the expression of E-cadherin, EMT inhibition marker and reduced the expression of α-SMA, FMT enhancement marker in A549 cells compared with those of M2 macrophage alone (#p<0.05 or ##p<0.01 or ###p<0.001, compared to M2 macrophages). However, no significant inhibitory effect of MpepK was observed on EMT and FMT of these epithelial cells when cocultured with M2 polarization of THP-1 (FIGS. 27A and 27B). Those results suggest A17K or A22K show better inhibitions of lung fibrosis than MpepK and would be greater therapeutics for lung fibrosis.

4-4. Anti-Cancer Effect of MpepK in Mouse Model of Hepatocellular Carcinoma.

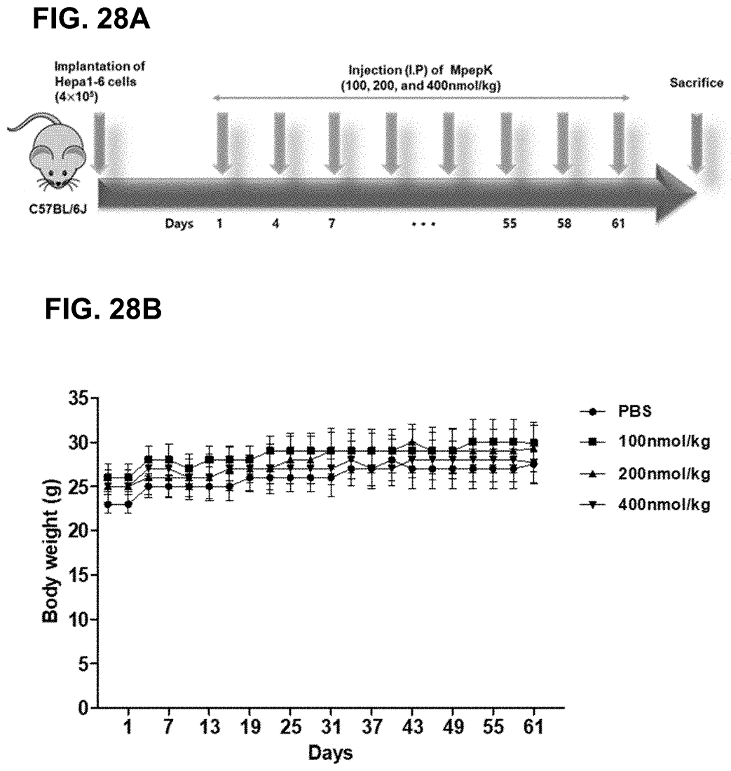

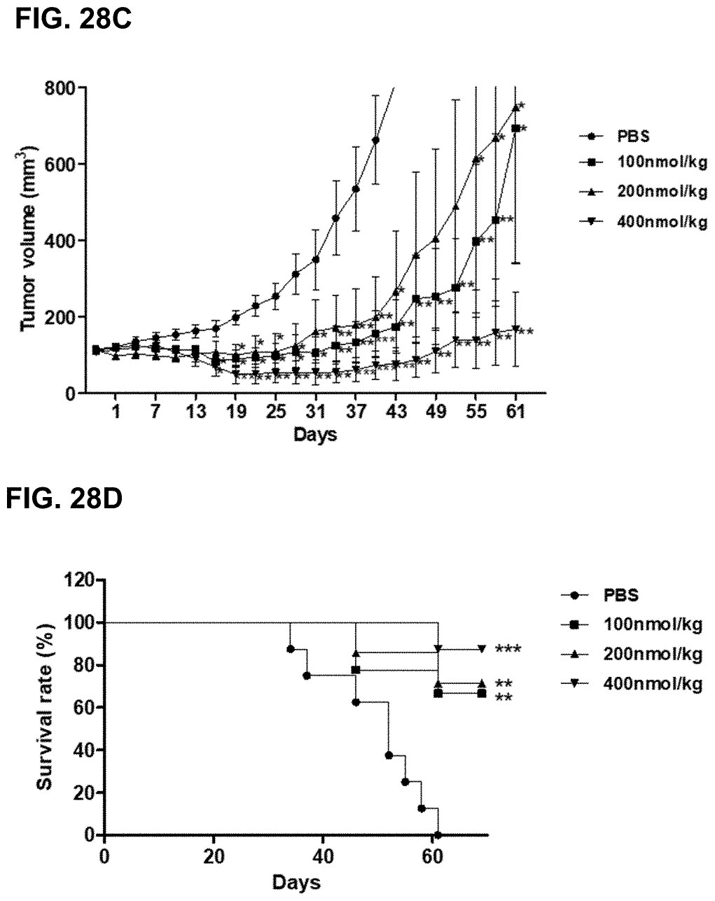

To assess anti-cancer effect of MpepK in vivo, mouse hepal-6 cells were injected subcutaneously in right flank of C57BL/6J mice. 12 days after cell inoculation, MpepK was injected intraperitoneally every 3 days (FIG. 28A). As a result, there was no significant difference in body weight change between the groups (FIG. 28B). On the other hand, mice treated with MpepK of all doses (100, 200 and 400 nmol/kg) showed significantly reduced tumor volume compared with PBS group, and survival rate was significantly extended in MpepK groups (100, 200 and 400 nmol/kg) compared to PBS group (*p<0.05 or **p<0.01 or ***p<0.001, compared to PBS group, FIGS. 28C and 28D).

The foregoing description of the specific embodiments will so fully reveal the general nature of the disclosure that others can, by applying knowledge within the skill of the art, readily modify and/or adapt for various applications, without departing from the general concept of the disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be herein incorporated by reference.

1. A polypeptide comprising the amino acid sequence of X1-X2-Thr-X4-Gly-Leu-X7-Ala-Leu-Ile-X11-Trp-Ile-X14-Arg-Lys-Arg-X18-X19 (SEQ ID NO:3), wherein X1 is an amino acid other than valine, X2 is an amino acid other than leucine, X4 is an amino acid other than threonine, X7 is an amino acid other than proline, X11 is an amino acid other than serine, X14 is an amino acid other than lysine, X18 is an amino acid other than glutamine, and/or X19 is an amino acid other than glutamine.

2. The polypeptide of claim 1, wherein the X1 is alanine (SEQ ID NO:4).

3. The polypeptide of claim 1, wherein the X2 is alanine (SEQ ID NO:5).

4. The polypeptide of claim 1, wherein the X4 is alanine (SEQ ID NO:6).

5. The polypeptide of claim 1, wherein the X7 is alanine (SEQ ID NO:7).

6. The polypeptide of claim 1, wherein the X11 is alanine (SEQ ID NO:8).

7. The polypeptide of claim 1, wherein the X14 is alanine (SEQ ID NO:9).

8. The polypeptide of claim 1, wherein the X18 is alanine (SEQ ID NO:10).

9. The polypeptide of claim 1, wherein the X19 is alanine (SEQ ID NO:11).

10. A conjugate comprising the polypeptide of claim 1 and a second therapeutic drug.

11. The conjugate of claim 10, wherein the second therapeutic drug is selected from the group consisting of KLA, alpha-defensin-1, BMAP-28, brevenin-2R, buforin IIb, cecropin A-magainin 2 (CA-MA-2), cecropin A, cecropin B, chrysophsin-1, D-K6L9, gomesin, lactoferricin B, LLL27, LTX-315, magainin 2, magainin IIbombesin conjugate (MG2B), pardaxin, doxorubicin, methotrexate, entinostat, cladribine, pralatrexate, lorlatinib, maytansine DM1, maytansine DM3, maytansine DM4, and combinations thereof.

12. The conjugate of claim 10, further comprising a linker that links the polypeptide to the second therapeutic drug.

13. The conjugate of claim 12, wherein both ends of the linker comprise a functional group selected from the group consisting of carbodiimide, N-hydroxysuccinimide ester (NHS ester), imidoester, pentafluoropheny ester, hydroxymethyl phosphine, maleimide, haloacetyl, pyridyldi sulfide, thiosulfonate, vinyl sulfone, EDC (1-ethyl-3 -(3-dimethylaminopropyl)carbodiimide), DCC (N,N′-dicyclohexylcarbodiimide), SATA (succinimidyl acetylthioacetate), sulfo-SMCC (sulfosuccinimidyl-4-(NDmaleimidomethyl) cyclohexane-1-carboxylate), DMA (dimethyl adipimidate 2HCl), DMP (dimethylpimelimidate2HCl), DMS (dimethyl Suberimidate2HCl), DTBP (dimethyl 3,3′-dithiobispropionimidate 2HCl), sulfo-SIAB (sulfosuccinimidyl(4-iodoacetyl)aminobenzoate), STAB (succinimidyl(4-iodoacetyl)aminobenzoate), SBAP (succinimidyl 3 -(bromoacetamido) propionate), SIA (succinimidyl iodoacetate), SM(PEG)n (succinimidyl-([Nmaleimidopropionamido]-ethyleneglycol ester, wherein n=2, 4, 6, 8, 12 or 24), SMCC(succinimidyl-4-(N-Dmaleimidomethyl)cyclohexane-1-carboxylate), LC SMCC (succinimidyl 4-(N-maleimidomethyl)cyclohexane-1-carboxy-(6-amidocaproate)), sulfo-EMCS (N-cester),EMCS (N-εsulfo-GMBS(N-γester), GMBS (N-γester), sulfo-KMUS (N-κester), sulfo-MBS (mmaleimidobenzoyl-Nhydroxysulfosuccinimide ester), MBS (m-maleimidobenzoyl-Nhydroxysuccinimide ester), sulfo-SMPB (sulfosuccinimidyl 4-(p-maleimidophenyl)butyrate), SMPB (succinimidyl 4-(pmaleimidophenyl)butyrate), AMAS (N-α-maleimidoacetoxysuccinimide ester), BMPS (N-β-maleimidopropyloxysuccinimide ester), SMPH (succinimidyl 6-[(β-maleimidopropionamido)hexanoate]), PEG12-SPDP (2-pyridyldithioltetraoxaoctatriacontane-N-hydroxysuccinimide), PEG4-SPDP, sulfo-LCSPDP (sulfosuccinimidyl 6-[3′-(2-pyridyldithio)propionamido]hexanoate), SPDP (succinimidyl 3-(2-pyridyldithio)propionate), LC-SPDP (succinimidyl 6-[3′-(2-pyridyldithio) propionamido]hexanoate), SMPT (4-succinimidyloxycarbonyl-alpha-methylalpha(2-pyridyldithio)toluene), DSS (di succinimidyl suberate), BS (PEG)5 (bis(succinimidyl) penta(ethylene glycol)), B S(PEG)9 (bis(succinimi dyl) nona(ethylene glycol)), BS3 (bis[sulfosuccinimidyl]suberate), B SOCOES (bis[2-(succinimidooxycarbonyloxy) ethyl]sulfone), PDPH (3-(2-pyridyldithio)propionyl hydrazide), DSG (di succinimidyl glutarate), DSP (dithiobis[succinimidyl propionate]), BM(PEG)n (1,8-bismaleimido-ethyleneglycol, n=2 or 3), BMB (1,4-bismaleimidobutane), BMDB (1,4-bismaleimidyl-2,3-dihydroxybutane), BMH (bismaleimidohexane), BMOE (bismaleimidoethane), DTME (dithiobismaleimidoethane), TMEA (tris(2-maleimidoethyl)amine), DSS (disuccinimidyl suberate), DST (disuccinimidyl tartarate), DTSSP (3,3′-dithiobis[sulfosuccinimidylpropionate]), EGS (ethylene glycol bis[succinimidylsuccinate]), sulfo-EGS (ethylene glycol bis[sulfosuccinimidylsuccinate]), TSAT (tris-succinimidyl aminotriacetate), DFDNB (1,5-difluoro-2,4-dinitrobenzene), and combinations thereof.

14. A pharmaceutical composition comprising the polypeptide of claim 1 and a pharmaceutically acceptable carrier.

15.-17. (canceled)

18. A method of decreasing M2-type macrophages or treating an M2-type macrophage-mediated disease in a subject in need thereof, comprising administering a polypeptide of claim 1 to the subject.

19. The method of claim 18, wherein the polypeptide decreases M2-tpe macrophages compared to a polypeptide having the amino acid sequence of SEQ ID NO:2.

20. The method of claim 18, wherein the disease is a cancer.

21. The method of claim 20, wherein the cancer is melanoma, prostate cancer, lung cancer, breast cancer, colon cancer, pancreatic cancer, or other solid tumors having M2-type tumor-associated macrophages in a cancer microenvironment.

22. The method of claim 18, wherein the disease is a fibrosis-related disease, end-stage liver disease, kidney disease, idiopathic pulmonary fibrosis (IPF), heart failure, many chronic autoimmune diseases, including scleroderma, rheumatoid arthritis, Crohn's disease, ulcerative colitis, myelofibrosis and systemic lupus erythematosus, tumor invasion and metastasis, chronic graft rejection and the pathogenesis of many progressive myopathies, liver cirrhosis and fibrosis, benign prostatic hyperplasia, or prostatitis.

23. A method of decreasing M1-type macrophages or treating an M1-type macrophage-mediated disease in a subject in need thereof, comprising the polypeptide of claim 1 to the subject.

24. The method of claim 23, wherein the polypeptide decreases M1-type macrophages compared to a polypeptide having the amino acid sequence of SEQ ID NO:2.

25. The method claim 23, wherein the disease is a chronic inflammatory disease including septic shock, multiple organ dysfunction syndrome (MODS), atopic dermatitis, rheumatoid arthritis, or autoimmune disorders.

26. A method of decreasing M0-type macrophages or treating an M0-type macrophage-mediated disease in a subject in need thereof, comprising administering the polypeptide of claim 1 to the subject.

27. The method of claim 26, wherein the polypeptide decreases M0-type macrophages compared to a polypeptide having the amino acid sequence of SEQ ID NO:2.

28. The method of claim 18, wherein the polypeptide is linked to a second therapeutic drug.

29. (canceled)

30. The method of claim 18, wherein the polypeptide is linked to the second therapeutic drug by a linker.

31.-33. (canceled)

34. The method of claim 20, wherein the cancer is hepatocellular cancer.


SEQUENCE LISTING (55 sequences)

SEQ ID NO: 1 - protein, 26 aa, Artificial Sequence
  MEL
GIGAVLKVLTTGLPALISWIKRKRQQ

SEQ ID NO: 2 - protein, 19 aa, Artificial Sequence
  MEL826 or Mpep
VLTTGLPALISWIKRKRQQ

SEQ ID NO: 3 - protein, 19 aa, Artificial Sequence
  Mpep
  MISC_FEATURE (1)..(1): X is an amino acid other than valine
  MISC_FEATURE (2)..(2): X is an amino acid other than leucine
  MISC_FEATURE (4)..(4): X is an amino acid other than threonine
  MISC_FEATURE (7)..(7): X is an amino acid other than proline
  MISC_FEATURE (11)..(11): X is an amino acid other than serine
  MISC_FEATURE (14)..(14): X is an amino acid other than lysine
  MISC_FEATURE (18)..(18): X is an amino acid other than glutamine
  MISC_FEATURE (19)..(19): X is an amino acid other than glutamine
XXTXGLXALIXWIXRKRXX

SEQ ID NO: 4 - protein, 19 aa, Artificial Sequence
  Mpep
  MISC_FEATURE (2)..(2): X is an amino acid other than leucine
  MISC_FEATURE (4)..(4): X is an amino acid other than threonine
  MISC_FEATURE (7)..(7): X is an amino acid other than proline
  MISC_FEATURE (11)..(11): X is an amino acid other than serine
  MISC_FEATURE (14)..(14): X is an amino acid other than lysine
  MISC_FEATURE (18)..(18): X is an amino acid other than glutamine
  MISC_FEATURE (19)..(19): X is an amino acid other than glutamine
AXTXGLXALIXWIXRKRXX

SEQ ID NO: 5 - protein, 19 aa, Artificial Sequence
  Mpep
  MISC_FEATURE (1)..(1): X is an amino acid other than valine
  MISC_FEATURE (4)..(4): X is an amino acid other than threonine
  MISC_FEATURE (7)..(7): X is an amino acid other than proline
  MISC_FEATURE (11)..(11): X is an amino acid other than serine
  MISC_FEATURE (14)..(14): X is an amino acid other than lysine
  MISC_FEATURE (18)..(18): X is an amino acid other than glutamine
  MISC_FEATURE (19)..(19): X is an amino acid other than glutamine
XATXGLXALIXWIXRKRXX

SEQ ID NO: 6 - protein, 19 aa, Artificial Sequence
  Mpep
  MISC_FEATURE (1)..(1): X is an amino acid other than valine
  MISC_FEATURE (2)..(2): X is an amino acid other than leucine
  MISC_FEATURE (7)..(7): X is an amino acid other than proline
  MISC_FEATURE (11)..(11): X is an amino acid other than serine
  MISC_FEATURE (14)..(14): X is an amino acid other than lysine
  MISC_FEATURE (18)..(18): X is an amino acid other than glutamine
  MISC_FEATURE (19)..(19): X is an amino acid other than glutamine
XXTAGLXALIXWIXRKRXX

SEQ ID NO: 7 - protein, 19 aa, Artificial Sequence
  Mpep
  MISC_FEATURE (1)..(1): X is an amino acid other than valine
  MISC_FEATURE (2)..(2): X is an amino acid other than leucine
  MISC_FEATURE (4)..(4): X is an amino acid other than threonine
  MISC_FEATURE (11)..(11): X is an amino acid other than serine
  MISC_FEATURE (14)..(14): X is an amino acid other than lysine
  MISC_FEATURE (18)..(18): X is an amino acid other than glutamine
  MISC_FEATURE (19)..(19): X is an amino acid other than glutamine
XXTXGLAALIXWIXRKRXX

SEQ ID NO: 8 - protein, 19 aa, Artificial Sequence
  Mpep
  MISC_FEATURE (1)..(1): X is an amino acid other than valine
  MISC_FEATURE (2)..(2): X is an amino acid other than leucine
  MISC_FEATURE (4)..(4): X is an amino acid other than threonine
  MISC_FEATURE (7)..(7): X is an amino acid other than proline
  MISC_FEATURE (14)..(14): X is an amino acid other than lysine
  MISC_FEATURE (18)..(18): X is an amino acid other than glutamine
  MISC_FEATURE (19)..(19): X is an amino acid other than glutamine
XXTXGLXALIAWIXRKRXX

SEQ ID NO: 9 - protein, 19 aa, Artificial Sequence
  Mpep
  MISC_FEATURE (1)..(1): X is an amino acid other than valine
  MISC_FEATURE (2)..(2): X is an amino acid other than leucine
  MISC_FEATURE (4)..(4): X is an amino acid other than threonine
  MISC_FEATURE (7)..(7): X is an amino acid other than proline
  MISC_FEATURE (11)..(11): X is an amino acid other than serine
  MISC_FEATURE (18)..(18): X is an amino acid other than glutamine
  MISC_FEATURE (19)..(19): X is an amino acid other than glutamine
XXTXGLXALIXWIARKRXX

SEQ ID NO: 10 - protein, 19 aa, Artificial Sequence
  Mpep
  MISC_FEATURE (1)..(1): X is an amino acid other than valine
  MISC_FEATURE (2)..(2): X is an amino acid other than leucine
  MISC_FEATURE (4)..(4): X is an amino acid other than threonine
  MISC_FEATURE (7)..(7): X is an amino acid other than proline
  MISC_FEATURE (11)..(11): X is an amino acid other than serine
  MISC_FEATURE (14)..(14): X is an amino acid other than lysine
  MISC_FEATURE (19)..(19): X is an amino acid other than glutamine
XXTXGLXALIXWIXRKRAX

SEQ ID NO: 11 - protein, 19 aa, Artificial Sequence
  Mpep
  MISC_FEATURE (1)..(1): X is an amino acid other than valine
  MISC_FEATURE (2)..(2): X is an amino acid other than leucine
  MISC_FEATURE (4)..(4): X is an amino acid other than threonine
  MISC_FEATURE (7)..(7): X is an amino acid other than proline
  MISC_FEATURE (11)..(11): X is an amino acid other than serine
  MISC_FEATURE (14)..(14): X is an amino acid other than lysine
  MISC_FEATURE (18)..(18): X is an amino acid other than glutamine
XXTXGLXALIXWIXRKRXA

SEQ ID NO: 12 - protein, 19 aa, Artificial Sequence
  Mpep
ALTTGLPALISWIKRKRQQ

SEQ ID NO: 13 - protein, 19 aa, Artificial Sequence
  Mpep
VATTGLPALISWIKRKRQQ

SEQ ID NO: 14 - protein, 19 aa, Artificial Sequence
  Mpep
VLATGLPALISWIKRKRQQ

SEQ ID NO: 15 - protein, 19 aa, Artificial Sequence
  Mpep
VLTAGLPALISWIKRKRQQ

SEQ ID NO: 16 - protein, 19 aa, Artificial Sequence
  Mpep
VLTTALPALISWIKRKRQQ

SEQ ID NO: 17 - protein, 19 aa, Artificial Sequence
  Mpep
VLTTGAPALISWIKRKRQQ

SEQ ID NO: 18 - protein, 19 aa, Artificial Sequence
  Mpep
VLTTGLAALISWIKRKRQQ

SEQ ID NO: 19 - protein, 19 aa, Artificial Sequence
  Mpep
VLTTGLPAAISWIKRKRQQ

SEQ ID NO: 20 - protein, 19 aa, Artificial Sequence
  Mpep
VLTTGLPALASWIKRKRQQ

SEQ ID NO: 21 - protein, 19 aa, Artificial Sequence
  Mpep
VLTTGLPALIAWIKRKRQQ

SEQ ID NO: 22 - protein, 19 aa, Artificial Sequence
  Mpep
VLTTGLPALISAIKRKRQQ

SEQ ID NO: 23 - protein, 19 aa, Artificial Sequence
  Mpep
VLTTGLPALISWAKRKRQQ

SEQ ID NO: 24 - protein, 19 aa, Artificial Sequence
  Mpep
VLTTGLPALISWIARKRQQ

SEQ ID NO: 25 - protein, 19 aa, Artificial Sequence
  Mpep
VLTTGLPALISWIKAKRQQ

SEQ ID NO: 26 - protein, 19 aa, Artificial Sequence
  Mpep
VLTTGLPALISWIKRARQQ

SEQ ID NO: 27 - protein, 19 aa, Artificial Sequence
  Mpep
VLTTGLPALISWIKRKAQQ

SEQ ID NO: 28 - protein, 19 aa, Artificial Sequence
  Mpep
VLTTGLPALISWIKRKRAQ

SEQ ID NO: 29 - protein, 19 aa, Artificial Sequence
  Mpep
VLTTGLPALISWIKRKRQA

SEQ ID NO: 30 - protein, 19 aa, Artificial Sequence
  Mpep
VLTTGLPALISWIKRKRQA

SEQ ID NO: 31 - protein, 19 aa, Artificial Sequence
  Mpep
VLAAGLPALISWIKRKRQQ

SEQ ID NO: 32 - protein, 19 aa, Artificial Sequence
  Mpep
VLATGLPALIAWIKRKRQQ

SEQ ID NO: 33 - protein, 19 aa, Artificial Sequence
  Mpep
VLTTGAAAAISWIKRKRQQ

SEQ ID NO: 34 - protein, 19 aa, Artificial Sequence
  Mpep
VLTTGLPALISWIKRKRAA

SEQ ID NO: 35 - protein, 19 aa, Artificial Sequence
  Mpep
VLATGLPALISWIKAAAQA

SEQ ID NO: 36 - protein, 5 aa, Artificial Sequence
  Linker
GGGGS

SEQ ID NO: 37 - protein, 5 aa, Artificial Sequence
  Linker
EAAAK

SEQ ID NO: 38 - protein, 20 aa, Artificial Sequence
  Linker
LEAGCKNFFPRSFTSCGSLE

SEQ ID NO: 39 - protein, 12 aa, Artificial Sequence
  Linker
CRRRRRREAEAC

SEQ ID NO: 40 - protein, 5 aa, Artificial Sequence
  Linker
RLRWR

SEQ ID NO: 41 - protein, 17 aa, Artificial Sequence
  Linker
GRPRESGKKRKRKRLKP

SEQ ID NO: 42 - protein, 13 aa, Artificial Sequence
  Linker
GRKKRRQRRRPPQ

SEQ ID NO: 43 - protein, 5 aa, Artificial Sequence
  Linker
RYIRS

SEQ ID NO: 44 - protein, 7 aa, Artificial Sequence
  Linker
RRMKWKK

SEQ ID NO: 45 - protein, 9 aa, Artificial Sequence
  Linker
  MISC_FEATURE (9)..(9): X is 1-4 arginines
RRRRRRRRX

SEQ ID NO: 46 - protein, 12 aa, Artificial Sequence
  Linker
RRRRRRRRRFFC

SEQ ID NO: 47 - protein, 14 aa, Artificial Sequence
  Linker
KLAKLAKKLAKLAK

SEQ ID NO: 48 - protein, 26 aa, Artificial Sequence
  Scrambled peptide
LSRQRALQIKGLIKTPWKIAGVLGVT

SEQ ID NO: 49 - protein, 14 aa, Artificial Sequence
  TAMpep114
GIGAVLKVLTTGLP

SEQ ID NO: 50 - protein, 20 aa, Artificial Sequence
  TAMpep120
GIGAVLKVLTTGLPALISWI

SEQ ID NO: 51 - protein, 13 aa, Artificial Sequence
  TAMpep820
VLTTGLPALISWI

SEQ ID NO: 52 - protein, 15 aa, Artificial Sequence
  TAMpep822
VLTTGLPALISWIKR

SEQ ID NO: 53 - protein, 17 aa, Artificial Sequence
  TAMpep1026
TTGLPALISWIKRKRQQ

SEQ ID NO: 54 - protein, 15 aa, Artificial Sequence
  TAMpep1226
GLPALISWIKRKRQQ

SEQ ID NO: 55 - protein, 12 aa, Artificial Sequence
  TAMpep1526
ALISWIKRKRQQ